(19) United States
(12) Patent Application Publication
(10) Pub. No.: US 2006/0014140 A1
(43) Pub. Date: Jan. 19, 2006

(54) Molecular methods and compositions for detecting and quantifying respiratory viruses

(76) Inventor: Guy Boivin, Sainte-Foy (CA)

Correspondence Address: OGILVY RENAULT LLP, 1981 MCGILL COLLEGE AVENUE, SUITE 1600, MONTREAL, QC, H3A2Y3, CA

(21) Appl. No.: 11/155,478

(22) Filed: Jun. 20, 2005

Related U.S. Application Data
(63) Continuation-in-part of application No. PCT/CA03/01994, filed on Dec. 19, 2003.

(30) Foreign Application Priority Data
Dec. 19, 2002 (CA) 2,411,264
Jan. 24, 2003 (CA) 2,418,004

(51) Int. Cl. C12Q 1/70; C12Q 1/68

(52) U.S. Cl. 435/5; 435/6

(57) ABSTRACT

The present invention relates to new methods and nucleic acid sequences for the detection and quantification of human metapneumovirus.

### CROSS-REFERENCE TO RELATED APPLICATIONS

This application is a Continuation-In-Part application of PCT/CA03/01994 filed Dec. 19, 2003, which claims the benefit of Canadian Patent Application Serial No. 2,411,264 filed Dec. 19, 2002, and of Canadian Patent Application Serial No. 2,418,004, filed Jan. 24, 2003, the entire disclosure of which are incorporated by reference.

### BACKGROUND OF THE INVENTION

1. Field of the Invention

This application relates to the field of microrganisms detection and more particularly to the detection of respiratory tract viruses using virus specific nucleic acids.

2. Related Art

Respiratory tract infections are a significant cause of morbidity and mortality in all age groups but especially in young children, elderly subjects, and immunocompromised patients. Most of these infections are caused by viruses with influenza A and B and the human respiratory syncytial virus (hRSV) being associated with the most frequent and severe complications i.e. pneumonitis and death (Dowell et al., 1996, J. Infect. Dis. 174:456-462; Simonsen et al., 2000, J. Infect. Dis. 181:831-837; Han et al., 1999, J. Infect. Dis. 179:25-30; Neuzil et al., 2000, N. Engl. J. Med. 342:225-231; Simoes, 1999, Lancet 354:847-852.). Other viruses such as adenoviruses (>50 serotypes), parainfluenza viruses (PIV) (4 serotypes), rhinoviruses (>100 serotypes); and coronaviruses also contribute to acute respiratory tract infections (Treanor, 2002, Clinical Virology, 2^{nd} ed., ASM Press, Washington, D.C., 7-26). Also, recent studies by the inventor and others have showed that the new human metapneumovirus (hMPV) should be added to the list of human respiratory viral pathogens in all age groups (van den Hoogen et al., 2001, Nat. Med. 7:719-724; Peret et al., 2002, J. Infect. Dis. 185:1660-1663; Boivin et al., 2002, J. Infect. Dis. 186:1330-1334).

The hMPV was initially isolated from nasopharyngeal aspirates recovered from 28 children suffering from respiratory tract infections. Based on partial nucleic acid sequences, gene organization, and electron microscopy (EM) findings, this virus was preliminary assigned to the Paramyxoviridae family, subfamily Pneumovirinae, genus Metapneumovirus. Since then, hMPV has been isolated in North America by our group (Peret et al., 2002, J. Infect. Dis. 185:1660-1663), in Australia (Nissen et al., 2002, Med. J. Aust. 176:188), and in England (Stockton et al., 2002, Emerg. Infect. Dis. 8:897-901).

Preliminary Virological Characteristics of hMPV.

The initial report by Van Den Hoogen et al. (van den Hoogen et al., 2001, Nat. Med. 7:719-724) indicated that the new virus replicated slowly in tertiary monkey kidney (TMK) cells with cytopathic effects (CPE) consisting of syncytium formation appearing 10-14 days post inoculation. Notably, the virus grew very poorly in Vero and A549 cells and could not be propagated in Madin Darby canine kidney (MDCK) cells. Virus-infected TMK cell-culture supernatants also did not display any hemagglutinating activity. The supernatants of infected TMK cells were used for EM studies, which revealed the presence of paramyxovirus-like pleomorphic particles in the range of 150-600 nm with short envelope projections in the range of 13-17 nm. However, RT-PCR studies with primer sets specific for known paramyxoviruses were initially all negative. Using a random PCR amplification procedure known as RAP-PCR (Welsh et al., 1990, Nucleic Acids Res. 18:7213-7218; Ralph et al., 1993, Proc. Natl. Acad. Sci. U.S.A. 90:10710-10714), the Dutch investigators identified differentially displayed bands (compared to human parainfluenza-1 virus) which, upon sequencing, were found to be most closely related to the avian pneumovirus (APV).

APV, the agent of turkey rhinotracheitis, has been the sole member of the recently assigned Metapneumovirus genus within the Pneumovirinae subfamily (Taxonomy Virus, 2000, Seventh Report of the International Committee on Taxonomy of Viruses. San Diego. 551, 557, 559-560). The latter virus is a major respiratory pathogen of turkeys and other domestic fowl worldwide (Yu et al., 1992, Virology. 186:426-434; Jones et al., 1987, Vet. Rec. 120:301-302; Jones, 1996, Avian Pathol. 25:639-648; Seal et al., 2000, Virus Res. 66:139-147). There are four serotypes of APV with serotype C (present in US) being the most closely related to hMPV (van den Hoogen et al., 2001, Nat. Med. 7:719-724). The metapneumoviruses (including APV and hMPV) are structurally similar to the human respiratory syncytial virus (hRSV), which also belongs to the Pneumovirinae subfamily, except for two features: they lack the two non-structural proteins (NS1 and NS2) and they have a different genomic organization (Yu et al., 1992, Virology. 186:426-434). Recently, an almost complete hMPV sequence (13.4 kb) was reported in GenBank (accession number: AF371337) (van den Hoogen et al., 2002, Virology. 295:119-132). The overall percentage of amino acid sequence identity for the N (nucleoprotein), P (phosphoprotein), M (matrix), F (fusion), M2 (membrane), and L (polymerase) proteins is in the range of 56-88% between hMPV and APV whereas it is 18-44% between hMPV and hRSV. However, the G (glycoprotein) and SH (small hydrophobic) proteins of hMPV do not possess significant amino acid sequence identity with those of any other viruses. In contrast to APV, hMPV was shown to be unable to replicate in turkeys and chickens but the human virus replicated efficiently in the respiratory tract of cynomolgous macaques inducing upper respiratory tract symptoms that correlated with peak viral replication in throat samples between 2 to 8 days post infection (van den Hoogen et al., 2001, Nat. Med. 7:719-724). Thus, these preliminary data indicated that hMPV was a primate pathogen associated with respiratory diseases.

Clinical Importance of hMPV.

There is little information about the clinical presentation of hMPV infection. In the original report by Van Den Hoogen et al., 27 of the 28 infected children from the Netherlands were below the age of five (van den Hoogen et al., 2001, Nat. Med. 7:719-724). It was mentioned that those infected patients were diagnosed with a range of acute respiratory tract infections (ARTI) from mild to severe including bronchiolitis and pneumonia although no detailed clinical data were provided. In a limited study conducted in the winter of 2000, the same authors found that 7 (≈10%) of 68 pediatric patients with unexplained ARTI had evidence of acute hMPV infection by RT-PCR (van den Hoogen et al., 2001, Nat. Med. 7:719-724). In addition, Australian investigators reported the detection of hMPV by RT-PCR in 3/200 (1.5%) nasopharyngeal aspirates from children whose specimens were negative for the common respiratory viruses during the winter of 2001 (Nissen et al., 2002, Med. J. Aust. 176:188). Recently, hMPV RNA was detected by RT-PCR in 9/405 (2.2%) of nasopharyngeal swabs from English patients who consulted for influenza-like illnesses and had negative tests for other pathogens (Stockton et al., 2002, Emerg. Infect. Dis. 8:897-901). These studies, however, suffer from major limitations such as their small size (in most cases), the superficial description of clinical features, the lack of morbidity/mortality data, their retrospective nature, the exclusion of patients who tested positive for other viruses, and the absence of a control group.

Thus far, seroprevalence studies have been limited to the Dutch population. An indirect immunofluorescence assay (IFA) using infected TMK cells has revealed that 25% of the children aged between 6 and 12 months had hMPV antibodies and 70% had become seropositive by the age of 5 years (n=60) (van den Hoogen et al., 2001, Nat. Med. 7:719-724). Retrospective serological testing also indicates that hMPV has been circulating in the Dutch population for at least half a century.

Diagnosis of hMPV Infections

hMPV has been recently reported in America (Peret et al., 2002, J. Infect. Dis. 185:1660-1663). 11 hMPV strains were isolated on LLC-MK2 cells, a continuous cell line from rhesus monkey kidneys that is used in our center to recover parainfluenza viruses (PIV). Electron microscopy studies of these isolates revealed pleomorphic, spherical and filamentous particles with characteristics (nucleocapsid diameter and pitch spacing, length of projections) that were consistent with paramyxoviruses. RT-PCR and immunofluorescent assays for paramyxoviruses (PIV 1-3, hRSV) and other respiratory viruses (adenoviruses, coronaviruses, influenza viruses A and B, rhinoviruses) were negative for all 11 isolates. In contrast, amplification of the hMPV F (fusion) and M (matrix) genes was positive for all isolates (Peret et al., 2002, J. Infect. Dis. 185:1660-1663).

Subsequently, the virological features and clinical findings associated with a larger set of 38 hMPV strains from subjects hospitalized (at CHUL, Québec City) since 1993 were examined (Boivin et al., 2002, J. Infect. Dis. 186:1330-1334). With all cell lines used, albeit was possible to determine that the previously unidentified hMPV strains only consistently grew on LLC-MK2 cells after a mean incubation time of 17.3 days (range 3-23 days). Interestingly, most isolates recovered before 2002 produced only limited syncytia on LLC-MK2 cells, whereas some of the recent strains produced large syncytia typical of those induced by hRSV. A seroconversion for hMPV wa observed in two adult patients for whom acute and convalescent sera were available using an IFA test designed in our laboratory. Such seroconversion episodes (titers from < 1/10 to 1/80 and from < 1/10 to 1/160 over 2 and 4 weeks, respectively) occurred during the course of the patients' illness showing that hMPV was a true respiratory pathogen. A phylogenetic analysis of hMPV Canadian strains based on F gene sequences identified two major phylogenetic groups. At the nucleotide level, we found a similarity of 94.2-100% and 92.6-100% for isolates of groups 1 and 2, respectively, whereas the similarity at the amino acid level was higher i.e. 96.8-100% (group 1) and 97.2-100% (group 2) showing a functional/immunological conservation of the F protein. Interestingly, co-circulation of the two hMPV groups during some years was observed.

Over the last decade, most (86.7%) hMPV strains from Quebec area were isolated during a 5-month period, from December to May. As an indication of the relative frequency of detection of hMPV compared to other respiratory viruses, all respiratory samples were retrieved (n=862) and send to virology laboratory for viral culture during the winter season (December to May) of 2000-01. During this period, hMPV was recovered in 2.3% of all respiratory samples tested by viral culture compared to 2.3% for PIV, 3.4% for adenoviruses, 4.8% for hRSV, and 19.8% for influenza A or B viruses. Overall, hMPV represented 7.1% of all positive cultures from respiratory samples during that winter season with percentages of 4.1%, 2.8%, and 29.3% for patients aged 0-5, 5-65, and >65 years, respectively. A retrospective review of the clinical charts from hMPV-infected subjects identified for the first time the role of this virus as a cause of severe lower respiratory tract infections in all age groups but especially in young children, elderly subjects, and immunocompromised patients. More specifically, the most frequent diagnoses associated with hMPV infection in hospitalized children were pneumonitis (66%) and bronchiolitis (58.3%) whereas bronchitis/bronchospasm (60%) and pneumonitis (40%) were most commonly seen in elderly subjects (Boivin et al., 2002, J. Infect. Dis. 186:1330-1334). Although no histopathological studies were available, hMPV was the only pathogen identified in respiratory tract secretions of 3 subjects (including 2 with immunosuppressive conditions) who died of respiratory insufficiency.

Respiratory viruses have been classically identified by viral culture using a variety of permissive cell lines. However, viral culture is not convenient for clinical management due to the need of specialized techniques (cell culture, immunofluorescence methods), the long turnaround time before appearance of typical cytopathic effects for many viruses (up to a few weeks), and the need for rapid inoculation of an infectious virus into multiple cell lines for optimal sensitivity (Specter et al., 2002, Clinical Virology, 2^{nd} ed., ASM Press, Washington, D.C., 0.243-272).

Antigenic detection kits for some respiratory viruses (influenza viruses and hRSV) have been developed by many companies. Most are immunoenzymatic assays (ELISA) based on the capture of a viral antigen by a specific monoclonal antibody. Although such assays eliminate many of the problems inherent to viral cultures (no need for specialized equipment, rapid turnaround time in <30 min, no need to recover an infectious virus), they have not been widely adopted due to poor sensitivity and/or specificity compared to viral culture. For instance, the sensitivity and specificity of some rapid antigenic tests for influenza has varied from 46 to 96% and from 52 to 99%, respectively, compared to viral culture. The performance of these tests is dependent on many variables such as the patient's age, the type of specimen and the duration of the disease (Hayden et al., 2002, Clinical Virology, 2^{nd} ed., ASM Press, Washington, D.C., 891-920). The performance of the rapid antigenic tests for hRSV has been considered quite good in children with sensitivity and specificity varying from 80 to 95% (Piedra et al., 2002, Clinical Virology, 2^{nd} ed., ASM Press, Washington, D.C., 763-790) but poor in adults (Englund et al., 1996, J. Clin. Microbiol. 34:1649-1653). Detection of antibodies against viral pathogens (serology) has a limited value in the diagnosis of acute viral infections since two serums, collected at a 2 to 3 week interval, are required in order to document a significant rise in viral antibody titers.

More recently, PCR or RT-PCR assays have been developed for many respiratory viruses allowing detection of small amounts of viral DNA or RNA in a clinical sample. In the so-called “multiplex” format, PCR assays have been designed to amplify more than one viral target in the same PCR reaction (Ellis et al., 1997, J. Clin. Microbiol. 35:2076-2082; Fan et al., 1998, Clin. Infect. Dis. 26:1397-1402; Liolios et al., 2001, J. Clin. Microbiol. 39:2779-2783; Grondahl et al., 1999, J. Clin. Microbiol. 37:1-7). However, most multiplex PCR assays reported to date do not allow the simultaneous detection of all viruses involved in acute respiratory tract infections of humans and they require separate steps for the amplification and detection of viral genes, greatly increasing the assay's turnaround time and preventing reliable quantification of the amplicons. At present, only one multiplex PCR assay (the Hexaplex test from Prodesse, Waukesha, Wis.) is commercially-available. The Hexaplex detects 6 respiratory viruses (influenza A and B, PIV 1-3 and hRSV) using specific viral probes in a microplate format (Fan et al., 1998, Clin. Infect. Dis. 26:1397-1402; Kehl et al., 2001, J. Clin. Microbiol. 39:1696-1701). This assay does not use real-time amplification methodologies and has a turnaround time of about one full working day. Thus, there is a need for developing more rapid molecular assays that permit detection of a larger panel of respiratory viral pathogens.

Rapid and sensitive detection of respiratory viruses is an important clinical goal to help physicians select the most appropriate antiviral, reduce nosocomial transmission of pathogens, decrease the length of hospitalization, and reduce antibiotic use and bacterial resistance. In view of the above, it will be appreciated that diagnosis assay that can rapidly detect the presence of many respiratory viruses is needed.

### SUMMARY OF THE INVENTION

One object of the present invention to provide a rapid and sensitive method using amplification primers and/or probes for determining the presence and/or the amount in a test sample of nucleic acids from clinically important respiratory viral pathogens including human metapneumovirus.

Another aspect there is provided multiplex assays aimed at detecting conserved genes of common respiratory viral pathogens such as influenza A and B, human respiratory syncytial virus (hRSV) A and B, human metapneumovirus (hMPV), parainfluenza viruses (PIV) types 1-4, adenoviruses, rhinoviruses, enteroviruses, and coronaviruses.

The test sample is usually of clinical or environmental sources.

The nucleic acid amplification method is preferably but not exclusively PCR. In a preferred embodiment, identification of the amplicons produced by the amplification of nucleic acids from respiratory viruses is achieved by verifying the specific melting temperature of amplified viral products or by hybridization with specific viral probes. Other amplification technologies including target and probe amplification techniques as well as signal amplification techniques performed in liquid phase or onto solid supports may also be used.

One object of the present invention is to provide a method using nucleic acid amplification and/or detection for determining the presence and/or the amount of nucleic acids of the human metapneumovirus (hMPV). The nucleic acid amplification method is PCR. However, other amplification technologies including target and probe amplification techniques as well as signal amplification performed in liquid phase or onto solid support may also be used.

The present invention also provides a method to detect the presence of hMPV in a test sample based on the detection of a nucleotide sequence from either the nucleocapsid (N), phosphoprotein (P), matrix (M), fusion (F), membrane (M2), polymerase (L), glycoprotein (G), and small hydrophobic (SH) genes. In a particularly preferred embodiment, the method to detect the presence of hMPV in a test sample is based on the detection of a nucleotide sequence from the nucleoprotein (N) or polymerase (L) genes. Identification of amplified hMPV products can be achieved by verifying the specific melting temperature of amplified viral products or by hybridization with hMPV-specific probes.

According to a particular object of the present invention, all sequences can be choosen from two new sequences of strains CAN9783 and CAN9875 of hMPV, registered at the International Depository Authority of Canada, and comprising the nucleic acid sequences SEQ ID NOs 173 and 174.

According to another object, there is provided a method for typing hMPV strains which comprises the steps of reproducing the above amplification method followed by either hybridization using type-specific probes, DNA sequencing or restriction enzyme analysis. Typing is done preferentially, but not exclusively, on the most variable hMPV genes i.e. the fusion (F) and glycoprotein (G) genes.

All DNA fragments and oligonucleolides useful in the detection or identification of a hMPV strain are further objects of this invention as well as any composition of matters that include them (recombinant vectors, hosts, diagnostic kits).

### BRIEF DESCRIPTION OF THE DRAWINGS

Further features and advantages of the present invention will become apparent from the following detailed description, taken in combination with the appended drawings, in which:

FIG. 1 illustratess a melting curve analysis of amplified hMPV strains using specific nucleoprotein primers (Sequence ID Nos. 92 and 94);

FIG. 2 shows an ethidium bromide-stained agarose gel showing restriction fragment length polymorphism of amplified hMPV fusion (F) gene sequences digested with enzymes ApaL I and Avr II.PCR products of the hMPV F gene (amplified with primers sequence ID Nos. 97 and 98) were digested with ApaL I and Avr II. Strains belonging to hMPV genotype F-I were digested only by ApaL I resulting in two fragments of 532 bp and 227 bp whereas strains belonging to hMPV genotype F-2 were digested only by Avr II resulting in fragments of 447 and 312 bp.;

FIGS. 3A to 3D show the age at admission of children hospitalized for acute respiratory tract infections (ARTI) caused by hMPV (A), hRSV (B), and influenza A (C) as well as for the whole study population (D) described in example 4;

FIG. 4 represents the biweekly distribution of virologically cases with acute respiratory tract infections and their controls in the study reported in example 4;

FIG. 5 represents the biweekly distribution of HMPV isolates in the study group (hospitalized children) described in example 4 and in respiratory specimens tested at the regional virology laboratory (RVL);

FIG. 6 illustrates a phylogenetic tree of hMPV strains recovered as part of the study described in example 4 also including the prototype strain from the Netherlands (GenBank af371367) and a Canadian isolate from season 2000-01 (hMPV35-2001). The dates of sample collection are in parentheses;

FIGS. 7A and 7B illustrate a melting curve analysis of viral strains amplified using the respiratory-1 multiplex real-time PCR assay. A—Multiplex real-time PCR assay using hMPV N primers (SEQ ID NOS. 95 and 96). B—Multiplex real-time PCR assay using hMPV L primers (SEQ ID NOS. 112 and 114). Note: the internal control is apparent (Tm: 91° C.) in the absence of amplification of specific viral products;

FIG. 8 illustrates a melting curve analysis of viral strains amplified using the respiratory-2 multiplex real-time PCR assay. Note: The internal control is apparent (Tm: 91° C.) in the absence of amplification of specific viral products;

FIGS. 9A and 9B illustrate a fluorescence signal as a function of elongation step for the determination of the hMPV load in clinical samples using quantitative real-time PCR (A); and a standard curve for determination of the hMPV using quantitative real-time PCR (B);

FIG. 10A illustrates a phylogenetic tree for the F gene of hMPV;

FIG. 10B illustrates a phylogenetic tree for the G gene of hMPV;

FIG. 11 illustrates a melting curve analysis of amplified viral genes and of an internal control as determined by the LightCycler™ instrument. Note that high amounts of amplified viral products may preclude detection of the internal control in some PCR runs; and

FIG. 12 illustrates a melting curve analysis SARS-coronavirus using a real time PCR assay.

### DETAILED DESCRIPTION OF THE PREFERRED EMBODIMENT

The invention provides nucleic acid based diagnostic methods and compositions to detect and quantify respiratory viruses.

In a preferred embodiment respiratory viral pathogens targeted by the diagnostic assays include all genotypes of influenza A and B viruses, hRSV, hMPV, PIV, adenoviruses, rhinoviruses, and enteroviruses. Other less common (coronaviruses, reoviruses) and yet to be discovered respiratory viruses are also targets under the scope of the present invention.

In the description of this invention, the terms [symbol] nucleic acids [symbol] and [symbol] sequences [symbol] might be used interchangeably. However, [symbol] nucleic acids [symbol] are chemical entities while [symbol] sequences [symbol] are the pieces of information encoded by these [symbol] nucleic acids [symbol] . Both nucleic acids and sequences are equivalently valuable sources of information for the matter pertaining to this invention.

In one embodiment the method targets conserved hMPV nucleotide sequences for diagnostic purposes and variable hMPV sequences for epidemiological typing purposes. Nucleotide sequences were selected either from a number of hMPV viral sequences obtained in the inventor's laboratory or from public databases (GenBank accession number AF371337).

Our proprietary DNA fragments and oligonucleotides (a non-restrictive list of primers and probes) are listed in the following tables.

The detection of the viruses including the hMPV is conducted through compositions of matters such as diagnostic kits comprising the amplification primers or probes of this invention.

In the methods and kits of the present invention, probes and primers are not limited to nucleic acids and may include, but are not restricted to, analogs of nucleotides. The diagnostic reagents constituted by the probes and the primers may be present in any suitable form (bound to a solid support, liquid, lyophilized, etc.) for detection and/or amplification of nucleic acid sequences of the viruses.

In the methods and kits of the present invention, amplification reactions may include but are not restricted to: a) polymerase chain reaction (PCR), b) ligase chain reaction (LCR), c) nucleic acid sequence-based amplification (NASBA), d) self-sustained sequence replication (3SR), e) strand displacement amplification (SDA), f) branched DNA signal amplification (bDNA), g) transcription-mediated amplification (TMA), h) cycling probe technology (CPT), i) nested PCR, j) multiplex PCR, k) solid phase amplification (SPA), l) nuclease dependent signal amplification (NDSA), m) rolling circle amplification technology (RCA), n) anchored strand displacement amplification, o) solid-phase (immobilized) rolling circle amplification.

In the methods and kits of the present invention, detection of the nucleic acids of target genes may include real-time or post-amplification technologies. These detection technologies can include, but are not limited to, the use of intercalating agents such as SYBR green, fluorescence resonance energy transfer (FRET)-based methods such as adjacent hybridization of probes (including probe-probe and probe-primer methods), TaqMan probe, molecular beacon probe, Scorpion probe, nanoparticle probe and Amplifluor probe. Other detection methods include target gene nucleic acids detection via immunological methods, solid phase hybridization methods on filters, chips or any other solid support. In these systems, the hybridization can be monitored by fluorescence, chemiluminescence, potentiometry, mass spectrometry, plasmon resonance, polarimetry, colorimetry, flow cytometry or scanometry. Nucleotide sequencing, including sequencing by dideoxy termination or sequencing by hybridization (e.g. sequencing using a DNA chip) represents another method to detect and characterize the nucleic acids of target genes.

Amplification of hMPV Sequences

In one aspect of the invention there is provided methods and compositions for detecting hMPV in a sample.

In one embodiment, a reverse-transcription (RT)-PCR protocol is used for nucleic acid amplification. The single-stranded viral RNA is converted to complementary DNA using an enzyme possessing a reverse transcriptase activity and a specific hMPV primer. Alternatively, random hexamer primers can be substituted to the specific hMPV primer. In a preferred embodiment, viral RNA was prealably extracted from 200 μl of naso-pharyngeal aspirates using the QIAamp Viral RNA Mini Kit (QIAGEN). Complementary DNA was synthesized using 10 μl of eluted RNA, 0.75 μM of a specific hMPV primer (Table 2, sequences ID Nos. 91, 97, 100, 102, 109, 112), and the Omniscript Reverse Transcriptase Kit (QIAGEN, Mississauga, ON, Canada) following the manufacturer's recommendations.

For DNA amplification by the widely used PCR method, primer pairs were derived from our proprietary DNA fragments or from public database sequences (Table 1). During DNA amplification by PCR, two oligonucleotide primers binding respectively to each strand of the heat-denatured target DNA from the microbial genome are used to amplify exponentially in vitro the target DNA by successive thermal cycles allowing denaturation of the DNA, annealing of the primers and synthesis of new targets at each cycle (Persing, 1993, Diagnostic Molecular Microbiology: Principles and Applications, American Society for Microbiology, Washington, D.C. 88-109).

In another embodiment, complementary DNA was amplified using a real-time PCR procedure with the LC Faststart DNA Master SYBR Green 1 Kit (Roche Diagnostics, Laval, QC, Canada) or the SYBR Green TAQ ReadyMix for quantitative PCR capillary formulation (Sigma-Aldrich, Oakville, ON) in a LightCycler instrument (Roche Diagnostics). Each reaction had a total volume of 20 μl including 2 μl of RT mixture and 18 μl of a reaction mixture containing 4 mM MgCl₂, 2 μl of Faststart DNA SYBR Green Master Mix or the SYBR Green TAQ ReadyMix for quantitative PCR capillary formulation, 3% DMSO, and 0.5 μM of each hMPV primer (see Tables 2 and 4 for sequence ID Nos. and specific combinations of primers for amplification, respectively). Cycling conditions typically included an initial denaturation step of 10 min. at 94° C., followed by 50 cycles of 15 s at 94° C., 5 s at 54° C., and 30 s at 72° C. However, the cycling conditions could slightly vary according to the hMPV gene to amplify.

The person skilled in the art of nucleic acid and/or signal amplification knows the existence of other rapid amplification procedures such as ligase chain reaction (LCR), reverse transcriptase PCR (RT-PCR), transcription-mediated amplification (TMA), self-sustained sequence replication (3SR), nucleic acid sequence-based amplification (NASBA), strand displacement amplification (SDA), branched DNA (bDNA), cycling probe technology (CPT), solid phase amplification (SPA), rolling circle amplification technology (RCA), solid phase RCA, anchored SDA and nuclease dependent signal amplification (NDSA) (Persing, 1993, Diagnostic Molecular Microbiology: Principles and Applications, American Society for Microbiology, Washington, D.C. 88-109; Lee, 1997, Nucleic Acid Amplification Technologies: Applications to Disease Diagnosis, Eaton Publishing, Boston, Mass. 61-125; Westin et al., 2000, Nat. Biotechnol. 18:199-204). The scope of this invention is not limited to the use of amplification by PCR, but rather includes the use of any nucleic acid amplification method or any other procedure which may be used to increase the sensitivity and/or the rapidity of nucleic acid-based diagnostic tests. The scope of the present invention also covers the use of any nucleic acid and/or signal amplification and detection technology including real-time or post-amplification detection technologies, any amplification technology combined with detection, any hybridization nucleic acid chips or array technologies, any amplification chips or combination of amplification and hybridization chip technologies. Detection and identification by any nucleotide sequencing method is also under the scope of the present invention.

Any oligonucleotide, useful for diagnosis, and which are derived from hMPV sequences and used with any nucleic acid amplification and/or hybridization technologies are also under the scope of this invention.

Detection of Amplification Products

Classically, the detection of PCR amplification products is performed by standard ethidium bromide-stained agarose gel electrophoresis. Using this approach, primers specific for a nucleotide sequence from either the nucleocapsid (N), phosphoprotein (P), matrix (M), fusion (F), polymerase (L), glycoprotein (G), genes generated amplicons of various lengths depending on the selected primers pairs (Table 4). It is however clear that other detection methods of amplified products may be faster, more sensitive, and more practical. Amplicon detection may also be performed by solid support or liquid hybridization using strain-specific internal DNA probes hybridizing to an amplification product. Such probes may be generated from sequences from our repertory (Table 1) and designed to specifically hybridize to DNA amplification products which are objects of the present invention. Amplicons can also be characterized by DNA sequencing. Alternatively, a rapid detection method developed with real-time PCR assays consists of determining the specific melting temperature of amplicons at the end of the amplification reaction.

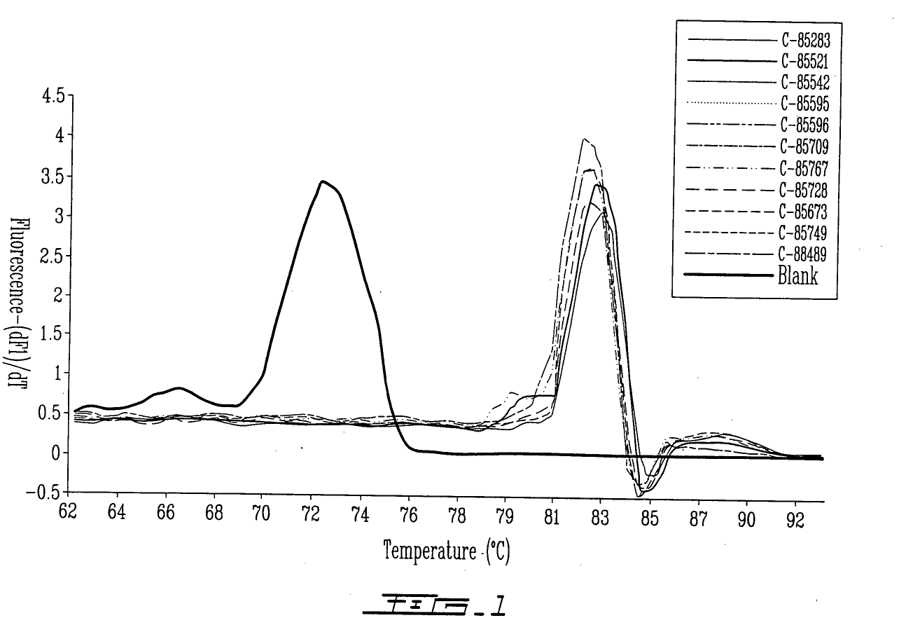

In one embodiment, detection of hMPV amplicons was characterized using the melting curve analysis feature of a real-time PCR instrument. Briefly, following the last amplification cycle on a LightCycler instrument, the internal temperature was rapidly increased to 94° C. then decreased to 60° C. for 30 seconds, followed by slowly increasing to 94° C. at a rate of 0.1° C. per second, with continuous fluorescence lecture. Using this procedure, the specific melting temperature obtained with the specific amplicons produced by the pairs of primers specific for the N (SEQ ID No 92+94), M (SEQ ID No 100+101), F (SEQ ID 97+98), P (SEQ ID No 102+103) and L (SEQ ID No 112+113) gene sequences was found to be 82.63° C.±0.87° C., 82.75° C.±0.80° C., 83.29° C.±0.45° C., 80.48° C.±0.46° C., and 77.99±0.33° C., respectively (Table 5 and see also FIG. 1 for example of the melting temperature obtained for different hMPV strains with the N primers). Alternatively, we also designed specific adjacent and TaqMan fluorescent probes for the various hMPV gene products (N, M, F, G, P and L) that can be used for real-time detection and quantification of amplicons (Table 3). Here, quantification of hMPV gene products is achieved through fluorescence resonance energy transfer (FRET) when the two probes hybridize nearby on a complementary DNA sequence. Other types of detection methods including the use of TaqMan or molecular beacon probes specific for the hMPV sequences described above are also under the scope of this invention.

Typing of hMPV

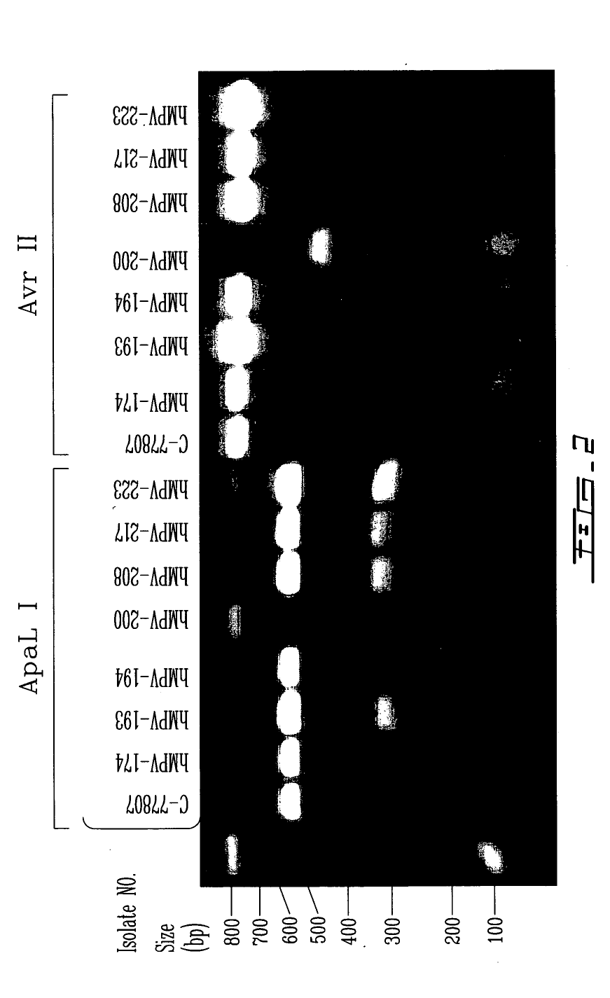

A rapid molecular typing method is provided herein for epidemiologic studies based on amplification of a portion of the F (fusion) glycoprotein (primer sequence ID Nos. 97 and 98, Tables 2 and 4) followed by DNA sequencing or digestion with restriction enzymes (ApaL I and Avr II). Table 6 summarizes the size of the F fragments obtained for different hMPV isolates following enzymatic digestion and the corresponding genotype (see also FIG. 2 for an example of the band pattern following electrophoresis on an agarose gel). Alternative typing methods based on the use of type-specific hMPV F primers and/or probes as well as DNA sequencing of amplified products are also within the scope of this invention. hMPV subtyping using similar methodologies for the other surface glycoprotein (i.e. the G gene) may also be used.

A method for detection of a plurality of potential hMPV strains having different genotypes may be conducted in separate reactions and physical enclosures, one type at the time. Alternatively, it could be conducted simultaneously for different types in separate physical enclosures, or in the same physical enclosures. In the latter scenario a multiplex PCR reaction could be conducted which would require that the oligonucleotides are all capable of annealing with a target region under common conditions. Since many probes or primers are specific for a determined genotype, typing an hMPV strain is under the scope of the present invention. When a mixture of oligonucleotides annealing together with more than one type of hMPV is used in a single physical enclosure or container, different detection labels would be used to distinguish one type from another.

The diagnostic kits, primers and/or probes mentioned above can be used to detect and/or identify hMPV, whether said diagnostic kits, primers and probes are used for in vitro or in situ applications. The said samples may include but are not limited to: any clinical sample, any environmental sample, any viral culture, any tissue, or any cell line.

According to another embodiment of the present invention the diagnostic kits, primers and/or probes can be used alone or in combination with any other assay suitable to detect and/or identify other microorganisms (i.e. viruses, bacteria, fungi, and parasites) including but not limited to: any assay based on nucleic acids detection, any immunoassay, any enzymatic assay, any biochemical assay, any lysotypic assay, any serological assay, any culture on specific cell lines, and any infectivity assay on animals.

In the methods and kits described herein below, the oligonucleotide probes and amplification primers have been derived from larger sequences (i.e. DNA fragments of at least 100 base pairs). All DNA sequences have been obtained either from our proprietary sequences (Table 1) or from SEQ ID No. 1 from public databases.

It will appear to the person skilled in the art that oligonucleotide sequences other than those described in the present invention and which are appropriate for detection and/or identification of hMPV may also be derived from sequences SEQ ID NOs 173 and 174. For example, the oligonucleotide primers or probes may be shorter but of a length of at least 10 nucleotides or longer than the ones chosen; they may also be selected anywhere else in the proprietary DNA fragments or in the sequences selected from public databases; they may also be variants of the same oligonucleotide. If the target DNA or a variant thereof hybridizes to a given oligonucleotide, or if the target DNA or a variant thereof can be amplified by a given oligonucleotide PCR primer pair, the converse is also true: a given target DNA may hybridize to a variant oligonucleotide probe or be amplified by a variant oligonucleotide PCR primer. Alternatively, the oligonucleotides may be designed from said DNA fragment sequences for use in amplification methods other than PCR. Consequently, the core of this invention is the detection and/or identification of hMPV by targeting DNA sequences which are used as a source of specific and ubiquitous oligonucleotide probes and/or amplification primers. Although the selection and evaluation of oligonucleotides suitable for diagnostic purposes require much effort, it is quite possible for the individual skilled in the art to derive, from the selected DNA fragments, oligonucleotides other than the ones listed in Tables 2 and 3 which are suitable for diagnostic purposes. When a proprietary fragment or a public database sequence is selected for its specificity and ubiquity (i.e. ability to detect all hMPV strains), it increases the probability that subsets thereof will also be specific and ubiquitous.

The DNA fragments have been obtained as a repertory of sequences created by amplifying hMPV nucleic acids with proprietary primers. These primers and the repertory of nucleic acids as well as the repertory of nucleotide sequences are further objects of this invention (Tables 1-3).

As part of the design rules, all oligonucleotides were evaluated for their efficiency for hybridization or PCR amplification by computer analysis using standard programs (i.e. the GCG Wisconsin package programs, the primer analysis software Oligo™ 6 and MFOLD 3.0). The potential suitability of the PCR primer pairs was also evaluated prior to their synthesis by verifying the absence of unwanted features such as long stretches of the same nucleotide and a high proportion of G or C residues at the 3′ end (Persing, 1993, Diagnostic Molecular Microbiology: Principles and Applications, American Society for Microbiology, Washington, D.C. 88-109). Oligonucleotide amplification primers were synthesized using an automated DNA synthesizer (Applied Biosystems).

The oligonucleotide sequence of primers or probes may be derived from either strand of the duplex DNA. The primers or probes may consist of the bases A, G, C, or T or analogs and they may be degenerated at one or more chosen nucleotide position(s) (Nichols et al., 1994, Nature. 369:492-493). Primers and probes may also consist of nucleotide analogs such as Locked Nucleic Acids (LNA) (Koskhin et al., 1998, Tetrahedron Lett. 54:3607-3630), and Peptide Nucleic Acids (PNA) (Egholm et al., 1993, Nature. 365:566-568). The primers or probes may be of any suitable length and may be selected anywhere within the DNA sequences from proprietary fragments, or from selected database sequences which are suitable for the detection of hMPV.

Variants for a given target microbial gene are naturally occurring and are attributable to sequence variation within that gene. For example, different strains of the same viral species may have a single or more nucleotide variation(s) at the oligonucleotide hybridization site. The person skilled in the art is well aware of the existence of variant nucleic acids and/or sequences for a specific gene and that the frequency of sequence variations depends on the selective pressure during evolution on a given gene product. The detection of a variant sequence for a region between two PCR primers may be demonstrated by sequencing the amplification product. In order to show the presence of sequence variations at the primer hybridization site, one has to amplify a larger DNA target with PCR primers outside that hybridization site. Sequencing of this larger fragment will allow the detection of sequence variation at this primer hybridization site. A similar strategy may be applied to show variations at the hybridization site of a probe. Insofar as the divergence of the target nucleic acids and/or sequences or a part thereof does not affect significantly the sensitivity and/or specificity and/or ubiquity of the amplification primers or probes, variant microbial DNA is under the scope of this invention. Variants of the selected primers or probes may also be used to amplify or hybridize to a variant target DNA.

In a further embodiment of the invention the diagnostic method is designed at performing simultaneous detection of nucleic acids from a variety of respiratory viruses using specific primers and/or probes. The method is aimed at detecting and/or quantifying conserved viral sequences from respiratory viruses for diagnostic purposes. Target nucleotide sequences were selected public databases and from viral strain sequences obtained in the inventor's laboratory.

The specific respiratory viral pathogens targeted by the diagnostic assays include all genotypes of influenza A and B viruses, hRSV, hMPV, PIV, adenoviruses, rhinoviruses, and enteroviruses. Other less common (coronaviruses, reoviruses) and yet to discover respiratory viruses are also targets under the scope of the present invention.

The specific combination of oligonucleotides (primers and/or probes) in a multiplex format is also another object of this invention.

Composition of matters such as diagnostic kits comprising amplification primers and/or probes for the detection of respiratory viruses are also objects of the present invention.

Amplification of Viral Sequences

In one embodiment, a PCR protocol is used for nucleic acid amplification. Multiplex assay, aimed at amplifying a plurality of respiratory viruses in the same physical enclosure, is an object of the present invention.

It has been aimed at developing DNA-based tests or diagnostic kits to detect and identify most respiratory viruses in a limited number of amplification reactions using specific primers (Table 10) and/or probes (Table 11). Although some of the primer sequences used for amplification of the various respiratory viruses, with the exception of hMPV (SEQ. ID. Nos. 95 and 96 or 110 and 111), PIV-1 (SEQ. ID. Nos. 147 and 148), PIV-3 (SEQ. ID. Nos. 151 and 152), and adenovirus (SEQ. ID. Nos. 153), have been described previously by others (Fouchier et al., 2000, J. Clin. Microbiol. 38:4096-4101 for influenza A virus/SEQ. ID. Nos.139 and 140; Li et al., 2001, J. Clin. Microbiol. 39:696-704 for influenza B virus/SEQ. ID. Nos. 141 and 142; Mazulli et al., 1999. J. Infect. Dis. 180:1686-1689 for hRSV/SEQ. ID. Nos. 143 and 144; Osiowy et al., 1998, J. Clin. Microbiol. 36: 3149-3154 for PIV-1, PIV-3, and adenovirus/SEQ. ID. Nos. 145 and 146, 149 and 150, and 154 and 155, respectively; Atmar et al., 1993, J. Clin. Microbiol. 31: 2544-2546 for rhinovirus and enterovirus/SEQ. ID. Nos. 160 and 161) and that one influenza A primer (SEQ. ID. No. 139) is near to the one described in another patent (U.S. Pat. No. 6,015,664), the object of the present invention is to combine many sets of primers and/or probes in the same physical enclosure (multiplex format).

Briefly, viral RNA was extracted from 200 μl of naso-pharyngeal aspirates or other respiratory tract samples using the QIAamp Viral RNA Mini kit (QIAGEN, Mississauga, ON, Canada). Complementary DNA (cDNA) was then synthesized using 10 μl of the RNA preparation, 0.75 μM of random hexamer primers (Amersham Pharmacia Biotech, Baie d'Urfé, QC, Canada), 300 copies of an internal control template, and the Omniscript Reverse Transcriptase Kit (QIAGEN, Mississauga, ON, Canada) following the manufacturer's instructions. The internal control template consisted of a 554 or 558 bp (depending on the multiplex assay) transcribed region of an herpes simplex virus type 2 DNA polymerase region flanked by either influenza B or rhinovirus/enterovirus complementary primer sequences (depending on the multiplex assay) cloned in the pDrive plasmid (QIAGEN).

For DNA amplification by the widely used PCR method, primer pairs were mostly derived from public database sequences (Table 10). During DNA amplification by PCR, two oligonucleotide primers binding respectively to each strand of the heat-denatured target DNA from the viral genome are used to amplify exponentially in vitro the target DNA by successive thermal cycles allowing denaturation of the DNA, annealing of the primers and synthesis of new targets at each cycle (Persing, 1993, Diagnosis Molecular Microbiology: Principles and Applications, American Society for Microbiology, Washington, D.C., 88-109).

In one embodiment, cDNA is amplified using a real-time PCR procedure with the LC Faststart DNA Master SYBR Green 1 Kit (Roche Diagnostics, Laval, QC, Canada) or the SYBR Green TAQ ReadyMix for quantitative PCR capillary formulation (Sigma-Aldrich, Oakville, ON) in a LightCycler instrument (Roche Diagnostics). Each reaction had a total volume of 20 μl including 2 μl of cDNA and 18 μl of a reaction mixture containing 4 mM MgCl₂, 2 μl of LC Faststart DNA SYBR Green 1 Master Mix or the SYBR Green TAQ ReadyMix for quantitative PCR capillary formulation, 3% DMSO, and 0.3-1.0 μM of each viral primer (see Tables 10, 12, and 13 for primer sequence ID NOs., length of amplicons, and specific combination of primers, respectively). Cycling conditions typically included an initial denaturation step of 10 min at 94° C., followed by 50 cycles of 15 s at 94° C., 5 s at 58° C., and 25 s at 72° C. However, the cycling conditions could vary slightly according to the multiplex PCR assay.

In one embodiment, two real-time multiplex PCR assays were used to amplify and detect the panel of common respiratory viruses (Table 13). The respiratory-1 multiplex assay combines 4 sets of primers for amplification of influenza A and B viruses, hRSV, hMPV, and an internal control template. The respiratory-2 multiplex assay comprises 4 other sets of primers for amplification of PIV-1, PIV-3, adenoviruses, rhinoviruses/enteroviruses, and an internal control template. The use of any other combinations of the same or similar primer sequences in two or more than two multiplex assays for detection of common respiratory viral pathogens is also under the scope of this invention.

The person skilled in the art of nucleic acid amplification knows the existence of other rapid amplification procedures such as ligase chain reaction (LCR), reverse transcriptase PCR (RT-PCR), solid phase PCR, transcription-mediated amplification (TMA), self-sustained sequence replication (3SR), nucleic acid sequence-based amplification (NASBA), strand displacement amplification (SDA), branched DNA (bDNA), cycling probe technology (CPT), solid phase amplification (SPA), rolling circle amplification technology (RCA), solid phase RCA, anchored SDA, and nuclease dependent signal amplification (NDSA) (Persing, 1993, Diagnosis Molecular Microbiology: Principles and Applications, American Society for Microbiology, Washington, D.C., 88-109; Lee, 1997, Nucleic Acid Amplification Technologies: Application to Disease Diagnosis, Eaton Publishing, Boston, Mass., 61-125; Westin et al., 2000, Nat. Biotechnol. 18:199-204).

The scope of this invention is not limited to the use of amplification by PCR, but rather includes the use of any nucleic acid amplification method or any other procedure which may be used to increase the sensitivity and/or the rapidity of nucleic acid-based diagnostic tests. The scope of the present invention also covers the use of any nucleic acids amplification and detection technology including real-time or post-amplification detection technologies, any amplification technology combined with detection, any hybridization nucleic acid chips or array technologies, any amplification chips or combination of amplification and hybridization chip technologies. Detection and identification by any nucleotide sequencing method is also under the scope of the present invention.

Detection of Amplification Products

Classically, the detection of PCR amplification products is performed by standard ethidium bromide-stained agarose gel electrophoresis. Using this approach, the amplicons may be discriminated based on their size. In the present invention, the length of the different amplicons varies from 84 to 929 bp (Table 12). It is however clear that other detection methods of amplified products may be faster, more sensitive, and more practical. Amplicon detection may also be performed by solid support or liquid hybridization using strain-specific internal DNA probes hybridizing to an amplification product. Such probes may be generated from sequences designed to specifically hybridize to DNA amplification products which are objects of the present invention. Amplicons can be also characterized by DNA sequencing. Alternatively, a rapid detection method developed with real-time PCR assays consist of determining the specific melting temperature of amplicons at the end of the amplification reaction.

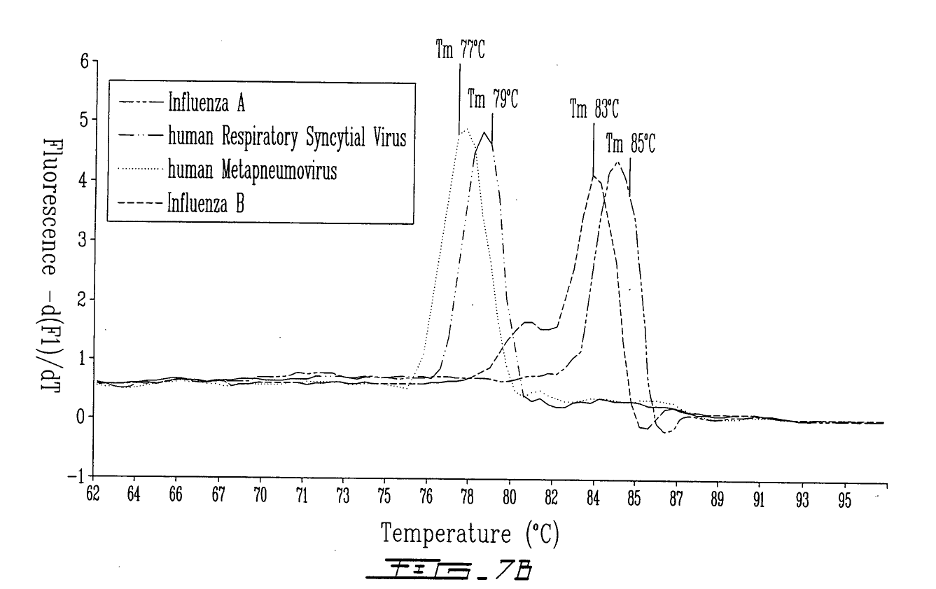

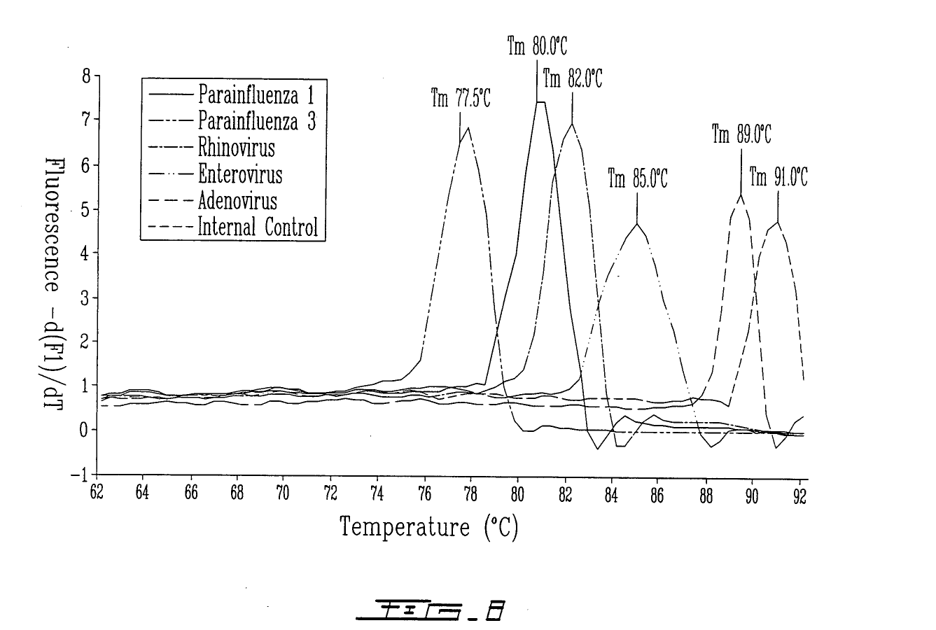

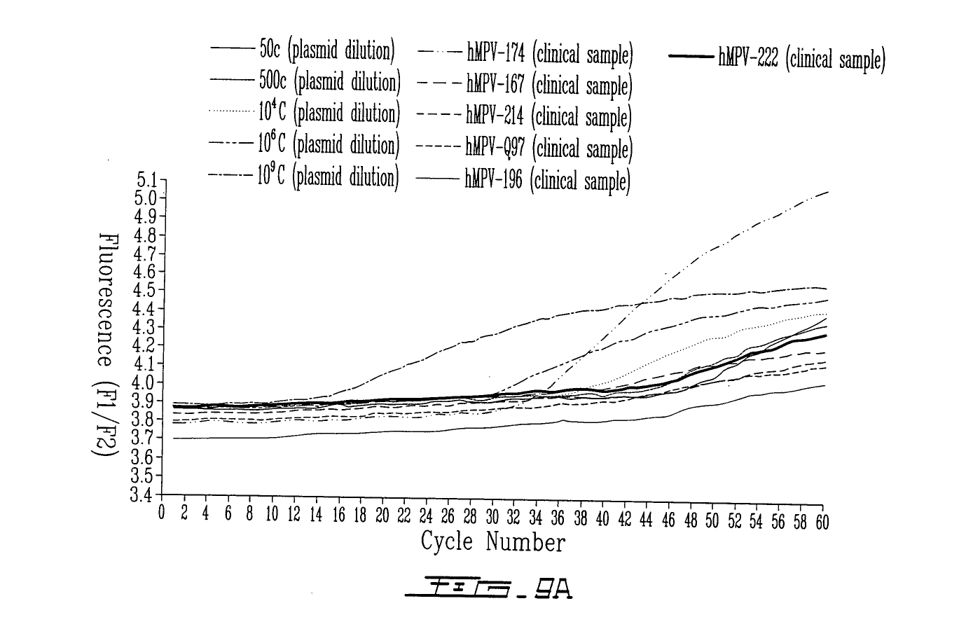

The detection of viral amplicons was first characterized using the melting curve analysis feature of a real-time PCR instrument. Using this procedure, the different viral targets amplified in each of the multiplex PCR assays could by differentiated by their specific melting temperature (Table 13 and FIGS. 7 and 8). Alternatively, specific fluorescent probes (Table 11) for each viral target can be used for real-time detection and quantification of amplicons in a single amplification/detection assay. Real-time quantification of viral products can be performed through a variety of fluorescence-based technologies such as adjacent probes, Taqman probes, and molecular beacons. Alternatively, these probes may be used in a separate post-amplification step.

The diagnostic kits, primers and probes mentioned above can be used to detect and/or identify respiratory viruses, whether said diagnostic kits, primers and/or probes are used for in vitro or in situ applications. The said samples may include but are not limited to: any clinical sample, any environmental sample, any viral culture, any tissue, and any cell line.

In the methods and kits described herein, the oligonucleotide probes and amplification primers have been derived from larger sequences (i.e. DNA fragments of at least 100 base pairs). It is clear to the individual skilled in the art that oligonucleotide sequences other than those described in the present invention and which are appropriate for detection and/or identification of any respiratory viruses may also be derived from those selected in the present invention. For example, the oligonucleotide primers or probes may be shorter but of a length of at least 10 nucleotides or longer than the ones chosen; they may also be variants of the same oligonucleotide. If the target DNA or a variant thereof hybridizes to a given oligonucleotide, or if the target DNA or a variant thereof can be amplified by a given oligonucleotide PCR primer pair, the converse is also true; a given target DNA may hybridize to a variant oligonucleotide probe or be amplified by variant oligonucleotide PCR primers. Alternatively, the oligonucleotides may be designed for use in amplification methods other than PCR. Consequently, one aspect of the present invention is the simultaneous detection and/or identification of various respiratory viruses by targeting nucleic acid sequences which are used as a source of specific and ubiquitous oligonucleotide probes and/or amplification primers. Although the selection and evaluation of oligonucleotides suitable for diagnostic purposes require much effort, it is quite possible for the individual skilled in the art to derive, from the selected oligonucleotides (listed in Tables 10 and 11), other oligonucleotides which are suitable for diagnostic purposes.

Thus, the specific primers and/or probes selected for amplification and detection of respiratory viruses and their specific combination in multiplex assays are objects of this invention (Tables 10-13).

The present invention will be more readily understood by referring to the following examples which are given to illustrate the invention rather than to limit its scope.

### EXAMPLE I

### Evaluation of Different Primers for hMPV Detection

An evaluation of the sensitivity of different hMPV primer sets in a real-time PCR assay was performed using 20 positive hMPV cultures grown in LLC-MK2 cells and 10 naso-pharyngeal aspirate specimens. These samples were collected in the Quebec City area (QC, Canada) over a period of 3 years (2000-2002). Viral RNA was prealably extracted from 200 μl of viral culture supernatants or naso-pharyngeal aspirates using the QIAamp Viral RNA Mini Kit (QIAGEN). Complementary DNA was synthesized using 10 μl of eluted RNA, 0.75 μM of a specific hMPV primer (Table 2, sequences ID Nos. 91, 97, 100, 102, 112), and the Omniscript Reverse Transcriptase Kit (QIAGEN, Mississauga, ON, Canada) following the manufacturer's recommendations. Complementary DNA was amplified using a real-time PCR procedure with the LC Faststart DNA Master SYBR Green 1 Kit (Roche Diagnostics, Laval, QC, Canada) in a LightCycler instrument (Roche Diagnostics). Each reaction had a total volume of 20 μl including 2 μl of RT mixture and 18 μl of a reaction mixture containing 4 mM MgCl₂, 2 μl of Faststart DNA SYBR Green Master Mix, 3% DMSO, and 0.5 μM of each hMPV primer (Table 4: sequence ID Nos.: 92 and 94; 100 and 101; 97 and 98; 102 and 103; and 112 and 113, for the nucleoprotein (N), matrix (M), fusion (F), phosphoprotein (P), and polymerase (L) genes respectively). Cycling conditions typically included an initial denaturation step of 10 min. at 94° C., followed by 50 cycles of 15 s at 94° C., 5 s at 54° C., and 30 s at 72° C. However, the cycling conditions could slightly vary according to the hMPV gene to amplify. Detection of hMPV amplicons was characterized using the melting curve analysis feature of a real-time PCR instrument. Briefly, following the last amplification cycle on a LightCycler instrument, the internal temperature was rapidly increased to 94° C. then decreased to 60° C. for 30 seconds, followed by slowly increasing to 94° C. at a rate of 0.1° C. per second, with continuous fluorescence lecture.

Using this procedure, the specific melting temperature obtained with the hMPV amplicons produced by the primers specific for target genes coding for N, M, F, P and L proteins was found to be 82.63° C.±0.87° C., 82.75° C.±0.80° C., 83.29° C.±0.45° C., 80.48° C.±0.46° C., and 77.99±0.33° C., respectively. As shown in Table 5, a real-time PCR assay using primers complementary to the N gene successfully amplified RNA from all 20 cultures and 10 clinical specimens. In contrast, HMPV primers specific for the M, F, P, and L genes had sensitivities of only 70%, 63.3%, 50%, and 89.7% respectively. Thus, primers aimed at amplifying the hMPV N gene appear to target more conserved regions of the hMPV genome and, consequently, were selected for further detailed evaluation (see example 2).

### EXAMPLE II

### Validation of a Real-Time PCR Assay for the hMPV Nucleoprotein (N) Gene in a Pediatric Population

An hMPV N plasmid was constructed by subcloning the amplified hMPV N region in the plasmid pDrive (QIAGEN, Mississauga, ON, Canada). The new plasmid was transcribed using the RNA Transcription Kit (Stratagene, Vancouver, BC, Canada) for sensitivity analysis. Using the previously described RT procedure (N primer, sequence ID No. 91) and real-time PCR protocol (N primers, sequence ID Nos. 92 and 94) reported in example 1, the sensitivity of the assay was estimated at 100 copies per reaction with a specific melting temperature of 82° C. The assay was found to be specific with no amplification signal observed when viral cultures positive for the human respiratory syncytial virus, the parainfluenza viruses, the influenza viruses A and B, and the adenoviruses were tested. The real-time PCR assay for the hMPV N gene was subsequently validated in a prospective case-control study in children below the age of 3 years hospitalized at Laval University Hospital Center (CHUQ-CHUL) in Quebec City, Canada, during the winter season of 2001-O₂. Cases consisted of children hospitalized for a respiratory syndrome whereas controls consisted of children hospitalized during the same period for a surgery with no respiratory symptoms. At admission, a nasopharyngeal aspirate was collected for viral antigen tests, viral cultures, and RT-PCR studies for influenza A and B viruses, the human respiratory syncytial virus (hRSV), and the hMPV N gene.

No virus was detected in any of the samples from the 51 control children. Among the 208 cases tested by real-time PCR for hMPV, hRSV, influenza A and B viruses, the positivity rates were 5.8%, 51.0%, 21.6%, and 0%, respectively. Viral culture was positive for hMPV in only 2 cases whereas the real-time hMPV PCR assay for the N gene was positive for 12 cases including the 2 positive by viral culture. All positive hMPV PCR results for the N gene were confirmed by a second PCR assay targeting the hMPV fusion (F) gene and by the use of DNA sequencing which indicated the presence of multiple hMPV strains clustering in the two previously-reported F genotypes. Thus, the real-time PCR assay describes in this invention appears to be much more sensitive than conventional viral culture for the detection of hMPV in clinical samples.

### EXAMPLE III

### Typing of hMPV Clinical Strains Using Restriction Enzyme Analysis

A rapid molecular typing method has been designed for hMPV epidemiological studies based on amplification of a portion of the fusion (F) gene which is then digested with specific restriction enzymes. Briefly, a 759-bp fragment of the hMPV F gene was amplified by a real-time PCR procedure as described in example 1 using specific primers (sequence ID Nos. 97 and 98). After PCR amplification, two aliquots (10 uL) of the PCR product were digested during 2 hours at 37° C. with 10 units of ApaL 1 and 4 units of Avr II (New England Biolabs, Mississauga, ON, Canada) according to the manufacturer's recommendations. Digested products were then loaded on an 1% agarose gel for electrophoresis.

Table 6 summarizes the size of the F fragments obtained for different hMPV isolates following enzymatic digestion and the corresponding genotype as defined by DNA sequencing and phylogenetic studies. In brief, all hMPV isolates belonging to the genotype F-I were digested by ApaL I (fragments of 532 and 227 bp) but not by Avr II whereas those belonging to the genotype F-II were digested by Avr II (fragments of 447 and 312 bp) but not by ApaL I (see FIG. 2 for an example of the band pattern following electrophoresis on an agarose gel).

### EXAMPLE IV

### A Prospective Case-Control Study for Defining the Role of Respiratory Viruses in Children Hospitalized for Acute Respiratory Tract Infections

### SUMMARY

The human metapneumovirus (hMPV) is a new paramyxovirus recently isolated from young children with respiratory syndromes. The objectives of this study were to estimate the relative contribution of hMPV in children's hospitalization for acute respiratory tract infections (ARTI) and to define its clinical presentation and seasonal pattern.

The percentage of hospitalizations has been assessed for ARTI in children aged 0-3 years attribuate to hMPV and other respiratory viruses in a prospective case-control study during the winter/spring seasons of 2002. Multiplex real-time PCR assays and other conventional diagnostic methods were used to detect hMPV, the human respiratory syncytial virus (hRSV), and the influenza viruses in nasopharyngeal aspirates of children.

HMPV was detected in 12 (5.8%) of the 208 children hospitalized for ARTI as compared to 118 (56.7%) for hRSV and 49 (23.6%) for influenza A. In contrast, none of the 51 controls harbored any of the respiratory viruses. Most hRSV and influenza infections occurred in January and February whereas the peak hMPV activity was in March and April. The peak age for hospitalizations for hRSV and hMPV infections was <3 months and 3-5 months, respectively. Diagnoses of bronchiolitis and pneumonitis were made in respectively 67% and 17% of hMPV-infected children compared to 84% and 25% for those with hRSV infection. None of the hMPV-infected children was admitted to the intensive care unit compared to 15% for those with hRSV or influenza A infections.

HMPV causes significant morbidity in young children and its clinical presentation is similar although less severe than that of hRSV.

Introduction

The human metapneumovirus (hMPV) is the first human member of the new Metapneumovirus genus within the Paramyxoviridae family (van den Hoogen B G, de Jong J C, Groen J, et al. Nat Med 2001;7:719-24; van den Hoogen B G et al. Virology 2002;295:119-32.) The human respiratory syncytial virus (hRSV) belongs to a separate genus within the same family (Taxonomy V. Seventh Report of the International Committee on Taxonomy of Viruses. Academic, San Diego, 2000.).

HMPV has been recently identified in nasopharyngeal aspirates of children and adults suffering from a range of respiratory tract infections in various parts of the world (van den Hoogen B G, de Jong J C, Groen J, et al. Nat Med 2001;7:719-24; Nissen M D, et al. Med J Aust 2002;176:188; Peret T C, Boivin G, Li Y, et al. J Infect Dis 2002;185:1660-3; Stockton J, et al. Emerg Infect Dis 2002;8:897-901). The clinical syndrome of the infected children encompasses mild respiratory problems to bronchiolitis and pneumonitis (van den Hoogen B G, de Jong J C, Groen J, et al. Nat Med 2001;7:719-24; Pelletier G, et al. Emerg Infect Dis 2002;8:976-8; Boivin G, Abed Y, Pelletier G, et al. J Infect Dis 2002; 186:1330-4).

Using reverse-transcription polymerase chain reaction (RT-PCR), the rate of hMPV detection in nasopharyngeal aspirates (NPA) of pediatric patients with unexplained ARTI varied between 1.5 and 10% (van den Hoogen B G, de Jong J C, Groen J, et al. Nat Med 2001;7:719-24; Nissen M D, et al. Med J Aust 2002; 176:188). These retrospective studies are small, excluded patients who tested positive for other viruses, only superficially describe the clinical features of the disease and lack morbidity/mortality data. In absence of a control group, these studies cannot differentiate if hMPV is a colonizing or a pathogenic virus. More recently, Stockton et al. identified hMPV RNA in 2.2% of 405 specimens from patients with influenza-like illnesses who consulted general practitioners in England although few swabs were collected from children <5 years (Stockton J, et al. Emerg Infect Dis 2002;8:897-901).

The objectives of this study were to estimate the relative contribution of hMPV in children's hospitalization for acute respiratory tract infections (ARTI) and to define its clinical presentation and seasonal pattern.

Materials and Methods

Study design. Participants were children aged ≦3 years hospitalized between Dec. 15, 2001 and Apr. 20, 2002 at Laval University Hospital Center (CHUQ-CHUL) in Québec City, Canada. Cases were admitted for an ARTI (bronchiolitis, pneumonitis, laryngotracheobronchitis, etc. . . . ) whereas controls were hospitalized for an elective surgery and had no respiratory symptoms or fever. Children hospitalized twice were counted as two cases. At admission, after signed consent was obtained from their parents, all children had an NPA (1-2 ml) collected and tested for hRSV antigen (TestPack, Abbott Laboratories, Abbott Park, Ill.). The specimen was frozen at −80° C. until subsequent RT-PCR studies. Additionally, viral cultures, direct immunofluorescence staining, and microbiological testing were performed according to the treating physician. A standardized questionnaire was completed at admission by a single research nurse with the parents. At the end of the hospitalization, the children's charts were reviewed to collect clinical and laboratory data. The study was approved by the CHUQ-CHUL research ethics board.

RNA extraction and cDNA synthesis. Viral RNA was extracted from 200 μl of NPA specimens using the QIAamp viral RNA Mini Kit (QIAGEN, Mississauga, ON, Canada). Complementary cDNA was synthesized using 10 μl of eluted RNA and the Omniscript Reverse Transcriptase kit (QIAGEN). Random hexamer primers (Amersham Pharmacia Biotech, Baie d'Urfé, QC, Canada) were used in the RT step of the multiplex respiratory PCR assay whereas a specific primer (5-TGGGACAAGTGAAAATGTC-3) serves to synthesize hMPV cDNA in a separate PCR assay. An internal control (300 copies) consisting of a 534-bp transcribed region of the pDrive plasmid (QIAGEN) flanked by influenza B complementary primer sequences (see below) was spiked in the RT reaction of the multiplex respiratory PCR assay to verify the presence of PCR inhibitors.

RT-PCR assays and phylogenetic studies. The multiplex respiratory PCR assay was designed to amplify conserved regions of the influenza A (SEQ. ID Nos.139-140) (Fouchier R A, et al. J Clin Microbiol 2000; 38:4096-101) and B (SEQ. ID Nos. 141-142) (Li J, Chen S, Evans D H. J Clin Microbiol 2001; 39:696-704) matrix genes as well as the fusion gene of hRSV (SEQ. ID Nos. 143-144) (Mazzulli T, Peret T C, McGeer A, et al. J Infect Dis 1999; 180:1686-9).

Complementary DNA was amplified using a real-time PCR procedure with the LC Faststart DNA Master SYBR Green 1 Kit (Roche Diagnostics, Laval, Québec, Canada) in a LightCycler instrument (Roche Diagnostics). Each reaction had a total volume of 20 μl including 2 μl of cDNA and 18 μl of a reaction mixture containing 2.4 mM MgCl₂, 2 μl of Faststart DNA SYBR Green Master Mix, 3% DMSO, 0.3 mM each of influenza A and B primers, and 0.8 mM of hRSV primers. The thermal cycling incubations consisted of an initial denaturation step at 94° C. for 10 min, followed by 50 cycles of 15 s at 940, 5 s at 58° C., and 25 s at 72° C. At the end of each cycle, the fluorescent signal was measured at a wavelength of 530 nM using the LightCycler Fluorimeter. The melting curve analysis program of the LightCycler was used to identify specific PCR products of influenza A (Tm: 85° C.), influenza B (Tm: 83° C.), hRSV (Tm: 79° C.), and the internal control (Tm: 92° C.). The sensitivity of the multiplex respiratory PCR assay was estimated at 50 copies for each viral target when mixed with 300 copies of the internal control.

A specific PCR assay was designed for amplification of the hMPV Nucleoprotein (N) gene in the LightCycler instrument. The sequences of the forward (MPV-NCS) and reverse (MPV-NCA) primers were respectively 5′-GAGTCTCAGTACACAATTAA-3 (SEQ ID NO 92) and 5-GCATTTCCGAGAACAACAC-3 (SEQ ID NO 94). The specific cDNA was amplified using conditions similar to those of the multiplex respiratory PCR assay except that 0.5 mM of each primer was added to the master mix. Cycling conditions included an initial denaturation step of 10 min at 94° C., followed by 50 cycles of 15 s at 94° C., 5 s at 54° C., and 30 s at 72° C. The melting temperature of the amplified hMPV N fragment was 82° C. and the sensitivity of the PCR assay was 100 copies. For phylogenetic studies, nucleotide sequences were determined from amplified hMPV F (fusion) gene products then analyzed using the neighbor joining algorithm and Kimura-2 parameters. (Boivin G, Abed Y, Pelletier G, et al. J Infect Dis 2002; 186:1330-4).
hMPV in the general population. To further assess the seasonal distribution, affected age groups and frequency of hMPV, we compared data from the study to those of the general population derived from the viral cultures found positive for hMPV in our regional virology diagnostic laboratory, the only one performing viral cultures for the Québec City area (population approximately 600 000). Respiratory specimens were inoculated in 96-well plates containing ten cell lines (MDCK, LLC-MK2, Hep-2, human foreskin fibroblast, Vero, mink lung, A-549, rhabdomyosarcoma, 293, and HT-29) then incubated for a period of 21 days. A positive cytopathic effect was confirmed by immunofluorescence testing using monoclonal antibodies or by RT-PCR (hMPV).

Statistical analyses. Proportions were compared by the χ² test.

Results

The study population included a total of 208 hospitalized cases with ARTI (including 8 children who were admitted twice) and 51 children who served as controls.

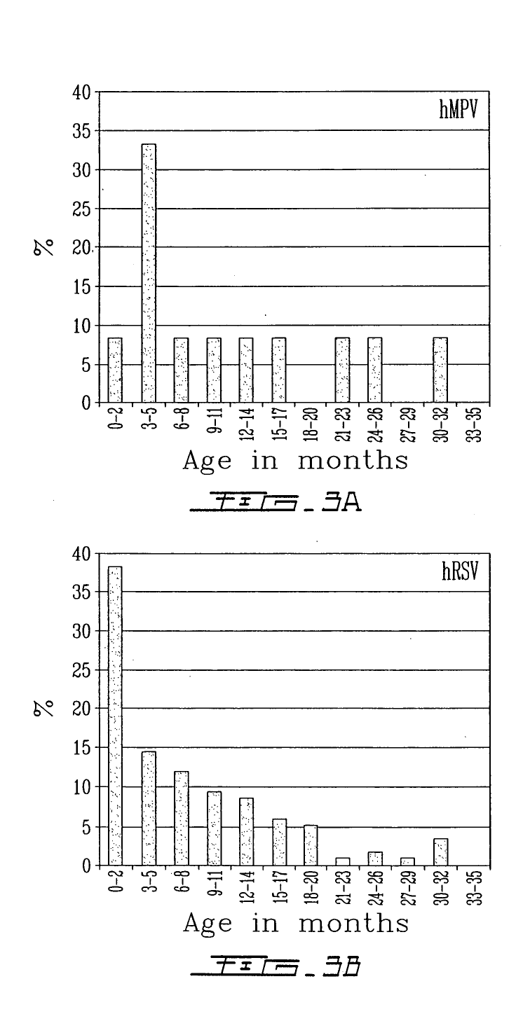

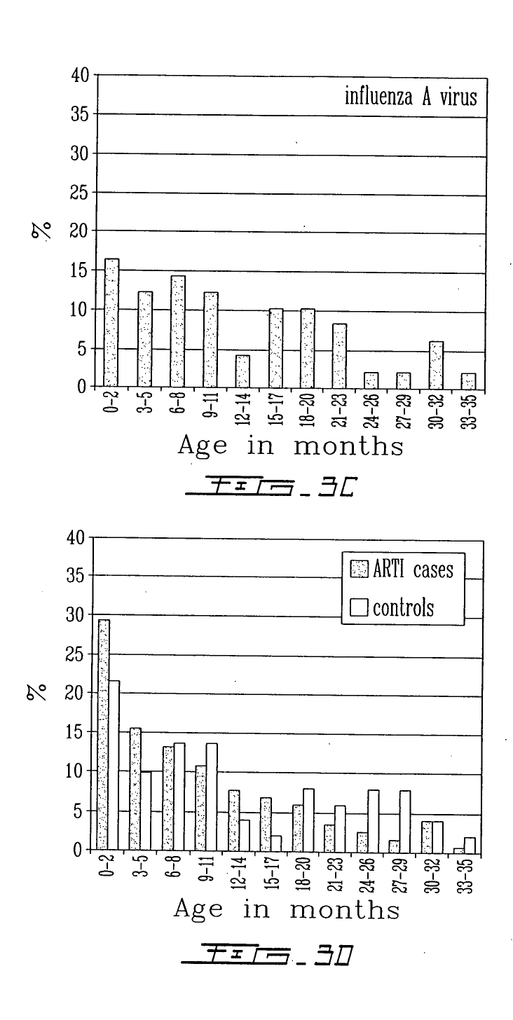

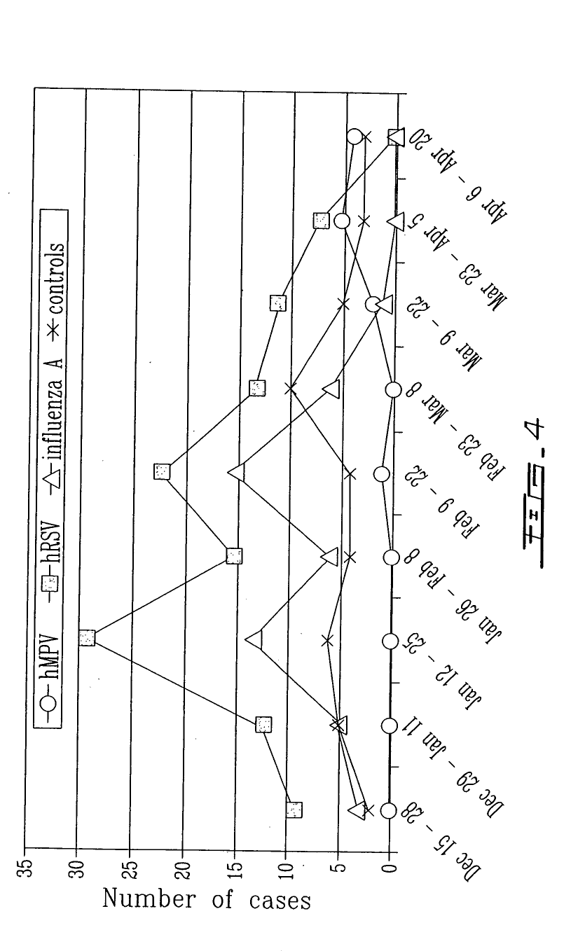

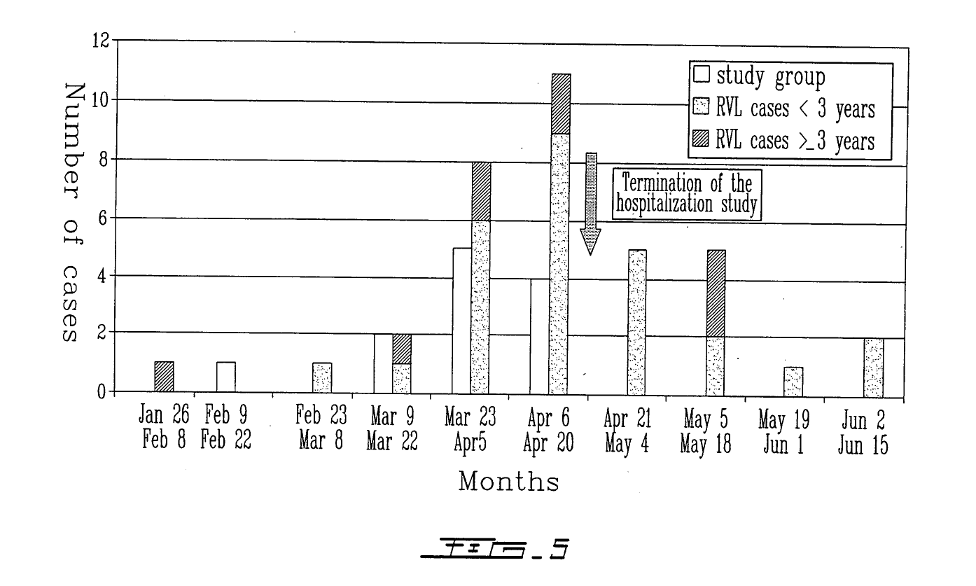

Description of the study population and viral etiologies. The age distribution of children with ARTI is presented in FIG. 3: 56% (116) were male and 44% (92) were female. Hospitalization was the highest in infants ≦3 months of age and steadily decreased thereafter. Most (90%) children had no underlying medical conditions at admission.

All 208 cases had an NPA. The mean delay between the onset of symptoms and collection of NPA was 6 days (median 4 days). This delay did not differ for the different viruses detected. All 208 cases were tested by PCR, 204 had an hRSV antigen detection test, 100 had a direct examination procedure for other viruses, and 100 had a viral culture. At least one respiratory virus was detected in 164 (79%) cases whereas none was detected in 44. Twelve children (5.8%) were positive for hMPV, 118 (56.7%) for hRSV, 49 (23.6%) for influenza A, and none for influenza B (Table 7). There was no PCR testing for adenoviruses and PIV type 2 but these two viruses were found in 6% and 2%, respectively, of the 100 viral cultures or antigenic assays. Single virus infections occurred in 141 (86%) of the 164 positive cases and mixed infection was present in 23 (14%). Two of the 12 hMPV infections were mixed (hMPV-influenza and hMPV-hRSV). The other combinations were hRSV-influenza A (18 cases), hRSV-adenovirus (2), and influenza A-adenovirus (1).

Among the 208 cases tested by PCR for hMPV, hRSV, influenza A and B viruses, the positivity rates were 5.8%, 51.0%, 21.6%, and 0%, respectively (Table 7). In addition, 16 other cases had one of the four latter respiratory viruses identified only by culture (3 influenza A and one hRSV), only by an antigen detection test (10 hRSV and one influenza A) or by both culture and antigen detection test (I hRSV). Among the 8 children hospitalized twice, none had the same viral infection at both admissions. The combinations observed were hMPV—hRSV (2), hMPV—no virus (1), hRSV—no virus (2), hRSV—influenza (2), no virus—no virus (1).

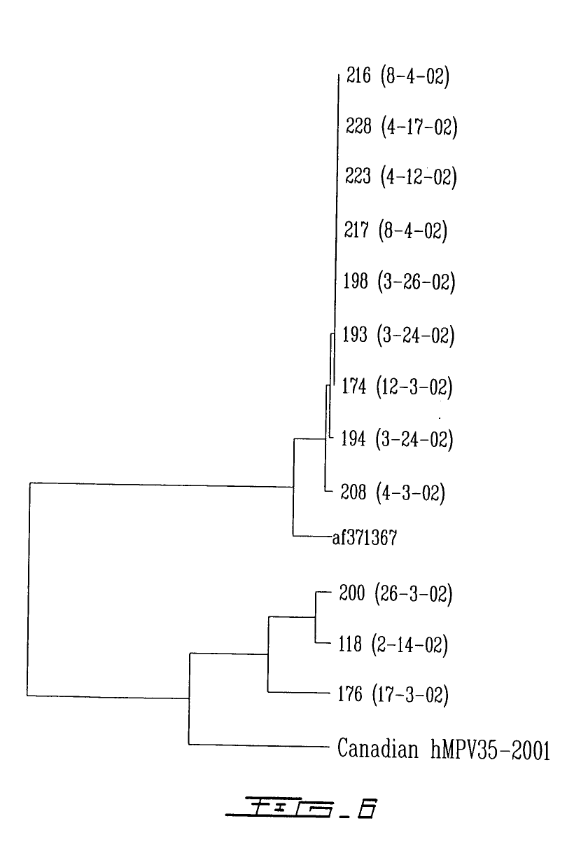

The biweekly distribution of cases with respiratory tract viruses is shown in FIG. 4. hRSV and influenza A infections occurred predominantly between January and March, whereas hMPV infections occurred mostly in March and April. The proportion of children with virologically-confirmed respiratory tract infection decreased after February.

Clinical features of cases. The distribution of the age distribution by different viral infections is shown in FIG. 3. While the peak age for hMPV infection was 3-5 months, it was before 3 months for hRSV infection. For influenza A, it spread evenly during the first year of life. The peak age for mixed infection was 6-11 months and decreased thereafter. Gender was distributed evenly with each virus group but more males (70%) had mixed viral infections. The majority (75% with hMPV, 93% with hRSV, 90% with influenza A virus infection) of the children in different etiological groups had no medical underlying conditions. Three (25%) children with hMPV infection had a cardiac disorder, one having concomitant cardiac, pulmonary, and neurological problems.

Signs and symptoms recorded with the different respiratory viruses were generally similar except for the vomiting which occurs more frequently with hMPV infections (p=0.05) (Table 8). The mean and median duration of hospitalization was similar for hMPV, hRSV and influenza A viruses being respectively 5.5 and 4.5, 5.8 and 5.0, and 4.9 and 4.0 days. Of note, one third of hMPV-infected children were hospitalized more than 7 days. None of the children with hMPV infection was admitted to the intensive care unit (ICU) contrasting with 15% and 16% of children with hRSV and influenza A, respectively. There was no death as part of this study. The duration of the hospitalization in children with no detectable virus was shorter than that in children with single or mixed infection (Kruskal-Wallis Test, p<0.0001). Two-thirds of the children were given antibiotics during their hospitalization, independently of the etiology of their respiratory disease.

At hospital discharge, a final diagnosis of bronchiolitis was given to 67% of children with hMPV, 84% with hRSV, and 51% with influenza A (p<0.001) (Table 9). Otitis occurred in about half of children with hMPV, hRSV, and influenza A infections. Pneumonitis was less frequently diagnosed in children with hMPV compared to those with hRSV or influenza A (17%, 25%, and 37%, p=0.22). Final diagnoses were similar with single and mixed infections.

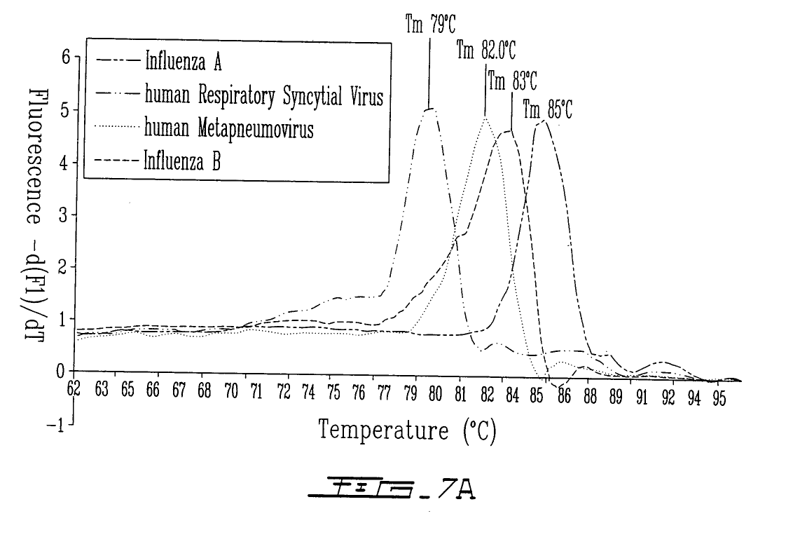

Controls. There were 51 children in the control group, 29 (56.9%) males and 22 (43.1%) females. The age distribution of the controls was similar to that of the cases (FIG. 3) as was the period of hospital admission (FIG. 4). None of the controls had a virus detected by PCR.
hMPV in the general population. The regional virology laboratory received 1505 respiratory specimens for viral culture between Jan. 1 and Jun. 30, 2002. In total, 36 (including two study participants) or 2.9% were positive for hMPV: 24 (66.6%) in children <2 years of age, 5 in those 2 to 4 years, 4 in adults 30-49 years, and 3 in those 70 years or older. Most cases (81%) were isolated over a two-month period (from March 23 to May 18) (FIG. 5). When comparing the seasonal distribution of hMPV in hospitalized children (study population) and in the general population, it appears that the study in hospitalized children did not cover the entire hMPV season as it was stopped just after the peak time of hMPV transmission (April 6-20).

Phylogenetic analyses of hMPV strains. The 12 hMPV strains isolated in the prospective study clearly clustered into two F lineages with nine strains belonging to group 1 and three to group 2 (FIG. 6). Seven of the 9 group 1 strains had identical F gene sequences. At the nucleotide level, there was 84-85% similarity between groups versus 98-100% (group 1) and 93-99% (group 2) similarity within groups. There were fewer differences at the amino acid level i.e. 94-96% similarity between groups versus 99-100% (group 1) and 98-99% (group 2) among strains within the same group. Although there was a trend for more recent strains to cluster in group 2, the correlation between genotypes and date of hospitalization was not perfect as two strains detected on the same day (No. 216 and 217) belonged to two different F lineages.

Discussion

The present example has revealed important clinical and epidemiological features related to hMPV. First, the data indicate that hMPV is really a respiratory pathogen. Second, it was found that it contributes to ARTI leading to children's hospitalization in proportion similar to adenoviruses or PIV but significantly less than hRSV and influenza viruses. Third, the symptoms and complications associated with hMPV were indistinguishable from those caused by hRSV. Finally, the results show that the epidemiology of hMPV infection in children can differ from that of hRSV and influenza viruses both in terms of the age of first acquisition and in the timing of the seasonal epidemics.

Recent studies by our group (Pelletier G, et al. Emerg Infect Dis 2002; 8:976-8; Boivin G, Abed Y, Pelletier G, et al. J Infect Dis 2002; 186:1330-4) and others (van den Hoogen B G, de Jong J C, Groen J, et al Nat Med 2001; 7:719-24; Nissen M D et al. Med J Aust 2002; 176:188; Stockton J, et al. Emerg Infect Dis 2002; 8:897-901) had showed that hMPV should be added to the list of human respiratory viral pathogens (Dowell S F, Anderson L J, Gary H E, Jr., et al. J Infect Dis 1996; 174:456-62; Treanor J J. Respiratory Infections. In: Richman D D, Whitley, R. J., Hayden, F. G., ed. Clinical Virology. Vol. 1. Washington: ASM Press, 2002:7-26).

The absence of hMPV in respiratory tract of our 51 control children, the absence of other respiratory viruses in 83% of the 12 hMPV-infected children and the severity of their symptoms (bronchiolitis and/or pneumonitis) provide further evidence that hMPV is really a pathogenic respiratory virus. The absence of underlying medical conditions in 75% of the hMPV-infected children further demonstrates its pathogenicity. The use of PCR assays was particularly advantageous for hMPV because its growth in culture is fastidious and limited to a few permissive cell lines (Boivin G, Abed Y, Pelletier G, et al. J Infect Dis 2002; 186:1330-4) and because rapid antigenic detection tests are currently unavailable.

The present example provides for the first an estimate of the proportion of ARTI hospitalizations attributable to hMPV in a pediatric population. During the period ranging from Dec. 15 to Apr. 20, 2002, hMPV accounted for 5.8% of all hospitalizations for respiratory tract infections in children aged 0-3 years. This underestimates the real proportion as our hospitalization study stopped before the end of the hMPV season prevailing in the community (FIG. 5). Although we did not perform PCR testing for adenoviruses and PIV, the percentage of hospitalizations caused by hMPV seems similar to that attributable to these two viruses and much smaller than that of hRSV or influenza A.

One important observatin of the present example is that hMPV disease cannot be distinguished on clinical findings from that caused by hRSV and influenza A (Tables 8 and 9) but tended to be somewhat less severe with fewer pneumonia and no admission in the ICU. Nevertheless, hMPV infection was associated with a significant clinical and economical burden as shown by a mean hospital stay of 5.5 days and one-third of hMPV-infected cases being hospitalized for >7 days.

A small serological study from children over the age of 5 years had hMPV antibodies showing a high level of transmission. Data from the present study show that morbidity caused by hMPV is greatest in children <2 years of age as they represented 83% (10/12) of our hospitalized cases and two-thirds of the hMPV isolates recovered in our diagnostic virology laboratory. This shows that similar to other paramyxoviruses such as hRSV, the morbidity of hMPV in children occurs through primo-infection. In contrast to hRSV which peaked during the first three months of life, hMPV infections peaked at a slightly older age i.e. between the third and fifth month of life. Whether this difference depends on a longer persistence of maternal antibodies or a less efficient transmission mode in the case of hMPV would require additional studies.

During the 4-week period from mid-March to mid-April, hMPV infections clustered (11/12 detected) and accounted for 18.9% of all hospitalizations for ARTI in children. This contrasts with hRSV and influenza A which occurred mostly in January and February (FIG. 4). Based on passive surveillance data from our regional virology laboratory, the peak time of hMPV transmission in the community occurred between Apr. 6-20, 2002, and continued beyond the end of the present study in hospitalized children (FIG. 5). Year-long active surveillance studies will be required to better assess the seasonal patterns of hMPV transmission.

As described for hRSV (Mazzulli T, Peret T C, McGeer A, et al J Infect Dis 1999; 180:1686-9), several strains of hMPV circulated during a very short period of time (one month) in the present study area. The hMPV strains segregated into two F subgroups in agreement with previous studies although one strain clearly predominated accounting for 58.3% of all isolates (van den Hoogen B G, de Jong J C, Groen J, et al. Nat Med 2001; 7:719-24; Peret T C, Boivin G, Li Y, et al. J Infect Dis 2002; 185:1660-3; Boivin G, Abed Y, Pelletier G, et al. J Infect Dis 2002; 186:1330-4).

Due to the small number of hMPV strains belonging to one of the F subgroup, we did not attempt to correlate hMPV genotype with clinical outcome. Such viral heterogeneity may allow multiple reinfections throughout life especially in elderly subjects and immunocompromised patients (Pelletier G, Dery P, Abed Y, Boivin G Emerg Infect Dis 2002; 8:976-8).

In conclusion, the present study supports the fact of the epidemic nature of hMPV infection and establishes its role as a significant pathogen in severe ARTI of children.

### EXAMPLE V

### Determination of the hMPV Load in Clinical Samples Using Quantitative Real-Time PCR

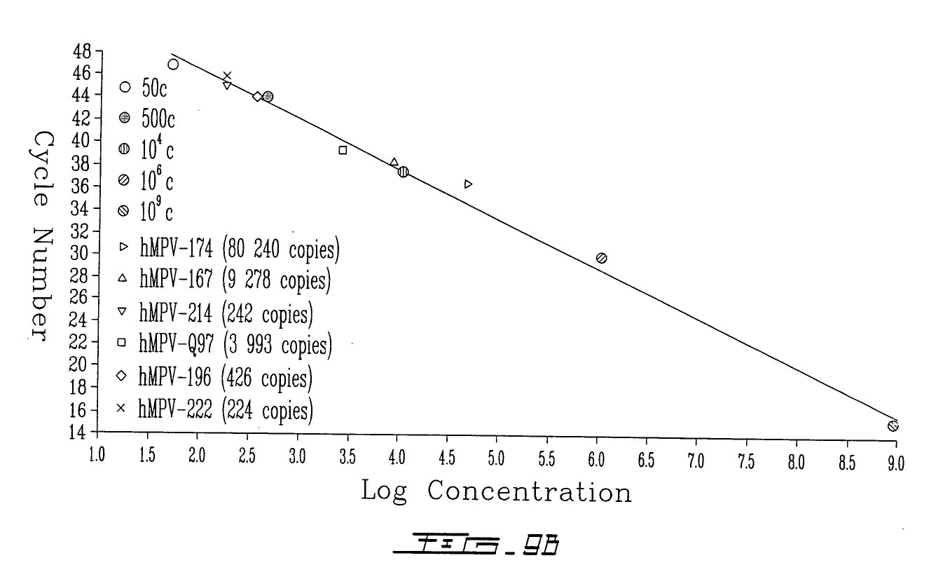

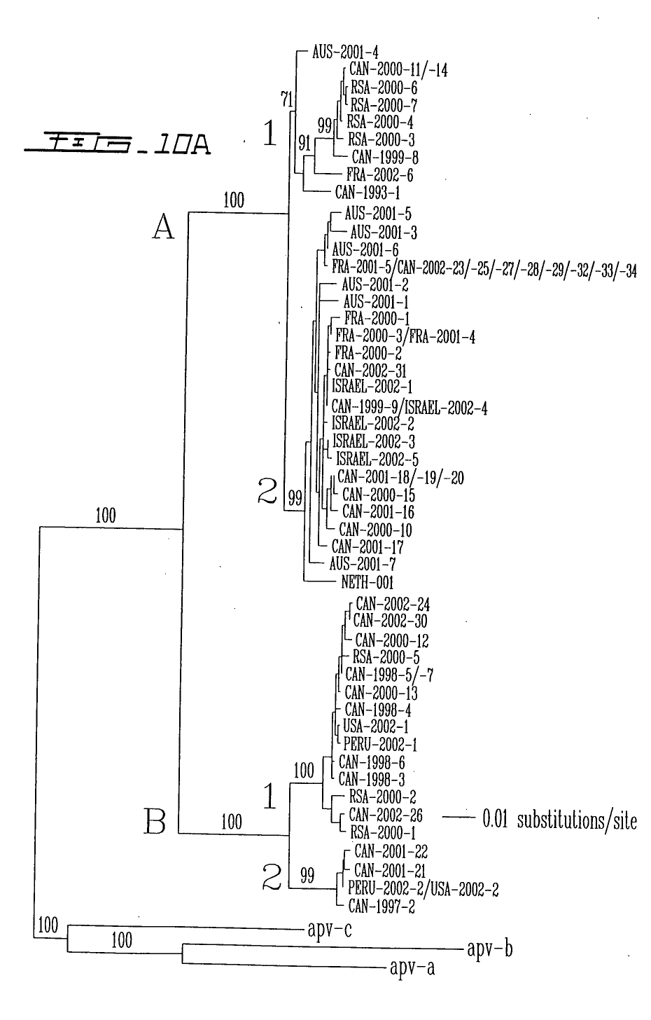

An evaluation of a quantitative real-time PCR hMPV assay using a TaqMan probe was performed using naso-pharyngeal aspirate specimens collected from children and adults in Quebec City during the winter seasons of 2001-02 and 2002-03. Viral RNA was first extracted from 200 μl of naso-pharyngeal aspirates using the QIAamp Viral RNA Mini Kit (QIAGEN) and eluted in a volume of 40 μl. Serial concentrations (5×10² to 1×10⁹ copies) of a transcribed hMPV plasmid were used to construct the standard curve of the assay. Complementary DNA was synthesized using 10 μl of eluted RNA from clinical samples or hMPV plasmid dilutions, 50 ng (0.75 μM) of random hexamer primers (Amersham Pharmacia Biotech), 0.5 mM of dNTPs, 1× Omniscript buffer and 4 units of the Omniscript reverse transcriptase (QIAGEN) in a final volume of 20 μl. The reverse transcription (RT) mixture was incubated during 1.5 hr at 37° C. and inactivated at 70° C. during 10 min. Complementary DNA was amplified by real-time PCR with a TaqMan probe for the hMPV L gene and the LC Master Hybridization Probe Mix (Roche Diagnostics) in a LightCycler instrument (Roche Diagnostics). Each reaction had a volume of 20 μl including 2 μl of RT mixture, 4 mM MgCl2, 0.3 μM of specific hMPV L primers (sequence ID Nos. 112 and 114), 0.6 μM of a specific hMPV L probe (SEQ. ID. No. 127), and 2 μl of the LC Master Hybridization Probe Mix (Roche Diagnostics). Cycling conditions included an initial denaturation step of 30 s at 94° C., followed by 60 cycles of 5 s at 94° C., 5 s at 56° C., and 25 s at 72° C. A fluorescence lecture was taken after each elongation step (FIG. 9A). A standard curve for the PCR reaction was constructed by plotting cycle threshold values against log concentrations of each hMPV plasmid dilution. The number of hMPV copies present in clinical samples was calculated by interpolating cycle threshold values into the assay's standard curve (FIG. 9B).

### EXAMPLE VI

### Determination of hMPV Genotypes Based on Amplification and Sequencing of the Viral F and G Genes

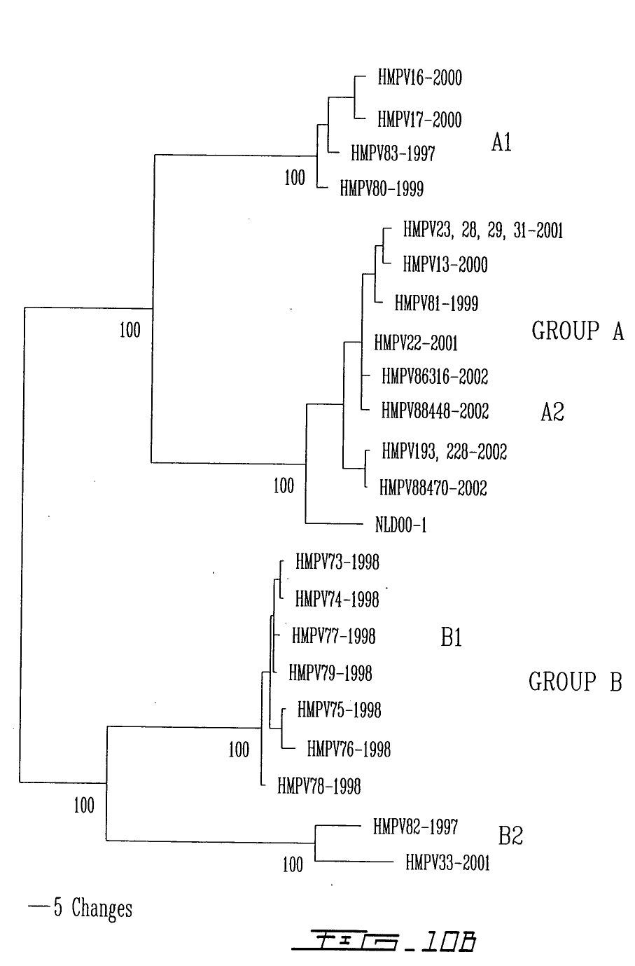

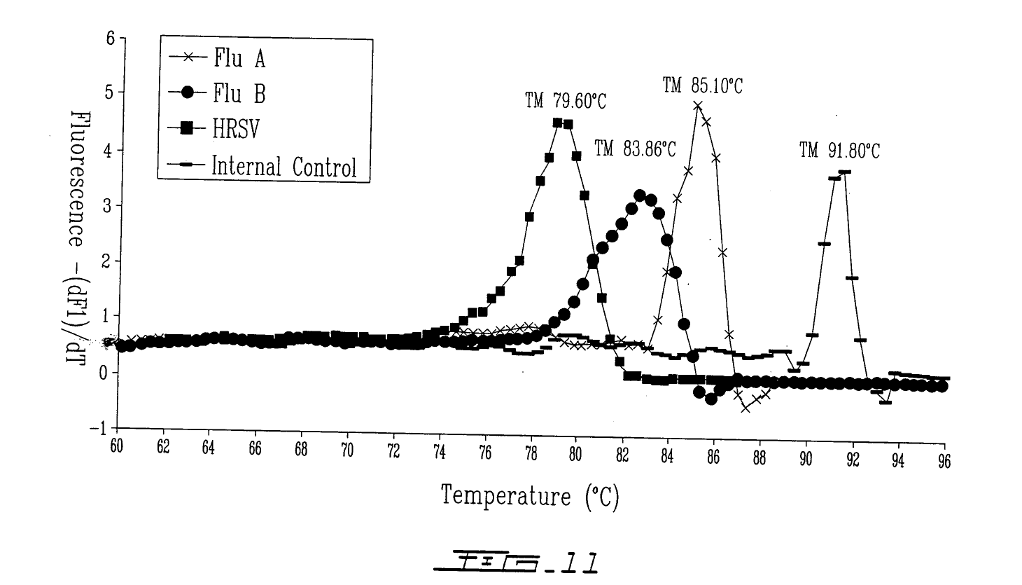

Determination of hMPV genotypes was achieved by PCR amplification of the F or G genes followed by DNA sequencing of the viral amplified products. Different sets of primers were used for PCR amplification and DNA sequencing of the F (SEQ. ID. Nos. 97 and 98) and G (SEQ. ID. Nos. 104 and 107 or 108 and 109) genes. Alignment of viral DNA sequences was done with the CLUSTAL W software version 1.7 for Unix and phylogenetic trees were computed by maximum parsimony, distance and maximum likelihood-based criteria analysis using PAUP version 4.0.d8. Phylogenetic trees for both the F (FIG. 10A) and G (FIG. 10B) genes from Canadian and non-Canadian viral isolates revealed the existence of two hMPV groups (genotypes A and B) with two subgroups (subtypes 1 and 2) within each group.

### EXAMPLE VII

### Comparison of Viral Culture and Multiplex PCR Assay for Detection of Influenza A Virus

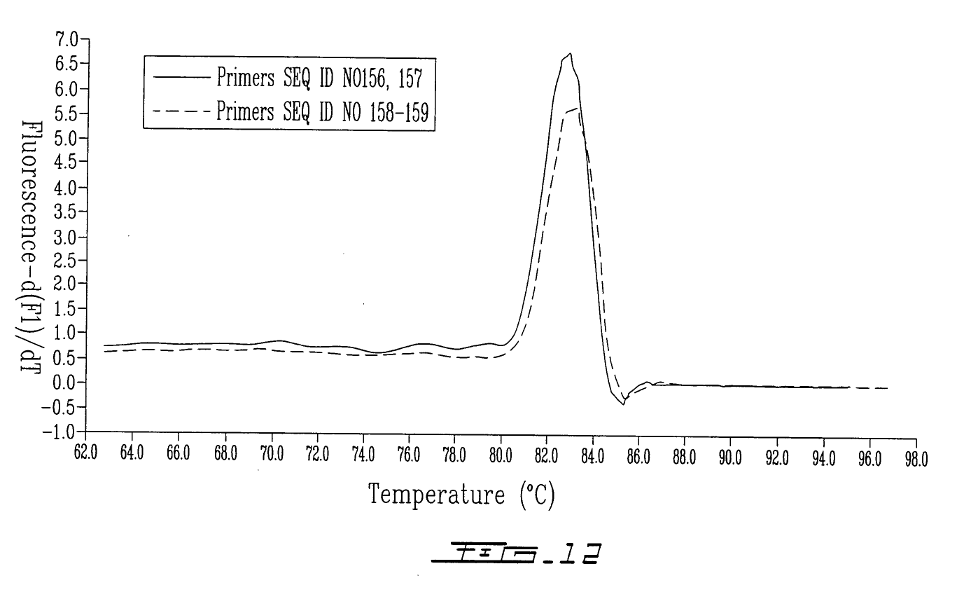

A multiplex PCR assay (including PCR primers defined in SEQ. ID. Nos. 139, 140, 141, 142, 143, and 144) aimed at detecting influenza A and B viruses and the respiratory syncytial virus was used to assess the etiology of severe acute respiratory tract infections (ARTI) necessitating hospitalization in children aged 0-3 years during the winter season 2001-02 at the CHUL, an university-based hospital, in Québec City (Canada). Nasopharyngeal (NPA) samples from all 208 children with ARTI were tested by multiplex PCR whereas a subset of 100 NPA specimens was inoculated on Madin Darby Canine Kidney (MDCK) cells for influenza culture. Briefly, viral RNA was prealably extracted from 200 μl of naso-pharyngeal aspirates using the QIAamp Viral RNA Mini kit (QIAGEN, Mississauga, ON, Canada). cDNA was then synthesized using 10 μl of the RNA preparation, 0.75 μM of random hexamer primers (Amersham Pharmacia Biotech, Baie d'Urfé, QC, Canada), 300 copies of an internal control template, and the Omniscript Reverse Transcriptase Kit (QIAGEN, Mississauga, ON, Canada) following the manufacturer's instructions. The internal control template consisted of a 558-bp transcribed region of an herpes simplex virus type 2 DNA polymerase region flanked by influenza B complementary primer sequences cloned into the pDrive plasmid (QIAGEN). cDNA was amplified using a real-time PCR procedure with the LC Faststart DNA Master SYBR Green 1 Kit (Roche Diagnostics, Laval, QC, Canada) in a LightCycler instrument (Roche Diagnostics). Each reaction had a total volume of 20 μl including 2 μl of complementary DNA and 18 μl of a reaction mixture containing 4 mM MgCl₂, 2 μl of LC Faststart DNA SYBR Green Master 1 Mix, 3% DMSO, 0.3 μM of influenza A and influenza B primers, and 0.8 μM of human respiratory syncytial virus primers (Table 10). Cycling conditions included an initial denaturation step of 10 min. at 94° C., followed by 50 cycles of 15 s at 94° C., 5 s at 58° C., and 25 s at 72° C. Detection of viral amplicons was characterized using the melting curve analysis feature of a real-time PCR instrument. Briefly, following the last amplification cycle on a LightCycler, the reaction temperature was rapidly increased to 94° C. and was then decreased to 60° C. for 30 seconds followed by a slow increase to 94° C. at a rate of 0.1° C. per second, with continuous fluorescence lecture. Using this procedure, the amplified products of influenza A, influenza B, hRSV, and the internal control could be differentiated by virtue of their specific Tm values i.e. 85, 83, 79, and 91° C., respectively (Table 13 and FIG. 11).

Influenza A virus was detected by multiplex PCR (specific Tm value of 85° C.) in 45/208 (21.6%) of the samples tested whereas the culture was positive for this virus in 14/100 (14%) tested NPA samples from children. For the subset of 100 NPA specimens tested by both methods, 88 (88%) had concordant results (11 positive, 77 negative) whereas discordant results were obtained for 12 (12%) samples (3 culture⁺/PCR⁻, 9 culture⁻/PCR⁺). Assuming that all positive results (by either culture or PCR) were true positive, the sensitivity values of the culture and the multiplex PCR for influenza A virus were 60.9% and 87.0%, respectively. There was no positive result for influenza B virus by any detection method as part of this study.

### EXAMPLE VIII

### Comparison of Antigen Detection and Multiplex PCR Assay for Detection of the Human Respiratory Syncytial Virus

It was realized as in Example 1, except that the multiplex PCR assay was used to detect hRSV from NPA samples obtained from 211 children with ARTI and compared with an antigen detection test for hRSV (TestPack, Abbott Laboratories, Abbott Park, Ill.).
hRSV was detected by the multiplex PCR (specific Tm value of 79° C.) in 109/211 (51.7%) of the tested samples whereas the hRSV antigen detection test was positive in 93/211 (44.1%) tested NPA samples from children. Concordant results were found in 183 (86.7%) samples (87 positive, 96 negative) whereas discordant results were obtained for 28 (13.3%) samples (6 antigen⁺/PCR⁻, 22 antigen⁻/PCR⁺). A second RT-PCR assay for detection of another hRSV gene (glycoprotein G) was used to resolve the discrepancies (Peret et al., 1998, J Gen Virol. 79:2221-2229). After the resolution of the discrepant results, the sensitivity values of the antigen detection test and the multiplex PCR for hRSV were 80.5% and 96.5%, respectively.

### EXAMPLE IX

### Real-Time Multiplex PCR Assay for Influenza Viruses and HRSV

Materials and Methods

Study population and antigenic test. Nasopharyngeal aspirates (NPA) were prospectively collected from children aged 0-3 years hospitalized for ARTI during the winter season 2001-02 at a single, university-based hospital, in Québec City (Canada) (1). The study was approved by the institutional review board of the Centre Hospitalier Universitaire de Québec. All samples were tested by the multiplex real-time PCR assay whereas antigen detection tests were performed on a subset of samples upon request by the treating physician. An aliquot (750 μl) of fresh NPA was first tested for the presence of HRSV and influenza A/B antigens using respectively the RSV TestPack (Abbott Laboratories, Abbott Park, Ill.) and the Directigen Flu A+B test (Becton Dickinson, Sparks, Md.). The rest of the specimen was frozen at −80° C. for a maximum of 3 months before nucleic acid extraction and PCR testing.

RNA extraction and cDNA synthesis. Viral RNA was extracted from 200 μl of NPA samples using the QIAamp Viral RNA Mini kit (QIAGEN, Missisauga, ON, Canada). Complementary DNA was then synthesized using 10 μl of the RNA preparation, 0.75 μM of random hexamer primers (Amersham Pharmacia Biotech, Baie d'Urfé, QC, Canada), and the Omniscript Reverse Transcriptase kit (QIAGEN) in presence of 300 copies of an internal control. The internal control template consisted of a 558-bp transcribed region of the herpes simplex virus type 2 DNA polymerase gene flanked by influenza B complementary primer sequences (see below) cloned into the pDrive plasmid (QIAGEN).

Real-time multiplex and conventional PCR assays. The multiplex PCR respiratory assay was designed to amplify conserved regions of the influenza A (7) and B (14) matrix genes as well as the fusion gene of HRSV (17). Complementary DNA was amplified using a real-time PCR procedure with the LC Faststart DNA Master SYBR Green 1 kit (Roche Diagnostics, Laval, QC, Canada) in a LightCycler instrument (Roche Diagnostics). Each reaction had a total volume of 20 μl including 2 μl of cDNA and 18 μl of a reaction mixture containing 2.4 mM MgCl₂, 2 μl of Faststart DNA SYBR Green 1 Master Mix, 3% DMSO, 0.3 mM of influenza A and B primers (SEQ. ID Nos. 139-140 and 141-142), and 0.8 mM of HRSV primers (SEQ. ID Nos. 143-144). Cycling conditions included an initial denaturation step of 10 min at 94° C., followed by 50 cycles of 15 s at 94° C., 5 s at 58° C., and 25 s at 72° C. At the end of each cycle, the fluorescent signal was measured at a wavelength of 530 nM using the LightCycler Fluorimeter. The melting curve analysis program of the LightCycler was used to identify specific PCR products. Briefly, following the last amplification cycle, the reaction temperature was rapidly increased to 94° C. and was then decreased to 60° C. for 30 s followed by a slow increase to 94° C. at a rate of 0.1° C. per s, with continuous fluorescence monitoring. For resolution of discrepant results between the multiplex PCR assay and antigenic tests, conventional RT-PCR assays for detection of the glycoprotein G gene of HRSV (22) and the M2 gene of influenza A viruses (13) were performed retrospectively.

Results

Assay's characteristics. The sensitivity of each individual real-time PCR assay and of the multiplex PCR respiratory assay was determined for each target by testing serial dilutions of transcribed plasmids containing specific viral sequences. The lower limit of detection for influenza A, influenza B, and HRSV was found to be 10, 50 and 50 copies, respectively, in individual PCR assays whereas it was 50 copies for each target (10 out of 10 times for influenza A and 9 out of 10 times for influenza B and HRSV) in the multiplex PCR test. No amplification signal was detected in the real-time multiplex assay when testing viral DNA or RNA from adenoviruses, rhinoviruses, enteroviruses (echo 11), parainfluenza viruses 1-3, human metapneumovirus, and herpesviruses (herpes simplex virus type 1 and 2, cytomegalovirus, and varicella-zoster virus). Specific melting temperature (Tm) values were first determined by testing a set of clinical isolates and vaccine strains (11 influenza A/H1, 10 influenza A/H3, 10 influenza B, and 17 HRSV) from different years (Table 14). A representative set of results is shown in FIG. 11. The mean Tm values were 85.27±0.22, 83.47±0.46, and 79.51±0.30° C. for influenza A, influenza B, and HRSV corresponding to fragments of 245, 524, and 380 bp, respectively, after gel electrophoresis (Table 14). We found no influence of influenza A subtype (H1 and H3) and HRSV genotype (A and B) on the Tm values (Table 14). The addition of 300 copies of the internal control in the assay's master mix resulted in a specific amplification peak with a Tm value of 91° C. (558 bp) in the absence of amplified viral products.

Evaluation of the multiplex PCR respiratory assay in a pediatric population. The multiplex PCR assay was evaluated using extracted RNA from 208 NPA samples collected during a prospective pediatric study aimed at assessing the role of the human metapneumovirus in hospitalized children (0-3 years) with ARTI (1). The number of positive samples by multiplex PCR for influenza A, influenza B, and HRSV was found to be 45 (21.6%), 0 (0%), and 106 (51.0%), respectively. Overall, the rate of positivity for any of the 3 viruses was 66.8% (139/208) including 5.8% (12/208) of co-infections with influenza A and HRSV. None of the samples was considered to contain PCR-inhibitory samples as verified by the amplification of the internal control in all PCR-negative samples. The ranges of Tm values for influenza A- and HRSV-positive samples were 85.10±0.39 and 79.60±0.42° C., respectively. All amplified products with one of the latter Tm values were confirmed as specific viral targets by visualizing the appropriate band on agarose gel. As none of the samples contained influenza B virus sequences (which correlates with the very occasional isolation of influenza B viruses in the Québec City area during the 2001-2002 season), a clinical evaluation of the multiplex PCR assay was conducted for influenza A and HRSV only.

Evaluation of the multiplex PCR respiratory assay for detecting influenza A virus. From the 208 NPA evaluated by multiplex PCR, a subset of 172 samples were also tested for the presence of influenza antigens using the Directigen Flu A+B test (Table 15). Concordant results were obtained for 145 (84.3%) samples including 17 positive and 128 negative results by the two tests. A total of 27 (15.7%) samples showed discordant results with 25 being positive by the multiplex PCR assay only and two positive by the antigenic test only. In order to resolve the discrepant results, a second RT-PCR test targeting a conserved gene (M2) of influenza A viruses was performed as reported (13). As shown in Table 15, influenza A virus M2 sequences were detected by the conventional RT-PCR assay in 22 (81.5%) of the 27 discordant samples. Using the M2 RT-PCR assay as the reference test, the sensitivity, specificity, positive predictive and negative predictive values of the multiplex real-time PCR assay and the antigenic test for influenza A virus were 100%, 97.7%, 92.8%, 100% and 43.6%, 98.5%, 89.5%, 85.6%, respectively. The multiplex PCR respiratory assay was positive for HRSV in 12 (44.4%) of the 27 discordant influenza A samples with 10 of these samples showing dual HRSV/influenza A infections by the multiplex PCR assay (Table 16). The mean cycle threshold (C_{T}) values, which are inversely correlated with the amounts of viral RNA, were 28.1 and 33.0 for PCR-positive/antigen-positive and PCR-positive/antigen-negative samples, respectively (Table 16). These values correspond to approximately 5000 and 50 copies, respectively, when a specific influenza A plasmid was tested in the multiplex PCR assay.

Evaluation of the multiplex PCR respiratory assay for detecting HRSV. A total of 204 NPA samples could be evaluated by both the multiplex real-time PCR assay and the presence of HRSV antigens using the RSV TestPack (Table 17). Concordant results were found in 172 (84.3%) samples including 83 positive and 89 negative results by the two tests. Thirty-two (15.7%) samples had discordant results with 21 being positive by the multiplex PCR assay only and 11 by the antigenic test only. Conventional RT-PCR tests for the HRSV glycoprotein G (A and B subtypes) were performed as previously described (22) to resolve discrepant results. Specific sequences were detected by RT-PCR assays for the HRSV glycoprotein G gene in 26 (81.2%) of the 32 discordant samples including 11 A and 15 B strains (Table 18). Using the latter test as “gold standard”, the sensitivity, specificity, positive predictive and negative predictive values of the multiplex real-time PCR assay and the antigenic test for HRSV were 94.5%, 98.9%, 99.0%, 94.0% and 81.6%, 94.7%, 94.7%, 81.8%, respectively. Four (66.7%) of the six false-negative HRSV results by multiplex PCR were positive for influenza A (Table 18). The mean CT values of the PCR-positive/antigen-positive and PCR-positive/antigen-negative samples were 28.6 and 32.7, respectively (Table 18). These values correspond to approximately 1000 and 100 copies, respectively, when a specific HRSV plasmid was tested in the multiplex PCR assay.

Discussion

In this example, a new multiplex real-time PCR assay for the most commonly detected respiratory viral pathogens, namely influenza viruses (A and B) and HRSV is described. The key features of this novel assay include its rapidity (turnaround time of approximately 2h30 including 30 min for sample preparation, 60 min for RT and 60 min for real-time PCR including viral identification), and its sensitivity (50 copies per assay for each viral target). Our results clearly demonstrate that our multiplex real-time PCR assay is more sensitive than commercially-available antigenic detection tests for both influenza A and HRSV with the greatest difference in sensitivity noted for influenza viruses.

Multiplex RT-PCR assays targeting up to nine different respiratory pathogens have been previously reported (6, 8, 12, 21, 26, 29). More recently, a TaqMan-based real-time PCR assay for simultaneous detection of influenza A and B viruses has also been described (31). Compared to conventional multiplex PCR assays, our real-time PCR test is much more rapid because it avoids additional nested amplification and/or hybridization steps required for identification of viral products. Furthermore, the real-time amplification procedure minimizes the chances of contamination because there is no post-PCR processing of the samples. Although our multiplex real-time PCR assay based on melting curve analysis of amplicons does not permit absolute quantification of the viral targets as in the TaqMan PCR procedure (31), it allows for a larger number of viral targets to be simultaneously detected since there is no limitation related to the capability of the system to detect multiple dyes linked to detection probes with distinct emission wavelengths. Eventually, more than three viral targets could be simultaneously detected in the LightCycler assay assuming the absence of interaction between PCR primers and a reproducible and discriminative Tm value for each viral amplicon.

Evaluation of our multiplex real-time PCR assay in a pediatric population with severe ARTI clearly illustrated its clinical potential. Indeed, our assay identified a viral pathogen (influenza or HRSV) in two-thirds of the hospitalized children. These numbers are similar to the ones previously reported by our group in the same population using individual real-time assays for these viral pathogens (1). Notably, it was possible to show that 6% of hospitalized children had dual viral infections, a finding that has been reported albeit at a lower frequency by other investigators (8, 12, 21). Rapid identification of theses viral pathogens (influenza and HRSV) is of paramount importance for isolation purposes in the hospital setting and early institution of specific antiviral therapy (32). We are currently designing additional multiplex assays based on the same technology to cover the whole spectrum of respiratory pathogens including parainfluenza viruses (PIV), adenoviruses, enteroviruses, and coronaviruses (27). To this list, the recently described human metapneumovirus which has been reported by our group can be added and others in up to 10% of ARTI in hospitalized children (1, 11, 20, 30).

A specific evaluation of the real-time multiplex PCR assay for influenza A revealed that it was twice as sensitive as the rapid antigen detection test in use at our institution during the winter season of 2001-2002. None of the two PCR-negative, antigen-positive samples were confirmed by a second PCR targeting another conserved gene (M2) of influenza A whereas most (22/25, 88.0%) multiplex PCR-positive, antigen-negative were. In general, the false-negative antigenic test results could be explained by low amounts of viral RNA in those NPA samples as demonstrated by high C_{T} values in the corresponding real-time PCR assays. Three multiplex PCR test results were not confirmed by the influenza A M2 PCR test and were thus considered false-positive results. However, as we have not formally evaluated the sensitivity of the M2 PCR test and since viral cultures were not routinely done, we cannot rule out the possibility that such discrepant results were indeed true positive. The superiority of RT-PCR tests has been shown over conventional methods (antigenic tests and viral culture) for detecting influenza viruses in clinical samples including those sent to the laboratory by mail (2, 4, 12, 25, 31). It is now common procedure to confirm any negative antigenic tests results for influenza A and B by either viral culture or RT-PCR considering the high specificity but relatively poor sensitivity of the former assays.

For HRSV, the multiplex real-time PCR assay was also found to be more sensitive (by approximately 15%) than the rapid antigenic test when a second RT-PCR assay for the HRSV gG gene was used to resolve the discrepancies. Most of the false-negative antigenic test results could be explained by low amounts of viral RNA in the NPA samples as shown by mean C_{T} values of 32.7 for discordant results compared to 28.6 for concordant positive results. The specificity of the multiplex assay was excellent with only one positive result not confirmed by the second PCR test. In contrast, the multiplex PCR assay missed six cases of HRSV which tested positive in both the antigenic test and the second PCR assay for HRSV gG. Notably, four of those six samples tested positive for influenza A in the multiplex PCR assay. Thus, although the multiplex PCR test has the capability of detecting dual infections (as seen in 6% of our cases), it is possible that high amounts of one virus could inhibit amplification of other pathogens. It is noteworthy that the multiplex PCR assay had the ability to amplify both HRSV genotypes as shown by the detection of 15 B and 11 A genotypes using specific PCR assays aimed at detecting variable regions of the HRSV gG gene (17). Although the rapid antigenic test had a relatively good performance (sensitivity: 82%, specificity 95%) for detection of HRSV in young children, the real advantage of PCR probably resides in the adult population where viral titers are lower and where antigenic tests are not recommended with upper respiratory tract samples (5).

The absence of circulation of influenza B viruses during the study period prevents adequate validation of our multiplex assay for this pathogen and in particular for its ability to detect mixed (influenza B and HRSV) infections. Also, the performance of our multiplex PCR assay cannot be generalized to other types of samples (throat swabs, sputum) and to other populations (outpatients, adults) at the present time. Also, the absence of serological testing in the present study may have underestimated the rate of viral infections especially for those children who presented late after the onset of symptoms. Finally, although it was relatively easy to discriminate between the different viral pathogens based on their specific Tm values only (Table 14), we sometimes had to confirm viral amplicons by gel electrophoresis, which increases the assay's turnaround time.

This new assay is as specific and much more sensitive than currently-available antigen detection tests and could complement the latter in the hospital setting when there is high clinical suspicion despite negative results.

### CITED REFERENCES IN EXAMPLE IX

1. Boivin, G., G. De Serres, S. Côté, R. Gilca, Y. Abed, L. Rochette, M. G. Bergeron, and P. Déry. 2003. Human metapneumovirus infections in hospitalized children. Emerg. Infect. Dis. 9:634-640.
2. Boivin, G., I. Hardy, and A. Kress. 2001. Evaluation of a rapid optical immunoassay for influenza viruses (FLU OIA Test) in comparison with cell culture and reverse transcription-PCR. J. Clin. Microbiol. 39:730-732.
3. Dowell, S. F., L. J. Anderson, H. E. Gary, Jr., D. D. Erdman, Jr. F. Plouffe, T. M. File, Jr., B. J. Marston, and R. F. Breiman. 1996. Respiratory syncytial virus is an important cause of community-acquired lower respiratory infection among hospitalized adults. J. Infect. Dis. 174:456-462.
4. Ellis, J. S., D. M. Fleming, and M. C. Zambon. 1997. Multiplex reverse transcription-PCR for surveillance of influenza A and B viruses in England and Wales in 1995 and 1996. J. Clin. Microbiol. 35:2076-2082.
5. Englund, J. A., P. A. Piedra, A. Jewell, K. Patel, B. B. Baxter, and E. Whimbey. 1996. Rapid diagnosis of respiratory syncytial virus infections in immunocompromised adults. J. Clin. Microbiol. 34:1649-1653.
6. Fan, J., K. J. Henrickson, and L. L. Savatski. 1998. Rapid simultaneous diagnosis of infections with respiratory syncytial viruses A and B, influenza viruses A and B, and human parainfluenza virus types 1, 2, and 3 by multiplex quantitative reverse transcription-polymerase chain reaction-enzyme hybridization assay (Hexaplex). Clin. Infect. Dis. 26:1397-1402.
7. Fouchier, R. A., T. M. Bestebroer, S. Herfst, L. Van Der Kemp, G. F. Rimmelzwaan, and A. D. Osterhaus. 2000. Detection of influenza A viruses from different species by PCR amplification of conserved sequences in the matrix gene. J. Clin. Microbiol. 38:4096-4101.
8. Grondahl, B., W. Puppe, A. Hoppe, I. Kuhne, J. A. Weigl, and H. J. Schmitt. 1999. Rapid identification of nine microorganisms causing acute respiratory tract infections by single-tube multiplex reverse transcription-PCR: feasibility study. J. Clin. Microbiol. 37:1-7.
9. Han, L. L., J. P. Alexander, and L. J. Anderson. 1999. Respiratory syncytial virus pneumonia among the elderly: a assessment of disease burden. J. Infect. Dis. 179:25-30.
10. Hayden, F. G., A. D. Osterhaus, J. J. Treanor, D. M. Fleming, F. Y. Aoki, K. G. Nicholson, A. M. Bohnen, H. M. Hirst, O. Keene, and K. Wightman. 1997. Efficacy and safety of the neuraminidase inhibitor zanamivir in the treatment of influenzavirus infections. GG167 Influenza Study Group. N. Engl. J. Med. 337:874-880.
11. Jartti, T., B. van den Hoogen, R. P. Garofalo, A. D. Osterhaus, and O. Ruuskanen. 2002. Metapneumovirus and acute wheezing in children. Lancet. 360:1393-1394.
12. Kehl, S. C., K. J. Henrickson, W. Hua, and J. Fan. 2001. Evaluation of the Hexaplex assay for detection of respiratory viruses in children. J. Clin. Microbiol. 39:1696-1701.
13. Klimov, A. I., E. Rocha, F. G. Hayden, P. A. Shult, L. F. Roumillat, and N. J. Cox. 1995. Prolonged shedding of amantadine-resistant influenza A viruses by immunodeficient patients: detection by polymerase chain reaction-restriction analysis. J. Infect. Dis. 172:1352-1355.
14. Li, J., S. Chen, and D. H. Evans. 2001. Typing and subtyping influenza virus using DNA microarrays and multiplex reverse transcriptase PCR. J. Clin. Microbiol. 39:696-704.
15. Liolios, L., A. Jenney, D. Spelman, T. Kotsimbos, M. Catton, and S. Wesselingh. 2001. Comparison of a multiplex reverse transcription-PCR-enzyme hybridization assay with conventional viral culture and immunofluorescence techniques for the detection of seven viral respiratory pathogens. J. Clin. Microbiol. 39:2779-2783.
16. Makela, M. J., K. Pauksens, T. Rostila, D. M. Fleming, C. Y. Man, O. N. Keene, and A. Webster. 2000. Clinical Efficacy and Safety of the Orally Inhaled Neuraminidase Inhibitor Zanamivir in the Treatment of Influenza: a Randomized, Double-blind, Placebo-controlled European Study. J. Infect. 40:42-48.
17. Mazzulli, T., T. C. Peret, A. McGeer, D. Cann, K. S. MacDonald, R. Chua, D. D. Erdman, and L. J. Anderson. 1999. Molecular characterization of a nosocomial outbreak of human respiratory syncytial virus on an adult leukemia/lymphoma ward. J. Infect. Dis. 180:1686-1689.
18. Mist Study Group. 1998. Randomised trial of efficacy and safety of inhaled zanamivir in treatment of influenza A and B virus infections. Lancet. 352:1877-1881.
19. Neuzil, K. M., B. G. Mellen, P. F. Wright, E. F. Mitchel, and M. R. Griffin. 2000. The effect of influenza on hospitalizations, outpatient visits, and courses of antibiotics in children. N. Engl. J. Med. 342:225-231.
20. Nissen, M. D., D. J. Siebert, I. M. Mackay, T. P. Sloots, and S. J. Withers. 2002. Evidence of human metapneumovirus in Australian children. Med. J. Aust. 176:188.
21. Osiowy, C. 1998. Direct detection of respiratory syncytial virus, parainfluenza virus, and adenovirus in clinical respiratory specimens by a multiplex reverse transcription-PCR assay. J. Clin. Microbiol. 36:3149-3154.
22. Peret, T. C., C. B. Hall, K. C. Schnabel, J. A. Golub, and L. J. Anderson. 1998. Circulation patterns of genetically distinct group A and B strains of human respiratory syncytial virus in a community. J. Gen. Virol. 79:2221-2229.
23. Simoes, E. A. 1999. Respiratory syncytial virus infection. Lancet. 354:847-852.
24. Simonsen, L., K. Fukuda, L. B. Schonberger, and N. J. Cox. 2000. The impact of influenza epidemics on hospitalizations. J. Infect. Dis. 181:831-837.
25. Steininger, C., M. Kundi, S. W. Aberle, J. H. Aberle, and T. Popow-Kraupp. 2002. Effectiveness of reverse transcription-PCR, virus isolation, and enzyme-linked immunosorbent assay for diagnosis of influenza A virus infection in different age groups. J. Clin. Microbiol. 40:2051-2056.
26. Stockton, J., J. S. Ellis, M. Saville, J. P. Clewley, and M. C. Zambon. 1998. Multiplex PCR for typing and subtyping influenza and respiratory syncytial viruses. J. Clin. Microbiol. 36:2990-2995.
27. Treanor, J. 2002. Respiratory Infections, p. 7-26, In Richard. J. Whitley, Douglas D. Richman and Frederick G. Hayden (eds.). Clinical Virology. 2^{nd} ed. ASM Press. Washington, D.C.
28. Treanor, J. J., F. G. Hayden, P. S. Vrooman, R. Barbarash, R. Bettis, D. Riff, S. Singh, N. Kinnersley, P. Ward, and R. G. Mills. 2000. Efficacy and safety of the oral neuraminidase inhibitor oseltamivir in treating acute influenza: a randomized controlled trial. US Oral Neuraminidase Study Group. JAMA. 283:1016-1024.
29. Valassina, M., A. M. Cippone, M. G. Cusi, and P. E. Valensin. 1996. Rapid detection of different RNA respiratory virus species by multiplex RT-PCR: application to clinical specimens. Clin. Diag. Virol. 8:227-232.
30. van den Hoogen, B. G., J. C. de Jong, J. Groen, T. Kuiken, R. de Groot, R. A. Fouchier, and A. D. Osterhaus. 2001. A newly discovered human pneumovirus isolated from young children with respiratory tract disease. Nat. Med. 7:719-724.
31. van Elden, L. J., M. Nijhuis, P. Schipper, R. Schuurman, and A. M. van Loon. 2001. Simultaneous detection of influenza viruses A and B using real-time quantitative PCR. J. Clin. Microbiol. 39:196-200.
32. Whitley, R. J., F. G. Hayden, K. S. Reisinger, N. Young, R. Dutkowski, D. Ipe, R. G. Mills, and P. Ward. 2001. Oral oseltamivir treatment of influenza in children. Pediatr. Infect. Dis. J. 20:127-133.

### EXAMPLE X

### Description and Evaluation of the Multiplex Respiratory-1 PCR

Amplification was performed as in Example 9, except that hMPV primers (as defined in SEQ. ID. Nos. 95 and 96 or 112 and 114) were added to the multiplex PCR assay (already containing primers of SEQ. ID. Nos. 139+140 for influenza A, 141+142 for influenza B, and 143+144 for hRSV) at a concentration of 0.3 μM. The cycling conditions included an amplification step of 50 cycles of 15 s at 94° C., 5 s at 58° C., and 28 s at 72° C. The Tm values determined with the LightCycler instrument were 91° C., 85° C., 83° C., 79° C., and 82° C./77° C. for the internal control, influenza A, influenza B, hRSV, and hMPV N/L genes, respectively (FIG. 7A for hMPV N primers of SEQ. ID. Nos. 95 and 96 and FIG. 7B for hMPV L primers of SEQ. ID Nos. 112 and 114). The lower limit of detection of the multiplex respiratory-1 PCR was 10, 50, 100, and 100 copies for influenza A, influenza B, hRSV, and hMPV, respectively, in the presence of 500 copies of an internal control (plasmid flanked by influenza B complementary primer sequences). The multiplex respiratory-1 assay was found to be specific with no amplification of adenoviruses, enteroviruses, rhinoviruses, parainfluenza viruses 1-3, coronaviruses, herpes simplex virus type 1 and 2, varicella-zoster virus, cytomegalovirus, HHV-6, HHV-8, and Epstein-Barr virus.

The multiplex respiratory-1 PCR was evaluated with NPA samples from 248 children ≦3 years with acute respiratory tract infections and 108 adults ≧50 years with exacerbation of chronic obstructive pulmonary disease or pneumonia requiring hospitalization during the 2002-03 winter/spring period in Quebec City, Canada. In children, the detection rate for influenza A, influenza B, hRSV, and hMPV was 9/248 (3.6%), 4/248 (1.6%), 123/248 (49.6%), and 13/248 (5.2%), respectively. Five (2.0%) of the 248 children had a mixed viral infection. None of the NPA samples was found to contain PCR-inhibitory substances as shown by successful amplification of the internal control in all negative samples. In adults, the detection rate for influenza A, influenza B, hRSV, and hMPV was 3/108 (2.8%), 0/108 (0%), 13/108 (12.0%), and 2/108 (1.8%), respectively. One patient had a mixed viral infection (influenza A and hMPV). No samples contained PCR-inhibitory samples.

### EXAMPLE XI

### Description and Evaluation of the Multiplex Respiratory-2 PCR

Same as example 9, except that the multiplex PCR assay is composed of the following PCR primers for detection of parainfluenza-1 (SEQ. ID. Nos. 147 and 148), parainfluenza-3 (SEQ. ID. Nos. 151 and 152), human adenoviruses (SEQ. ID. Nos. 153 and 155), and rhinoviruses/enteroviruses (SEQ. ID. Nos. 160 and 161). Primers are added in the PCR reaction mixture at a concentration of 0.2 μM (SEQ. ID. Nos. 147 and 151), 0.3 μM (SEQ. ID. Nos. 153 and 155), 0.4 μM (SEQ. ID. Nos. 148 and 152), and 0.5 μM (SEQ. ID. Nos. 160 and 161). The cycling conditions included an amplification step of 50 cycles of 15 s at 94° C., 5 s at 58° C., and 26 s at 72° C. The Tm values determined with the LightCycler instrument were 91.0° C., 80.0° C., 77.5° C., 89.0° C., 82.0° C., and 85.0° C. for the internal control, parainfluenza-1, parainfluenza-3, adenoviruses, rhinoviruses, and enteroviruses, respectively (FIG. 8). The lower limit of detection of the multiplex respiratory-2 PCR was 50, 100, 10, 50, and 200 copies for parainfluenza-1, parainfluenza-3, adenoviruses, rhinoviruses, and enteroviruses, respectively, in the presence of 500 copies of an internal control (plasmid flanked by rhinovirus/enterovirus complementary primer sequences). The multiplex respiratory-2 assay was found to be specific with no amplification of influenza A, influenza B, hRSV, hMPV, coronavirus, herpes simplex virus type 1 and 2, varicella-zoster virus, cytomegalovirus, HHV-6, HHV-8, and Epstein-Barr virus.

### EXAMPLE XII

### Description of Real-Time PCR Assays for the SARS-Coronavirus

Amplification has been performed as in Example 9, except that the PCR assay is composed uniquely of primers (two different possible sets) for the SARS-coronavirus as defined in SEQ. ID. Nos. 156-157 and 158-159. The concentration of the primers in the PCR reaction mixture is 0.5 μM. The amplification step included 50 cycles of 15 s at 94° C., 5 s at 58° C., and 20 s at 72° C. The Tm for both PCR products is 83° C. (FIG. 12).

TABLE 1
List of hMPV sequences revealed in the present invention

| SEQ ID NO. | Isolate No. | Target gene |
|---|---|---|
| 2 | hMPV-118 | Nucleoprotein |
| 3 | hMPV-174 | Nucleoprotein |
| 4 | hMPV-176 | Nucleoprotein |
| 5 | hMPV-193 | Nucleoprotein |
| 6 | C-77807 | Nucleoprotein |
| 7 | C-77748 | Nucleoprotein |
| 8 | hMPV-217 | Nucleoprotein |
| 10 | hMPV-163 | Nucleoprotein |
| 11 | hMPV-174 | Nucleoprotein |
| 12 | hMPV-184 | Nucleoprotein |
| 13 | hMPV-185 | Nucleoprotein |
| 14 | C-76837 | Nucleoprotein |
| 15 | C-77807 | Nucleoprotein |
| 16 | C-78123 | Nucleoprotein |
| 17 | C-78132 | Nucleoprotein |
| 18 | C-78163 | Nucleoprotein |
| 19 | C-91205 | Nucleoprotein |
| 20 | C-91364 | Nucleoprotein |
| 21 | C-91830 | Nucleoprotein |
| 22 | hMPV-Q86 | Nucleoprotein |
| 23 | hMPV-Q97 | Nucleoprotein |
| 24 | Can 97-83 | Nucleoprotein |
| 25 | Can 98-75 | Nucleoprotein |
| 26 | Can 98-75 | Fusion type II |
| 27 | C-67345 | Fusion type I |
| 28 | Can 97-83 | Fusion type I |
| 29 | C-68661 | Fusion type I |
| 30 | C-73750 | Fusion type I |
| 31 | C-74549 | Fusion type I |
| 32 | C-74783 | Fusion type I |
| 33 | C-76837 | Fusion type I |
| 34 | C-77748 | Fusion type I |
| 35 | C-77807 | Fusion type I |
| 36 | C-78132 | Fusion type I |
| 37 | C-78163 | Fusion type I |
| 38 | C-78406 | Fusion type I |
| 39 | hMPV-174 | Fusion type II |
| 40 | hMPV-193 | Fusion type I |
| 41 | hMPV-194 | Fusion type I |
| 42 | hMPV-198 | Fusion type I |
| 43 | hMPV-217 | Fusion type II |
| 44 | hMPV-208 | Fusion type I |
| 45 | hMPV-223 | Fusion type I |
| 46 | hMPV-228 | Fusion type I |
| 47 | C-85885 | Fusion type I |
| 48 | C-60298 | Fusion type I |
| 49 | C-63778 | Fusion type II |
| 50 | C-64199 | Fusion type II |
| 51 | C-64200 | Fusion type II |
| 52 | C-64536 | Fusion type II |
| 53 | C-73769 | Fusion type II |
| 54 | C-74501 | Fusion type II |
| 55 | C-78429 | Fusion type II |
| 56 | C-78562 | Fusion type II |
| 57 | hMPV-118 | Fusion type II |
| 58 | hMPV-200 | Fusion type I |
| 59 | hMPV-176 | Fusion type I |
| 60 | hMPV-193 | Glycoprotein |
| 61 | hMPV-228 | Glycoprotein |
| 62 | C-77748 | Glycoprotein |
| 63 | C-78132 | Glycoprotein |
| 64 | C-78406 | Glycoprotein |
| 65 | C-73750 | Glycoprotein |
| 66 | C-60298 | Glycoprotein |
| 67 | C-74783 | Glycoprotein |
| 68 | C-74549 | Glycoprotein |
| 69 | C-77807 | Glycoprotein |
| 70 | C-78163 | Glycoprotein |
| 71 | C-68661 | Glycoprotein |
| 72 | C-88448 | Glycoprotein |
| 73 | C-88470 | Glycoprotein |
| 74 | C-86316 | Glycoprotein |
| 76 | hMPV-118 | Polymerase |
| 77 | hMPV-163 | Polymerase |
| 78 | hMPV-174 | Polymerase |
| 79 | hMPV-184 | Polymerase |
| 80 | hMPV-196 | Polymerase |
| 81 | C-76837 | Polymerase |
| 82 | C-77807 | Polymerase |
| 83 | hMPV-185 | Polymerase |
| 84 | C-78123 | Polymerase |
| 85 | C-91205 | Polymerase |
| 86 | C-91364 | Polymerase |
| 87 | C-91749 | Polymerase |
| 88 | hMPV-Q86 | Polymerase |
| 89 | hMPV-Q97 | Polymerase |
| 90 | Can 97-83 | Polymerase |

TABLE 2
List of hMPV PCR primers developed in the present invention

| | | Originating DNA | |
|---|---|---|---|
| SEQ ID NO. | Target gene | Position^{a} | SEQ ID NO. |
| 91 | Nucleoprotein (N) | 25 | 1 |
| 92 | Nucleoprotein (N) | 97 | 1 |
| 93 | Nucleoprotein (N) | 146^{b} | 1 |
| 94 | Nucleoprotein (N) | 1025^{b} | 1 |
| 95 | Nucleoprotein (N) | 58 | 9 |
| 96 | Nucleoprotein (N) | 986^{b} | 9 |
| 97 | Fusion (F) | 3052 | 1 |
| 98 | Fusion (F) | 3810^{b} | 1 |
| 99 | Fusion (F) | 4671^{b} | 1 |
| 100 | Matrix (M) | 2165 | 1 |
| 101 | Matrix (M) | 2943^{b} | 1 |
| 102 | Phosphoprotein (P) | 1248 | 1 |
| 103 | Phosphoprotein (P) | 2124^{b} | 1 |
| 104 | Glycoprotein (G) | 6231 | 1 |
| 105 | Glycoprotein (G) | 6247 | 1 |
| 106 | Glycoprotein (G) | 6957^{b} | 1 |
| 107 | Glycoprotein (G) | 6981^{b} | 1 |
| 108 | Glycoprotein (G) | 6235 | 1 |
| 109 | Glycoprotein (G) | 7227^{b} | 1 |
| 110 | RNA polymerase (L) | 142 | 75 |
| 111 | RNA polymerase (L) | 691^{b} | 75 |
| 112 | RNA polymerase (L) | 7308 | 1 |
| 113 | RNA polymerase (L) | 7857^{b} | 1 |
| 114 | RNA polymerase (L) | 7616 | 1 |

^{a}Position refers to nucleotide position of 5′ end of primer. Oligonucleotides are 17-20 bp in length.
^{b}Primer is reverse-complement of target sequence.

TABLE 3
List of hMPV adjacent probes developed in the present invention.

| | | Originating DNA | |
|---|---|---|---|
| SEQ ID NO. | Target gene | Position^{a} | SEQ ID NO. |
| 115 | Nucleoprotein (N) | 786 | 1 |
| 116 | Nucleoprotein (N) | 808 | 1 |
| 117 | Fusion (F) | 3163 | 1 |
| 118 | Fusion (F) | 3187 | 1 |
| 119 | Matrix (M) | 2636 | 1 |
| 120 | Matrix (M) | 2659 | 1 |
| 121 | Phosphoprotein (P) | 1492 | 1 |
| 122 | Phosphoprotein (P) | 1518 | 1 |
| 123 | Glycoprotein (G) | 6509 | 1 |
| 124 | Glycoprotein (G) | 6530 | 1 |
| 125 | RNA polymerase (L) | 7600 | 1 |
| 126 | RNA polymerase (L) | 7627 | 1 |
| 127 | RNA polymerase (L) | 7331 | 1 |
| 128 | Nucleoprotein (N) | 824 | 9 |
| 129 | RNA polymerase (L) | 434 | 75 |

^{a}Position refers to nucleotide position of 5′ end of probe. Oligonucleotides are 21-25 bp in length.

TABLE 4
Length of amplicons obtained with different hMPV primer
pairs which are objects of the present invention

| | Target | Length of |
|---|---|---|
| SEQ ID NO. | Gene | amplicon |
| 91 + 94 | Nucleoprotein (N) | 1001 bp |
| 92 + 94 | Nucleoprotein (N) | 929 bp |
| 91 + 93 | Nucleoprotein (N) | 122 bp |
| 97 + 99 | Fusion (F) | 1620 bp |
| 97 + 98 | Fusion (F) | 759 bp |
| 100 + 101 | Matrix (M) | 779 bp |
| 102 + 103 | Phosphoprotein (P) | 877 bp |
| 104 + 107 | Glycoprotein (G) | 751 bp |
| 105 + 106 | Glycoprotein (G) | 711 bp |
| 108 + 109 | Glycoprotein (G) | 993 bp |
| 112 + 113 | RNA polymerase (L) | 550 bp |
| 112 + 114 | RNA polymerase (L) | 308 bp |

TABLE 5
Comparative evaluation of different hMPV primers for detection of hMPV strains

| | | | | | | | | | | | L | |
|---|---|---|---|---|---|---|---|---|---|---|---|---|
| | | | N | | M | | F | | P | | primers | |
| | | | Primers | | primers | | primers | | primers | | SEQ. ID. Nos. | |
| | Type of | | SEQ. ID. Nos. | | SEQ. ID. Nos. | | SEQ. ID. Nos. | | SEQ. ID. Nos. | | 72 + 73 | |
| | sample | | 92 + 94 | | 100 + 101 | | 97 + 98 | | 102 + 103 | | 112 + 113 | |
| Strains No. | I or S | | +/− | Tm (° C.) | +/− | Tm (° C.) | +/− | Tm (° C.) | +/− | Tm (° C.) | +/− | Tm (° C.) |
| C-74501 | I | 2000 | + | 82.92 | − | | + | 83.65 | − | | + | 78.14 |
| G-74783 | I | 2000 | + | 82.76 | + | 83.49 | − | | + | 81.51 | + | 78.20 |
| C-76837 | I | 2000 | + | 82.94 | + | 83.43 | + | 83.17 | + | 80.86 | − | |
| C-77719 | I | 2001 | + | 83.26 | + | 84.64 | − | | − | | + | 77.87 |
| C-77738 | I | 2001 | + | 82.99 | − | | − | | − | | + | 77.51 |
| C-77748 | I | 2001 | + | 83.06 | + | 82.96 | + | 83.10 | + | 81.27 | + | 77.38 |
| C-77807 | I | 2001 | + | 82.99 | + | 83.26 | + | 83.25 | + | 80.65 | + | 78.34 |
| C-78024 | I | 2001 | + | 82.86 | + | 83.95 | − | | − | | − | |
| C-78028 | I | 2001 | + | 82.98 | + | 83.13 | − | | − | | + | 78.32 |
| C-78123 | I | 2001 | + | 82.83 | − | | − | | − | | + | 78.48 |
| C-85984 | I | 2002 | + | 82.59 | + | 83.21 | + | 83.51 | + | 79.99 | + | 77.97 |
| C-85448 | I | 2002 | + | 83.66 | + | 82.72 | − | | + | 80.31 | + | 78.88 |
| C-85983 | I | 2002 | + | 82.59 | + | 83.03 | + | 83.88 | + | 80.46 | + | 78.01 |
| C-85988 | I | 2002 | + | 82.17 | − | | + | 84.39 | − | | + | 78.06 |
| C-86316 | I | 2002 | + | 82.64 | + | 82.75 | + | 83.18 | + | 80.18 | + | 78.02 |
| C-85126 | I | 2002 | + | 81.89 | + | 82.05 | + | 82.39 | + | 80.00 | + | 78.04 |
| C-85473 | I | 2002 | + | 82.39 | + | 82.57 | + | 82.91 | + | 80.74 | + | 77.82 |
| C-85332 | I | 2002 | + | 81.98 | − | | − | | − | | + | 77.72 |
| C-85596 | I | 2002 | + | 82.24 | + | 82.73 | + | 83.17 | + | 80.35 | + | 77.58 |
| C-85542 | I | 2002 | + | 82.22 | + | 82.72 | − | | + | 80.08 | + | 77.67 |
| HMPV-118 | S | 2002 | + | 82.47 | − | | − | | − | | ND | ND |
| HMPV-174 | S | 2002 | + | 82.18 | + | 82.06 | + | 83.16 | + | 80.17 | + | 78.20 |
| HMPV-176 | S | 2002 | + | 81.41 | − | | + | 83.97 | − | | + | 77.81 |
| HMPV-193 | S | 2002 | + | 82.22 | + | 81.89 | + | 83.00 | + | 80.58 | + | 77.94 |
| HMPV-194 | S | 2002 | + | 82.24 | + | 81.32 | + | 82.98 | − | | + | 77.84 |
| HMPV-198 | S | 2002 | + | 82.66 | − | | − | | − | | − | |
| HMPV-200 | S | 2002 | + | 82.53 | − | | + | 83.46 | − | | + | 77.62 |
| HMPV-208 | S | 2002 | + | 82.11 | + | 81.81 | + | 83.33 | + | 80.06 | + | 78.40 |
| HMPV-217 | S | 2002 | + | 82.13 | + | 81.58 | + | 83.01 | − | | + | 77.76 |
| HMPV-223 | S | 2002 | + | 81.89 | + | 82.36 | + | 83.04 | − | | + | 78.10 |

| | | | | | |
|---|---|---|---|---|---|
| Mean | 82.63 ± 0.87 | 82.75 ± 0.80 | 83.29 ± 0.45 | 80.48 ± 0.46 | 77.99 ± 0.33 |

Note:
I = viral isolates;
S = clinical specimens;
Tm = melting temperature as determined by a LightCycler instrument.

TABLE 6
Size of the viral fusion (F) gene fragments obtained for different
hMPV isolates following enzymatic digestion with restriction
endonucleases.

| | Fragments obtained after digestion | | |
|---|---|---|---|
| | with the restriction endonuclease | | |
| SEQ ID NO. | ApaL I | Avr II | F genotype^{a} |
| 35 | 532 bp + 227 bp | 759 bp | I |
| 58 | 532 bp + 227 bp | 759 bp | I |
| 39 | 759 bp | 447 bp + 312 bp | II |
| 40 | 532 bp + 227 bp | 759 bp | I |
| 41 | 532 bp + 227 bp | 759 bp | I |
| 43 | 759 bp | 447 bp + 312 bp | II |
| 44 | 532 bp + 227 bp | 759 bp | I |
| 59 | 532 bp + 227 bp | 759 bp | I |
| 45 | 532 bp + 227 bp | 759 bp | I |

^{a}As determined by DNA sequencing and phylogenetic studies.

TABLE 7
Type of laboratory confirmation by type of infection.

| | hMPV | | hRSV | | Influenza A | | Adenovirus | | PIV 2 | |
|---|---|---|---|---|---|---|---|---|---|---|
| | Tests | | Tests | | Tests | | Tests | | Tests | |
| Laboratory test | done | Positive | Done | Positive | done | Positive | done | Positive | done | Positive |
| PCR | 208 | 12 | 208 | 106 | 208 | 45 | NA | NA | NA | NA |
| | | 5.8% | | 51.0% | | 21.6% | | | | |
| Culture | 100 | 2 | 100 | 31 | 100 | 14 | 100 | 6 | 100 | 1 |
| | | 2% | | 31% | | 14% | | 6% | | 1% |
| Antigen detection | NA | NA | NA | NA | NA | NA | 81 | 1 | 76 | 1 |
| (Immunofluorescence) | | | | | | | | 1% | | 1.3% |
| Antigen detection | NA | NA | 204 | 94 | 100 | 14 | NA | NA | NA | NA |
| (ELISA) | | | | 46.1% | | 14% | | | | |

| | | | | | |
|---|---|---|---|---|---|
| Total (+) in at least one test | (5.8%) | 118 (56.7%) | 49 (23.6%) | 6 (6%) | 2 (2%) |
| Delay between onset of | 6.3/5.0 | 5.2/4.0 | 8.7/5.0 | 6.0/6.5 | 3.0/3.0 |
| symptoms and NPA, days | | | | | |
| Mean/median | | | | | |

Note:
hMPV, human metapneumovirus;
hRSV, human respiratory syncytial virus;
PIV, parainfluenza virus;
NPA, nasopharyngeal aspirate;
NA, not available.

TABLE 8
Signs and symptoms by type of viral infection.

| | | | | | Multiple | No virus | |
|---|---|---|---|---|---|---|---|
| Signs and | HMPV | hRSV | Influenza A | Single virus | viruses | detected | Total |
| symptoms | N = 12 | N = 118 | N = 49 | N = 141 | N = 23 | N = 44 | N = 208 |
| Fever | 8 | 67 | 38 | 85 | 17 | 25 | 127 |
| | 67% | 57% | 78% | 60% | 74% | 57% | 61% |
| Cough | 12 | 117 | 47 | 138 | 23 | 40 | 201 |
| | 100% | 99% | 96% | 98% | 100% | 90% | 97% |
| Rhinorrhea | 11 | 107 | 41 | 123 | 22 | 42 | 187 |
| | 92% | 91% | 84% | 87% | 96% | 96% | 90% |
| Retractions | 11 | 112 | 40 | 125 | 22 | 39 | 186 |
| | 92% | 95% | 82% | 89% | 96% | 89% | 89% |
| Wheezing | 10 | 77 | 28 | 83 | 19 | 31 | 133 |
| | 83% | 65% | 57% | 59% | 83% | 71% | 64% |
| Lacrymation | 3 | 37 | 15 | 46 | 6 | 11 | 63 |
| | 25% | 31% | 31% | 33% | 26% | 25% | 30% |
| Diarrhea | 1 | 19 | 13 | 24 | 5 | 10 | 39 |
| | 8% | 17% | 27% | 17% | 22% | 23% | 19% |
| Vomiting | 3 | 9 | ‘5 | 10 | 4 | 1 | 15 |
| | 25% | 8% | 10% | 7% | 17% | 2% | 7% |
| Other | 0 | 31 | 9 | 33 | 4 | 9 | 46 |
| | | 26% | 18% | 23% | 17% | 21% | 22% |

Note:
hMPV, human metapneumovirus;
hRSV, human respiratory syncytial virus.

TABLE 9
Final diagnoses (complications) by type of viral infection.

| | | | | | Multiple | No virus | |
|---|---|---|---|---|---|---|---|
| Complications | hMPV | HRSV | Influenza A | Single virus | viruses | detected | Total |
| | N = 12 | N = 118 | N = 49 | N = 141 | N = 23 | N = 44 | N = 208 |
| Bronchiolitis | 8 | 99 | 25 | 98 | 19 | 25 | 142 |
| | 66.7% | 83.9% | 51.0% | 69.5% | 82.6% | 56.8% | 68.3% |
| Otitis | 6 | 70 | 27 | 78 | 15 | 24 | 117 |
| | 50.0% | 59.3% | 55.1% | 55.3% | 65.2% | 54.6% | 56.3% |
| Pneumonia | 2 | 30 | 18 | 39 | 7 | 12 | 58 |
| | 16.7% | 25.4% | 36.7% | 27.7% | 30.4% | 27.3% | 27.9% |
| Laryngotracheobroachitis | 0 | 10 | 6 | 9 | 4 | 1 | 14 |
| | | 8.5% | 12.2% | 6.4% | 17.4% | 2.3% | 6.7% |
| Sinusitis | 0 | 3 | 3 | 2 | 2 | 1 | 5 |
| | | 2.5% | 6.1% | 1.4% | 8.7% | 2.3% | 2.4% |
| Pharyngitis | 0 | 1 | 0 | 2 | 0 | 2 | 4 |
| | | 0.9% | | 1.4% | | 4.6% | 1.9% |
| Flu syndrome | 0 | 2 | 0 | 2 | 0 | 4 | 6 |
| | | 1.7% | | 1.4% | | 9.1% | 2.9% |
| Croup | 0 | 2 | 0 | 2 | 0 | 1 | 3 |
| | | 1.7% | | 1.4% | | 2.3% | 1.4% |
| Other | 1 | 4 | 3 | 9 | 0 | 5 | 14 |
| | 8.3% | 3.4% | 6.1% | 6.4% | | 11.4% | 6.7% |

Note:
hMPV, human metapneumovirus;
hRSV, human respiratory syncytial virus.

TABLE 10
List of PCR primers used for amplification of respiratory viruses

| Primer | | | Originating DNA | |
|---|---|---|---|---|
| SEQ ID NO. | Virus | Target Gene | Position^{a} | SEQ ID NO. |
| 139 | Influenza A | Matrix | 32/7 | 130-131 |
| 140 | Influenza A | Matrix | 276^{b}/251^{b} | 130-131 |
| 141 | Influenza B | Matrix | 82 | 132 |
| 142 | Influenza B | Matrix | 605^{b} | 132 |
| 143 | RSV | Fusion | 1111 | 133 |
| 144 | RSV | Fusion | 1490^{b} | 133 |
| 95 | hMPV | Nucleocapsid | 58 | 9 |
| 96 | hMPV | Nucleocapsid | 986^{b} | 9 |
| 110 | hMPV | Polymerase | 142 | 75 |
| 111 | hMPV | Polymerase | 691^{b} | 75 |
| 145 | PIV-1 | Nucleocapsid | 64 | 134 |
| 146 | PIV-1 | Nucleocapsid | 147^{b} | 134 |
| 149 | PIV-3 | Nucleocapsid | 416 | 135 |
| 150 | PIV-3 | Nucleocapsid | 649^{b} | 135 |
| 154 | Adenovirus | Hexon | 154 | 136 |
| 155 | Adenovirus | Hexon | 368^{b} | 136 |
| 160 | Entero/Rhinovirus | 5′-non coding region | 94/71 | 137/138 |
| 161 | Entero/Rhinovirus | 5′-non coding region/VP2 | 746^{b}/603^{b} | 137/138 |
| 141 | IC^{c} multiplex 1 | DNA polymerase HSV-2 | 1 | 170 |
| 142 | IC^{c} multiplex 1 | DNA polymerase HSV-2 | 558^{b} | 170 |
| 160 | IC^{c} multiplex 2 | DNA polymerase HSV-2 | 1 | 171 |
| 161 | IC^{c} multiplex 2 | DNA polymerase HSV-2 | 554^{b} | 171 |
| 112 | hMPV | Polymerase | 7308 | 1 |
| 114 | hMPV | Polymerase | 7616 | 1 |
| 147 | PIV-1 | Nucleocapsid | 822 | 134 |
| 148 | PIV-1 | Nucleocapsid | 1029 | 134 |
| 151 | PIV-3 | Nucleocapsid | 792 | 135 |
| 152 | PIV-3 | Nucleocapsid | 902 | 135 |
| 153 | Adenovirus | Hexon | 7 | 136 |
| 156 | Sars-CoV | Polymerase | | |
| 157 | Sars-CoV | Polymerase | | |
| 158 | Sars-CoV | Polymerase | | |
| 159 | Sars-CoV | Polymerase | | |

^{a}Position refers to nucleotide position of 5′-end of primer. Oligonucleotides are 16-26 bp in length.
^{b}Primer is reverse-complement of target sequence.
^{c}IC refers to the internal control used to monitor the efficiency of the PCR amplification

TABLE 11
List of viral probes developed in the present invention.

| | |
|---|---|
| Probe | Originating DNA |

| SEQ ID NO. | Target | Position^{a} | SEQ ID NO. |
|---|---|---|---|
| 162 | Influenza A | 114^{b}/89^{b} | 130/131 |
| 163 | Influenza B | 383 | 132 |
| 164 | hRSV | 1391^{b} | 133 |
| 128 | hMPV | 824 | 9 |
| 129 | hMPV | 434 | 75 |
| 165 | PIV-1 | 96 | 134 |
| 166 | PIV-3 | 616^{b} | 135 |
| 167 | Adenovirus | 228 | 136 |
| 168 | Rhinovirus | 467^{b} | 138 |
| 169 | Enterovius | 644^{b} | 137 |
| 172 | Internal Control^{c} | 321^{b}/325^{b} | 170/171 |

^{a}Position refers to nucleotide position of 5′-end of primer. Oligonucleotides are 20-23 bp in length.
^{b}Probe is reverse-complement of target sequence.
^{c}A modified DNA polymerase gene sequence of herpes simplex virus (HSV) type 2 serves as the internal control template.

TABLE 12
Length of amplicons obtained with the different viral primer pairs.

| Primer | | | Length |
|---|---|---|---|
| SEQ ID NO. | Virus | Target gene | of amplicons |
| 139 + 140 | Influenza A | Matrix | 245 bp |
| 141 + 142 | Influenza B | Matrix | 524 bp |
| 143 + 144 | hRSV | Fusion | 380 bp |
| 95 + 96 | hMPV | Nucleocapsid | 929 bp |
| 110 + 111 | hMPV | Polymerase | 550 bp |
| 145 + 146 | PIV-1 | Nucleocapsid | 84 bp |
| 149 + 150 | PIV-3 | Nucleocapsid | 234 bp |
| 154 + 155 | Adenoviruses | Hexon | 215 bp |
| 160 + 161 | Rhinoviruses | 5′-non coding region - VP2 | 533 bp |
| 160 + 161 | Enteroviruses | 5′-non coding region | 653 bp |
| 141 + 142 | IC multiplex 1 | DNA polymerase HSV-2 | 558 bp |
| 160 + 161 | IC multiplex 2 | DNA polymerase HSV-2 | 554 bp |
| 112 + 114 | hMPV | Polymerase | 308 bp |
| 147 + 148 | PIV-1 | Nucleocapsid | 208 bp |
| 151 + 152 | PIV-3 | Nucleocapsid | 111 bp |
| 153 + 155 | Adenovirus | Hexon | 362 bp |
| 156 + 157 | Sars-CoV | Polymerase | 198 bp |
| 158 + 159 | Sars-CoV | Polymerase | 368 bp |

TABLE 13
Selection of primer sets with resulting melting temperature (Tm)
values of the corresponding amplicons for each multiplex assay.

| Primer | | | |
|---|---|---|---|
| SEQ ID NO. | Target | Multiplex NO. | Tm (° C.) |
| 139 + 140 | Influenza A | 1 | 85 |
| 141 + 142 | Influenza B | 1 | 83 |
| 143 + 144 | hRSV | 1 | 79 |
| 95 + 96 or | hMPV | 1 | 82 or 77 |
| 112 + 114 | | | |
| 141 + 142 | Internal Control | 1 | 91 |
| 147 + 148 | Parainfluenza 1 | 2 | 80.0 |
| 151 + 152 | Parainfluenza 3 | 2 | 77.5 |
| 153 + 155 | Adenovirus | 2 | 89.0 |
| 160 + 161 | Rhinovirus | 2 | 82.0 |
| 160 + 161 | Enterovirus | 2 | 85.0 |
| 160 + 161 | Internal Control | 2 | 91 |

TABLE 14
Evaluation of the multiplex PCR respiratory assay with temporally-
different strains of influenza (Flu) and human respiratory syncytial
(HRSV) viruses.

| | Subtype/ | | | | Tm |
|---|---|---|---|---|---|
| Virus | Genotype^{a} | Year | Type | Strain | (° C.) |
| Flu A | H1 | 99-00 | clinical | — | 85.10 |
| Flu A | H1 | 99-00 | clinical | — | 85.39 |
| Flu A | H1 | 00-01 | clinical | — | 85.12 |
| Flu A | H1 | 00-01 | clinical | — | 85.20 |
| Flu A | H1 | 00-01 | clinical | — | 85.04 |
| Flu A | H1 | 01-02 | clinical | — | 84.62 |
| Flu A | H1 | 01-02 | clinical | — | 85.07 |
| Flu A | H1 | 02-03 | clinical | — | 85.50 |
| Flu A | H1 | 02-03 | clinical | — | 85.29 |
| Flu A | H1 | 95 | vaccine | A/Beijing/ | 85.06 |
| | | | | 262/95 | |
| Flu A | H1 | 33 | vaccine | A/WSN/33 | 85.25 |
| Mean | | | | | 85.15 ± 0.22 |
| AH1 | | | | | |
| Flu A | H3 | 98-99 | clinical | — | 85.58 |
| Flu A | H3 | 98-99 | clinical | — | 85.18 |
| Flu A | H3 | 98-99 | clinical | — | 85.35 |
| Flu A | H3 | 98-99 | clinical | — | 85.57 |
| Flu A | H3 | 98-99 | clinical | — | 85.37 |
| Flu A | H3 | 01-02 | clinical | — | 85.07 |
| Flu A | H3 | 01-02 | clinical | — | 85.65 |
| Flu A | H3 | 02-03 | clinical | — | 84.92 |
| Flu A | H3 | 99 | vaccine | A/Panama/ | 85.18 |
| | | | | 2007/99 | |
| Flu A | H3 | 97 | vaccine | A/Sydney/5/97 | 85.06 |
| Mean | | | | | 85.31 ± 0.25 |
| AH3 | | | | | |
| Mean | | | | | 85.27 ± 0.22 |
| FluA | | | | | |
| Flu B | — | 98-99 | clinical | — | 83.87 |
| Flu B | — | 98-99 | clinical | — | 83.12 |
| Flu B | — | 00-01 | clinical | — | 83.01 |
| Flu B | — | 00-01 | clinical | — | 83.03 |
| Flu B | — | 00-01 | clinical | — | 82.88 |
| Flu B | — | 00-01 | clinical | — | 83.75 |
| Flu B | — | 00-01 | clinical | — | 83.17 |
| Flu B | — | 02-03 | clinical | — | 83.89 |
| Flu B | — | 02-03 | clinical | — | 83.84 |
| Flu B | — | 97 | vaccine | B/Harbin/ | 84.12 |
| | | | | 7/97 | |
| Mean | | | | | 83.47 ± 0.46 |
| FluB | | | | | |
| HRSV | B | 99-00 | clinical | — | 79.32 |
| HRSV | A | 99-00 | clinical | — | 79.83 |
| HRSV | A | 00-01 | clinical | — | 79.06 |
| HRSV | A | 00-01 | clinical | — | 78.85 |
| HRSV | B | 01-02 | clinical | — | 79.34 |
| HRSV | B | 01-02 | clinical | — | 79.52 |
| HRSV | B | 01-02 | clinical | — | 79.98 |
| HRSV | B | 01-02 | clinical | — | 79.52 |
| HRSV | A | 01-02 | clinical | — | 79.84 |
| HRSV | B | 01-02 | clinical | — | 79.42 |
| HRSV | B | 01-02 | clinical | — | 79.49 |
| HRSV | B | 01-02 | clinical | — | 79.48 |
| HRSV | B | 01-02 | clinical | — | 79.55 |
| HRSV | B | 01-02 | clinical | — | 79.32 |
| HRSV | A | 01-02 | clinical | — | 79.77 |
| HRSV | A | 01-02 | clinical | — | 79.92 |
| HRSV | A | 01-02 | clinical | — | 79.40 |
| Mean | | | | | 79.52 ± 0.43 |
| HRSV-A | | | | | |
| Mean | | | | | 79.49 ± 0.19 |
| HRSV-B | | | | | |
| Mean | | | | | 79.51 ± 0.30 |
| HRSV | | | | | |

^{a}Subtyping was performed by multiplex PCR for the influenza hemagglutinin gene (2) whereas genotyping was done by conventional PCR for the HRSV G gene (22)

TABLE 15
Evaluation of the multiplex PCR respiratory assay for detection
of influenza A (Flu A) virus.

| | | Antigenic test | | Flu A M2 | |
|---|---|---|---|---|---|
| | | (Directigen Flu A + B) | | PCR assay^{a} | |
| | | (n = 172) | | (n = 27) | |
| | | + | − | + | − |
| Multiplex PCR | + | 17 | 25 | 22 | 3 |
| | − | 2 | 128 | 0 | 2 |

Sensitivity: 39/39 = 100%
Specificity: 130/133 = 97.7%
Positive predictive value: 39/42 = 92.8%
Negative predictive value: 130/130 = 100%
^{a}Only performed for samples showing discordant results with the multiplex PCR assay and the antigenic test.

TABLE 16
Evaluation of discordant test results for influenza A (Flu A) virus.

| | Multiplex PCR | Antigenic | | Other PCR- |
|---|---|---|---|---|
| Sample | Flu A results | test | M2 PCR | amplified |
| No. | (C_{τ})^{a} | Flu A results | Flu A results | viruses^{b} |
| 16 | — | + | − | HRSV |
| 23 | +(27) | − | + | |
| 24 | — | + | − | HRSV |
| 27 | +(28) | − | + | HRSV |
| 35 | +(28) | − | + | HRSV |
| 36 | +(32) | − | + | |
| 40 | +(34) | − | + | |
| 43 | +(36) | − | + | HRSV |
| 44 | +(35) | − | + | |
| 50 | +(34) | − | + | |
| 51 | +(33) | − | + | HRSV |
| 103 | +(35) | − | + | |
| 107 | +(35) | − | + | |
| 114 | +(37) | − | − | |
| 118 | +(38) | − | + | HRSV |
| 120 | +(36) | − | + | HRSV |
| 125 | +(38) | − | + | |
| 130 | +(35) | − | + | HRSV |
| 131 | +(36) | − | − | |
| 132 | +(24) | − | + | HRSV |
| 134 | +(36) | − | − | |
| 141 | +(28) | − | + | |
| 152 | +(34) | − | + | |
| 158 | +(32) | − | + | |
| 160 | +(33) | − | + | HRSV |
| 161 | +(28) | − | + | HRSV |
| 163 | +(32) | − | + | |

^{a}Cycle threshold value as determined by LightCycler.
^{b}Viruses amplified by the multiplex PCR assay i.e. influenza (Flu) A and B as well as the human respiratory syncytial virus (HRSV).

TABLE 17
Evaluation of the multiplex PCR respiratory assay for
detection of human respiratory syncytial virus (HRSV).

| | | Antigenic test | | HRSV gG | |
|---|---|---|---|---|---|
| | | (RSV TestPack) | | PCR assays^{a} | |
| | | (n = 204) | | (n = 32) | |
| | | + | − | + | − |
| Multiplex | + | 83 | 21 | 20 | 1 |
| PCR | | | | | |
| | − | 11 | 89 | 6 | 5 |

Sensitivity: 103/109 = 94.5%
Specificity: 94/95 = 98.9%
Positive predictive value: 103/104 = 99.0%
Negative predictive value: 94/100 = 94.0%
^{a}Only performed for samples showing discordant results with the multiplex PCR assay and the antigenic test.

TABLE 18
Evaluation of discordant test results for
human respiratory syncytial virus (HRSV).

| | | Antigenic | Glycoprotein G | |
|---|---|---|---|---|
| | Multiplex PCR | test | PCR HRSV | Other PCR- |
| Sample | HRSV results | HRSV | results | amplified |
| No. | (C_{τ})^{a} | results | (genotype) | viruses^{b} |
| 16 | +(32) | − | +(B) | |
| 17 | +(27) | − | +(B) | |
| 26 | +(36) | − | +(B) | |
| 34 | — | + | − | |
| 35 | +(33) | − | +(B) | Flu A |
| 43 | +(36) | − | +(A) | Flu A |
| 46 | +(34) | − | +(B) | |
| 55 | +(34) | − | +(B) | |
| 58 | +(33) | − | +(B) | |
| 62 | — | + | +(A) | Flu A |
| 70 | +(37) | − | +(B) | |
| 71 | +(35) | − | +(B) | |
| 72 | +(34) | − | +(B) | |
| 73 | +(26) | − | +(A) | |
| 79 | +(33) | − | +(B) | |
| 80 | — | + | − | |
| 91 | +(35) | − | +(A) | |
| 95 | +(29) | − | +(B) | |
| 100 | +(30) | − | +(A) | |
| 112 | — | + | − | |
| 129 | +(34) | − | +(B) | |
| 130 | +(35) | − | +(B) | Flu A |
| 134 | — | + | +(A) | Flu A |
| 139 | — | + | +(A) | |
| 142 | — | + | +(A) | Flu A |
| 144 | — | + | +(B) | Flu A |
| 150 | +(34) | − | − | |
| 187 | +(31) | − | +(A) | |
| 191 | +(29) | − | +(A) | |
| 193 | — | + | +(A) | |
| 211 | — | + | − | |
| 212 | — | + | − | |

^{a}Cycle threshold value as determined by Lightcycler.
^{b}Viruses amplified by the multiplex PCR assay i.e. influenza (Flu) A and B as well as the human respiratory syncytial virus (HRSV).

1- A method for detecting and/or quantifying human metapneumovirus (hMPV) in a sample said method comprising:
providing at least one probe or primer specific for a nucleic acid sequence of said hMPV;
contacting said at least one probe and/or primer with said sample to allow annealing of said probe and/or primer with said nucleic acid sequence; and
detecting and/or quantifying said nucleic acid sequence using said annealed probe and/or primer.

2- The method as claimed in claim 1 wherein said nucleic acid sequence is comprised in a hMPV gene selected from a group consisting of nucleocapsid (N), phosphoprotein (P), matrix (M), fusion (F), membrane (M2), polymerase (L), glycoprotein (G) and small hydrophobic (SH) genes.

3- The method as claimed in claim 2 wherein said nucleic acid sequence is selected from any one of SEQ ID NO 2-8, 10-74, 76-90 or fragments thereof.

4- The method as claimed in claim 3 wherein said probe or primer is at least 10 nucleotides in length.

5- A method for detecting and/or quantifying human metapneumovirus (hMPV) in a sample said method comprising:
performing reverse transcription using a template RNA derived from said sample to obtain a nucleic acid product;
amplifying said nucleic acid product using a pair of oligonucleotide primers wherein said primers are specific for a sequence comprised in a hMPV gene selected from a group consisting of nucleocapsid (N), phosphoprotein (P), matrix (M), fusion (F), membrane (M2), polymerase (L), glycoprotein (G) and small hydrophobic (SH) genes to produce an amplification product; and
detecting and/or quantifying said amplification product.

6- The method as claimed in claim 5 wherein said oligonucleotide primers are at least 10 nucleotides in length and capable of hybridizing with any one of SEQ ID 2-8, 10-74, 76-90.

7- The method as claimed in claim 5 or 6 wherein said step of performing reverse transcription comprises contacting said template RNA with a primer selected from SEQ ID Nos 91-114 and wherein said oligonucleotide primers for PCR are selected from SEQ ID Nos 91-114.

8- The method as claimed in any one of claim 5 to 7 wherein said oligonucleotide primers are pairs of primers selected from SEQ ID Nos 91 and 94, 92 and 94, 91 and 93, 97 and 99, 97 and 98, 100 and 101, 102 and 103, 104 and 107, 105 and 106, 108 and 109, 112 and 113 and 112 and 114.

9- The method as claimed in claim 5 wherein said template RNA is contacted with primer selected from SEQ ID NO 91-94 and 112-114 and wherein said oligonucleotide primers are selected from SEQ ID NO 91-94 and 112-114.

10- The method as claimed in any one of claim 5-9 wherein said step of amplifying is performed using PCR.

11- The method as claimed in claim 10 wherein said PCR is RT-PCR.

12- The method as claimed in claim 11 wherein said RT-PCR is real time RT-PCR.

13- The method as claimed in claim 11 or 12 wherein said RT-PCR comprises detecting said amplification products by characteristic melting temperature of said amplification products.

14- The method as claimed in claim 11 or 12 wherein said RT-PCR comprises detecting and/or quantifying said amplification products with probes specific for said products.

15- The method as claimed in claim 14 wherein said probes are selected from SEQ ID no 115-129.

16- A composition for detecting a hMPV said composition comprising a combination of oligonucleotides for performing reverse transcription of a template RNA from said hMPV virus and for an amplification reaction of a product obtained from said reverse transcription wherein said oligonucleotides for said amplification reaction are specific for a sequence comprised in a hMPV gene selected from a group consisting of nucleocapsid (N), phosphoprotein (P), matrix (M), fusion (F), membrane (M2), polymerase (L), glycoprotein (G) and small hydrophobic (SH) genes.

17- The composition of claim 16 wherein said oligonucleotides for said amplification reaction are primers selected from the group comprising SEQ ID Nos 91-114 and combination thereof.

18- A kit comprising a combination of oligonucleotides wherein the oligonucleotides are selected from SEQ ID NOs: 91-114 and the pairs of SEQ ID Nos 91 and 94, 92 and 94, 91 and 93, 97 and 99, 97 and 98, 100 and 101, 102 and 103, 104 and 107, 105 and 106, 108 and 109, 112 and 113 and 112 and 114.

19- A method for typing an hMPV strain which comprises the steps of:
amplifying an hMPV nucleic acid fragment and detecting specific viral sequences;
digesting said fragment with one or more restriction endonuclease; and
analysing the restriction fragment length polymorphisms.

20- The method as claimed in claim 19 wherein said nucleotide sequences are found in fusion (F) or glycoprotein (G) gene.

21- The method as claimed in claim 20 wherein said sequences are selected from SEQ ID NO 26-74.

22- A method for detecting and/or quantifying two or more respiratory viruses in a sample said method comprising:
providing at least two probes and/or primers specific for a nucleic acid sequences in said two or more respiratory viruses;
contacting said at least two probes and/or primers with said sample to allow annealing of said probes and/or primers with said nucleic acid sequences; and
detecting and/or quantifying said nucleic acid sequences using said annealed probes and/or primers.

23- The method as claimed in claim 22 wherein at least one of said nucleic acid sequences is comprised in a hMPV gene selected from a group consisting of nucleocapsid (N), phosphoprotein (P), matrix (M), fusion (F), membrane (M2), polymerase (L), glycoprotein (G) and small hydrophobic (SH) genes.

24- The method as claimed in claim 23 wherein said nucleic acid sequence is selected from any one of SEQ ID NO 2-8, 10-74, 76-90 or fragments thereof.

25- The method as claimed in claim 24 wherein said probes and/or primers are at least 10 nucleotides in length.

26- A method for detecting and/or quantifying two or more respiratory viruses in a sample said method comprising:
performing reverse transcription using a template RNA derived from said sample to obtain nucleic acid products for each of said viruses;
amplifying said nucleic acid products using two or more pairs of oligonucleotide primers to produce an amplification product; and
detecting and/or quantifying said amplification products.

27- The method as claimed in claim 26 wherein at least one of said primers is specific for a sequence comprised in a hMPV gene selected from a group consisting of nucleocapsid (N), phosphoprotein (P), matrix (M), fusion (F), membrane (M2), polymerase (L), glycoprotein (G) and small hydrophobic (SH) genes.

28- The method as claimed in claim 27 wherein said oligonucleotide primers specific for a sequence comprised in a hMPV gene are capable of hybridizing with any one of SEQ ID 2-8, 10-74, 76-90.

29- The method as claimed in claim 28 wherein said oligonucleotide primers are at least 10 nucleotides in length.

30- The method as claimed in claim 29 wherein said oligonucleotide primer specific for a sequence comprised in a hMPV gene are selected from SEQ ID Nos 91-114.

31- The method as claimed in any one of claim 26-30 wherein said at least two respiratory viruses comprise influenza A and B, human respiratory syncytial viruses A and B, parainfluenza viruses (PIV) types 1-4, adenoviruses, rhinoviruses, enteroviruses and coronaviruses or any combination thereof and wherein said primers and said two or more viruses are selected such that the detection and or quantification is specific for each of said viruses.

32- The method as claimed in claim 31 wherein said oligonucleotide primers are at least 10 nucleotides in length and capable of hybridizing with any one of SEQ ID NO 130-138 and 170-171.

33- The method as claimed in claim 31 or 32 wherein said at least two viruses are influenza A, human Respiratory syncytial virus, human metapneumovirus and influenza B.

34- The method as claimed in claim 31 or 32 wherein said at least two viruses are Parainfluenza 1, Rhinovirus, Adenovirus, Parainfluenza 3, Enterovirus, and coronavirus.

35- The method as claimed in claim 31 wherein said pairs of oligonucleotide primers are selected from SEQ ID Nos 139 and 140, 141 and 142, 143 and 144, 92 and 94, 112 and 113, 112 and 114, 145 and 146, 147 and 148, 149 and 150, 151 and 151, 154 and 155, 153 and 155, 156 and 157, 158 and 159, 160 and 161.

36- The method as claimed in any one of claim 26-35 wherein said step of amplifying is performed using PCR and said primer are selected such that the products of the amplifications can be distinguished.

37- The method as claimed in claim 36 wherein said PCR is RT-PCR.

38- The method as claimed in claim 37 wherein said RT-PCR is real time RT-PCR.

39- The method as claimed in claim 37 or 38 wherein said RT-PCR comprises detecting said amplification products by characteristic melting temperature of said amplification products.

40- The method as claimed in claim 37 or 38 wherein said RT-PCR comprises detecting and/or quantifying said amplification products with probes specific for said products.

41- The method as claimed in claim 40 wherein said probes are selected from SEQ ID no 162-169 and 172.

42- A composition for detecting at least two respiratory viruses said viruses comprising at least hMPV said composition comprising a combination of oligonucleotides for performing reverse transcription of a template RNA from said at least two respiratory viruses and for an amplification reaction of a product obtained from said reverse transcription wherein said oligonucleotides for said amplification reaction comprise oligonucleotides specific for a sequence comprised in a hMPV gene selected from a group consisting of nucleocapsid (N), phosphoprotein (P), matrix (M), fusion (F), membrane (M2), polymerase (L), glycoprotein (G) and small hydrophobic (SH) genes.

43- The composition of claim 42 wherein said oligonucleotides for said amplification reaction are primers selected from the group comprising SEQ ID Nos 91-114 and combination thereof.

44- A method for simultaneously detecting two or more respiratory tract viruses in a sample, said method comprising:
selecting primers specific for a nucleic acid sequence in each of said two or more viruses, said primers to be used in an amplification reaction for detection of said nucleic acid sequences; and
distributing said primers in one or more assay mixture comprising said sample and at least two pairs of primers;
wherein said selecting and said distributing is based on said detection of said nucleic acid sequence of said two or more viruses being substantially free of overlap and enabling to distinguish between said two or more viruses.

45- The method as claimed in claim 44 wherein said amplification reaction is PCR.

46- The method as claimed in claim 45 wherein said PCR is RT-PCR.

47- The method as claimed in claim 45 wherein said RT-PCR is real time RT-PCR.

48- The method as claimed in claim 24 wherein said detection is melting temperature.

49- An isolated nucleic acid comprising a sequence that hybridizes under stringent conditions to a hybridization probe the nucleotide sequence of which consists of any one of SEQ ID NO 2-8, 10-74, 76-90 or complements thereof.

50- A kit comprising a nucleic acid probe that is at least 10 nucleotides in length and is selected from a group consisting sequences hybridizable to SEQ ID Nos 2-8, 10-74, 76-90 or fragments thereof.

51- A human metapneumovirus (hMPV) deposited with the International Depository Authority of Canada under Accession number IDAC 111203-01 (CAN97-83) comprising nucleic acid sequence SEQ ID NO 173.

52- A human metapneumovirus (hMPV) deposited with the International Depository Authority of Canada under Accession number IDAC 111203-02 (CAN98-75) comprising nucleic acid sequence SEQ ID NO 174.


SEQUENCE LISTING (174 sequences)

SEQ ID NO: 1 - DNA, 13350 bp, human metapneumovirus
  gene (1)...(13350): human metapneumovirus, strain 00-1 Accession number af371337
gtataaattagattccaaaaaaatatgggacaagtgaaaatgtctcttcaagggattcacctgagtgatttatcatacaagcatgctatattaaaagagtctcagtacacaataaaaagagatgtgggtacaacaactgcagtgacaccctcatcattgcaacaagaaataacactgttgtgtggagaaattctgtatgctaaacatgctgactacaaatatgctgcagaaataggaatacaatatattagcacagctttaggatcagagagagtgcagcagattctgaggaactcaggcagtgaagtccaagtggtcttaaccagaacgtactctctggggaaaattaaaaacaataaaggagaagatttacagatgttagacatacacggggtagagaagagctgggtagaagagatagacaaagaagcaaggaaaacaatggcaaccttgcttaaggaatcatcaggtaatatcccacaaaatcagaggccctcagcaccagacacacccataatcttattatgtgtaggtgccttaatattcactaaactagcatcaaccatagaagtgggactagagaccacagtcagaagggctaaccgtgtactaagtgatgcactcaagagataccctagaatggacataccaaagattgccagatccttctatgacttatttgaacaaaaagtgtatcacagaagtttgttcattgagtatggcaaagcattaggctcatcatctacaggcagcaaagcagaaagtctatttgttaatatattcatgcaagcttatggggccggtcaaacaatgctaaggtggggggtcattgccaggtcatccaacaatataatgttaggacatgtatccgtccaagctgagttaaaacaggtcacagaagtctatgacttggtgcgagaaatgggccctgaatctggacttctacatttaaggcaaagcccaaaagctggactgttatcactagccaactgtcccaactttgcaagtgttgttctcggaaatgcctcaggcttaggcataatcggtatgtatcgagggagagtaccaaacacagaattattttcagcagctgaaagttatgccaaaagtttgaaagaaagcaataaaataaatttctcttcattaggacttacagatgaagagaaagaggctgcagaacatttcttaaatgtgagtgacgacagtcaaaatgattatgagtaattaaaaaagtgggacaagtcaaaatgtcattccctgaaggaaaagatattcttttcatgggtaatgaagcagcaaaattagcagaagctttccagaaatcattaagaaaaccaggtcataaaagatctcaatctattataggagaaaaagtgaatactgtatcagaaacattggaattacctactatcagtagacctgcaaaaccaaccataccgtcagaaccaaagttagcatggacagataaaggtggggcaaccaaaactgaaataaagcaagcaatcaaagtcatggatcccattgaagaagaagagtctaccgagaagaaggtgctaccctccagtgatgggaaaacccctgcagaaaagaaactgaaaccatcaactaacaccaaaaagaaggtttcatttacaccaaatgaaccagggaaatatacaaagttggaaaaagatgctctagatttgctctcagataatgaagaagaagatgcagaatcttcaatcttaacctttgaagaaagagatacttcatcattaagcattgaggccagattggaatcaatagaggagaaattaagcatgatattagggctattaagaacactcaacattgctacagcaggacccacagcagcaagagatgggatcagagatgcaatgattggcgtaagagaggaattaatagcagacataataaaggaagctaaagggaaagcagcagaaatgatggaagaggaaatgagtcaacgatcaaaaataggaaatggtagtgtaaaattaacagaaaaagcaaaagagctcaacaaaattgttgaagatgaaagcacaagtggagaatccgaagaagaagaagaaccaaaagacacacaagacaatagtcaagaagatgacatttaccagttaattatgtagtttaataaaaataaacaatgggacaagtaaaaatggagtcctacctagtagacacctatcaaggcattccttacacagcagctgttcaagttgatctaatagaaaaggacctgttacctgcaagcctaacaatatggttccctttgtttcaggccaacacaccaccagcagtgctgctcgatcagctaaaaaccctgacaataaccactctgtatgctgcatcacaaaatggtccaatactcaaagtgaatgcatcagcccaaggtgcagcaatgtctgtacttcccaaaaaatttgaagtcaatgcgactgtagcactcgatgaatatagcaaactggaatttgacaaactcacagtctgtgaagtaaaaacagtttacttaacaaccatgaaaccatacgggatggtatcaaaatttgtgagctcagccaaatcagttggcaaaaaaacacatgatctaatcgcactatgtgattttatggatctagaaaagaacacacctgttacaataccagcattcatcaaatcagtttcaatcaaagagagtgagtcagctactgttgaagctgctataagcagtgaagcagaccaagctctaacacaggccaaaattgcaccttatgcgggattaattatgatcatgactatgaacaatcccaaaggcatattcaaaaagcttggagctgggactcaagtcatagtagaactaggagcatatgtccaggctgaaagcataagcaaaatatgcaagacttggagccatcaagggacaagatatgtcttgaagtccagataacaaccaagcaccttggccaagagctactaaccctatctcatagatcataaagtcaccattctagttatataaaaatcaagttagaacaagaattaaatcaatcaagaacgggacaaataaaaatgtcttggaaagtggtgatcattttttcattgttaataacacctcaacacggtcttaaagagagctacttagaagagtcatgtagcactataactgaaggatatctcagtgttctgaggacaggttggtacaccaatgtttttacactggaggtaggcgatgtagagaaccttacatgtgccgatggacccagcttaataaaaacagaattagacctgaccaaaagtgcactaagagagctcagaacagtttctgctgatcaactggcaagagaggagcaaattgaaaatcccagacaatctagattcgttctaggagcaatagcactcggtgttgcaactgcagctgcagttacagcaggtgttgcaattgccaaaaccatccggcttgaaagtgaagtaacagcaattaagaatgccctcaaaaagaccaatgaagcagtatctacattggggaatggagttcgtgtgttggcaactgcagtgagagagctgaaagattttgtgagcaagaatctaacacgtgcaatcaacaaaaacaagtgcgacattgctgacctgaaaatggccgttagcttcagtcaattcaacagaaggttcctaaatgttgtgcggcaattttcagacaacgctggaataacaccagcaatatctttggacttaatgacagatgctgaactagccagagctgtttccaacatgccaacatctgcaggacaaataaaactgatgttggagaaccgtgcaatggtaagaagaaaagggttcggattcctgataggagtttacggaagctccgtaatttacatggtgcaactgccaatctttggggttatagacacgccttgctggatagtaaaagcagccccttcttgttcaggaaaaaagggaaactatgcttgcctcttaagagaagaccaaggatggtattgtcaaaatgcagggtcaactgtttactacccaaatgaaaaagactgtgaaacaagaggagaccatgtcttttgcgacacagcagcaggaatcaatgttgctgagcagtcaaaggagtgcaacataaacatatctactactaattacccatgcaaagttagcacaggaagacatcctatcagtatggttgcactatctcctcttggggctttggttgcttgctacaagggagtgagctgttccattggcagcaacagagtagggatcatcaagcaactgaacaaaggctgctcttatataaccaaccaagacgcagacacagtgacaatagacaacactgtataccagctaagcaaagttgaaggcgaacagcatgttataaaaggaaggccagtgtcaagcagctttgacccagtcaagtttcctgaagatcaattcaatgttgcacttgaccaagttttcgagagcattgagaacagtcaggccttggtggatcaatcaaacagaatcctaagcagtgcagagaaaggaaacactggcttcatcattgtaataattctaattgctgtccttggctctaccatgatcctagtgagtgtttttatcataataaagaaaacaaagaaacccacaggagcacctccagagctgagtggtgtcacaaacaatggcttcataccacataattagttaattaaaaataaagtaaattaaaataaattaaaattaaaaataaaaatttgggacaaatcataatgtctcgcaaggctccgtgcaaatatgaagtgcggggcaaatgcaatagaggaagtgagtgcaagtttaaccacaattactggagttggccagatagatacttattaataagatcaaattatttattaaatcaacttttaaggaacactgatagagctgatggcttatcaataatatcaggagcaggcagagaagataggacacaagattttgtcctaggttccaccaatgtggttcaaggttatattgatgataaccaaagcataacaaaagctgcagcctgttacagtctacataatataatcaaacaactacaagaagttgaagttaggcaggctagagataacaaactatctgacagcaaacatgtagcacttcacaacttagtcctatcttatatggagatgagcaaaactcctgcatctttaatcaacaatctcaagagactgccgagagagaaactgaaaaaattagcaaagctcataattgacttatcagcaggtgctgaaaatgactcttcatatgccttgcaagacagtgaaagcactaatcaagtgcagtgagcatggtccagttttcattactatagaggttgatgacatgatatggactcacaaggacttaaaagaagctttatctgatgggatagtgaagtctcatactaacatttacaattgttatttagaaaacatagaaattatatatgtcaaggcttacttaagttagtaaaaacacatcagagtgggataaatgacaatgataacattagatgtcattaaaagtgatgggtcttcaaaaacatgtactcacctcaaaaaaataattaaagaccactctggtaaagtgcttattgtacttaagttaatattagctttactaacatttctcacagtaacaatcaccatcaattatataaaagtggaaaacaatctgcaaatatgccagtcaaaaactgaatcagacaaaaaggactcatcatcaaataccacatcagtcacaaccaagactactctaaatcatgatatcacacagtattttaaaagtttgattcaaaggtatacaaactctgcaataaacagtgacacatgctggaaaataaacagaaatcaatgcacaaatataacaacatacaaatttttatgttttaaatctgaagacacaaaaaccaacaattgtgataaactgacagatttatgcagaaacaaaccaaaaccagcagttggagtgtatcacatagtagaatgccattgtatatacacagttaaatggaagtgctatcattacccaaccgatgaaacccaatcctaaatgttaacaccagattaggatccatccaagtctgttagttcaacaatttagttatttaaaaatattttgaaaacaagtaagtttctatgatacttcataataataagtaataattaattgcttaatcatcatcacaacattattcgaaaccataactattcaatttaaaaagtaaaaaacaataacatgggacaagtagttatggaggtgaaagtggagaacattcgaacaatagatatgctcaaagcaagagtaaaaaatcgtgtggcacgcagcaaatgctttaaaaatgcctctttggtcctcataggaataactacattgagtattgccctcaatatctatctgatcataaactataaaatgcaaaaaaacacatctgaatcagaacatcacaccagctcatcacccatggaatccagcagagaaactccaacggtccccacagacaactcagacaccaactcaagcccacagcatccaactcaacagtccacagaaggctccacactctactttgcagcctcagcaagctcaccagagacagaaccaacatcaacaccagatacaacaaaccgcccgcccttcgtcgacacacacacaacaccaccaagcgcaagcagaacaaagacaagtccggcagtccacacaaaaaacaacccaaggacaagctctagaacacattctccaccacgggcaacgacaaggacggcacgcagaaccaccactctccgcacaagcagcacaagaaagagaccgtccacagcatcagtccaacctgacatcagcgcaacaacccacaaaaacgaagaagcaagtccagcgagcccacaaacatctgcaagcacaacaagaatacaaaggaaaagcgtggaggccaacacatcaacaacatacaaccaaactagttaacaaaaaatacaaaataactctaagataaaccatgcagacaccaacaatggagaagccaaaagacaattcacaatctccccaaaaaggcaacaacaccatattagctctgcccaaatctccctggaaaaaacactcgcccatataccaaaaataccacaaccaccccaagaaaaaaactgggcaaaacaacacccaagagacaaataacaatggatcctctcaatgaatccactgttaatgtctatcttcctgactcatatcttaaaggagtgatttcctttagtgagactaatgcaattggttcatgtctcttaaaaagaccttacctaaaaaatgacaacactgcaaaagttgccatagagaatcctgttatcgagcatgttagactcaaaaatgcagtcaattctaagatgaaaatatcagattacaagatagtagagccagtaaacatgcaacatgaaattatgaagaatgtacacagttgtgagctcacattattaaaacagtttttaacaaggagtaaaaatattagcactctcaaattaaatatgatatgtgattggctgcagttaaagtctacatcagatgatacctcaatcctaagttttatagatgtagaatttatacctagctgggtaagcaattggtttagtaattggtacaatctcaacaagttgattctggaattcaggaaagaagaagtaataagaactggttcaatcttgtgtaggtcattgggtaaattagtttttgttgtatcatcatatggatgtatagtcaagagcaacaaaagcaaaagagtgagcttcttcacatacaatcaactgttaacatggaaagatgtgatgttaagtagattcaatgcaaatttttgtatatgggtaagcaacagtctgaatgaaaatcaagaagggctagggttgagaagtaatctgcaaggcatattaactaataagctatatgaaactgtagattatatgcttagtttatgttgcaatgaaggtttctcacttgtgaaagagttcgaaggctttattatgagtgaaattcttaggattactgaacatgctcaattcagtactagatttagaaatactttattaaatggattaactgatcaattaacaaaattaaaaaataaaaacagactcagagttcatggtaccgtgttagaaaataatgattatccaatgtacgaagttgtacttaagttattaggagatactttgagatgtattaaattattaatcaataaaaacttagagaatgctgctgaattatactatatatttagaatattcggtcacccaatggtagatgaaagagatgcaatggatgctgtcaaattaaacaatgaaatcacaaaaatccttaggtgggagagcttgacagaactaagaggggcattcatattaaggattatcaaaggatttgtagacaacaacaaaagatggcccaaaattaaaaacttaaaagtgcttagtaagagatggactatgtacttcaaagcaaaaagttaccccagtcaacttgaattaagcgaacaagattttttagagcttgctgcaatacagtttgaacaagagttttctgtccctgaaaaaaccaaccttgagatggtattaaatgataaagctatatcacctcctaaaagattaatatggtctgtgtatccaaaaaattacttacctgagaaaataaaaaatcgatatctagaagagactttcaatgcaagtgatagtctcaaaacaagaagagtactagagtactatttgaaagataataaattcgaccaaaaagaacttaaaagttatgttgttaaacaagaatatttaaatgataaggatcatattgtctcgctaactggaaaagaaagagaattaagtgtaggtagaatgtttgctatgcaaccaggaaaacagcgacaaatacaaatattggctgaaaaattgttagctgataatattgtaccttttttcccagaaaccttaacaaagtatggtgatctagatcttcagagaataatggaaatcaaatcggaactttcttctattaaaactagaagaaatgatagttataataattacattgcaagagcatccatagtaacagatttaagtaagttcaaccaagcctttaggtatgaaactacagcgatctgtgcggatgtagcagatgaactacatggaacacaaagcctattctgttggttacatcttatcgtccctatgacaacaatgatatgtgcctatagacatgcaccaccagaaacaaaaggtgaatatgatatagataagatagaagagcaaagtggtttatatagatatcatatgggtggtattgaaggatggtgtcaaaaactctggacaatggaagctatatctctattagatgttgtatctgtaaaaacacgatgtcaaatgacatctttattaaacggtgacaaccaatcaatagatgtaagtaaaccagttaagttatctgagggtttagatgaagtgaaagcagattatagcttggctgtaaaaatgttaaaagaaataagagatgcatacagaaatataggccataaacttaaagaaggggaaacatatatatcaagagatcttcagtttataagtaaggtgattcaatctgaaggagtaatgcatcctacccctataaaaaagatcttaagagtgggaccatggataaacacaatattagatgacattaaaaccagtgcagagtcaatagggagtctatgtcaggaattagaatttaggggggaaagcataatagttagtctgatattaaggaatttttggctgtataatttatacatgcatgaatcaaagcaacaccccctagcagggaagcagttattcaaacaactaaataaaacattaacatcagtgcagagattttttgaaataaaaaaggaaaatgaagtagtagatctatggatgaacataccaatgcagtttggaggaggagatccagtagtcttctatagatctttctatagaaggacccctgattttttaactgaagcaatcagtcatgtggatattctgttaagaatatcagccaacataagaaatgaagcgaaaataagtttcttcaaagccttactgtcaatagaaaaaaatgaacgtgctacactgacaacactaatgagagatcctcaagctgttggctcagagcgacaagcaaaagtaacaagtgatatcaatagaacagcagttaccagcatcttaagtctttctccaaatcaacttttcagcgatagtgctatacactacagtagaaatgaagaagaggtcggaatcattgctgacaacataacacctgtttatcctcatggactgagagttttgtatgaatcattaccttttcataaagctgaaaaagttgtgaatatgatatcaggaacgaaatccataaccaacttattacagagaacatctgctattaatggtgaagatattgacagagctgtatccatgatgctggagaacctaggattattatctagaatattgtcagtagttgttgatagtatagaaattccaaccaaatctaatggtaggctgatatgttgtcagatatctagaaccctaagggagacatcatggaataatatggaaatagttggagtaacatcccctagcatcactacatgcatggatgtcatatatgcaactagctctcatttgaaagggataatcattgaaaagttcagcactgacagaactacaagaggtcaaagaggtccaaagagcccttgggtagggtcgagcactcaagagaaaaaattagttcctgtttataacagacaaattctttcaaaacaacaaagagaacagctagaagcaattggaaaaatgagatgggtatataaagggacaccaggtttaagacgattactcaataagatttgtcttggaagtttaggcattagttacaaatgtgtaaaacctttattacctaggtttatgagtgtaaatttcctacacaggttatctgtcagtagtagacctatggaattcccagcatcagttccagcttatagaacaacaaattaccattttgacactagtcctattaatcaagcactaagtgagagatttgggaatgaagatattaatttggtcttccaaaatgcaatcagctgtggaattagcataatgagtgtagtagaacaattaactggtaggagtccaaaacagttagttttaatacctcaattagaagaaatagacattatgccaccaccagtgtttcaagggaaattcaattataagctagtagataagataacttctgatcaacatatcttcagtccagacaaaatagatatgttaacactggggaaaatgctcatgcccactataaaaggtcagaaaacagatcagttcctgaacaagagagagaattatttccatgggaataatcttattgagtctttgtcagcagcgttagcatgtcattggtgtgggatattaacagagcaatgtatagaaaataatattttcaagaaagactggggtgacgggttcatatcggatcatgcttttatggacttcaaaatattcctatgtgtctttaaaactaaacttttatgtagttgggggtcccaagggaaaaacattaaagatgaagatatagtagatgaatcaatagataaactgttaaggattgataatactttttggagaatgttcagcaaggttatgtttgaatcaaaggttaagaaaaggataatgttatatgatgtaaaatttctatcattagtaggttatatagggtttaagaattggtttatagaacagttgagatcagctgagttgcatgaggtaccttggattgtcaatgccgaaggtgatctggttgagatcaagtcaattaaaatctatttgcaactgatagagcaaagtttatttttaagaataactgttttgaactatacagatatggcacatgctctcacaagattaatcagaaagaagttgatgtgtgataatgcactattaactccgattccatccccaatggttaatttaactcaagttattgatcctacagaacaattagcttatttccctaagataacatttgaaaggctaaaaaattatgacactagttcaaattatgctaaaggaaagctaacaaggaattacatgatactgttgccatggcaacatgttaatagatataactttgtctttagttctactggatgtaaagttagtctaaaaacatgcattggaaaacttatgaaagatctaaaccctaaagttctgtactttattggagaaggggcaggaaattggatggccagaacagcatgtgaatatcctgacatcaaatttgtatacagaagtttaaaagatgaccttgatcatcattatcctttggaataccagagagttataggagaattaagcaggataatagatagcggtgaagggctttcaatggaaacaacagatgcaactcaaaaaactcattgggatttgatacacagagtaagcaaagatgctttattaataactttatgtgatgcagaatttaaggacagagatgatttttttaagatggtaattctatggaggaaacatgtattatcatgcagaatttgcactacttatgggacagacctctatttattcgcaaagtatcatgctaaagactgcaatgtaaaattacctttttttgtgagatcagtagccacctttattatgcaaggtagtaaactgtcaggctcagaatgctacatactcttaacactaggccaccacaacaatttaccctgccatggagaaatacaaaattctaagatgaaaatagcagtgtgtaatgatttttatgctgcaaaaaaacttgacaataaatctattgaagccaactgtaaatcacttttatcagggctaagaataccgataaataagaaagaattaaatagacagagaaggttattaacactacaaagcaaccattcttctgtagcaacagttggaggtagcaaggtcatagagtctaaatggttaacaaacaaggcaaacacaataattgattggttagaacatattttaaattctccaaaaggtgaattaaattatgatttttttgaagcattagaaaatacttaccctaatatgattaaactaatagataatctagggaatgcagagataaaaaaactgatcaaagtaactggatatatgcttgtaagtaaaaaatgaaaaatgataaaaatgataaaataggtgacaacttcatactattccaaagtaatcatttgattatgcaattatgtaatagttaattaaaaactaaaaatcaaaagttagaaactaacaactgtcattaagtttattaaaaataagaaattataattggatgtatacg

SEQ ID NO: 2 - DNA, 782 bp, human metapneumovirus
  gene (1)...(782): human metapneumovirus, strain Quebec Isolate No hMPV-118
atgtaggcaccacaactgcagtgacaccttcatcattgcagcaagagataacacttttgtgtggagagattctttacactaaacatactgattacaaatatgctgcagagatagggatacaatatatttgcacggctctaggatcagaaagagtacaacagattttaagaaattcaggcagtgaagttcaggtggttctaaccaagacatactctttagggaaaggtaaaaatagtaaaggggaagagttgcaaatgttagatatacatggagtggaaaagagttgggtagaagaaatagacaaagaggcaagaaaaacaatggtgactttgctaaaggaatcatcaggtaacatcccacaaaaccagaggccttcagcaccagacacaccaataattttattatgtgtaggtgctttaatattcactaaactagcatcaacaatagaagttggactagagactacagttagaagagctaacagagtgctaagtgatgcgactcaaagataccctagggtagatataccaaagattgctagatctttctatgaactatttgagcagaaagtgtattacaggagtctattcattgagtatgggaaagctttaggctcatcttcgacaggaagcaaagcagaaagtttgtttgtaaatatatttatgccagcttatggagccggtcaaacaatgctaaggtggggtgtcattgccagatcatctaataatacattgctagggcatgtatctgtgcaacctggatttgaagcaagtcacagaggt

SEQ ID NO: 3 - DNA, 878 bp, human metapneumovirus
  gene (1)...(878): human metapneumovirus, strain Quebec Isolate No hMPV-174
atgtgggtaccaccactgcagtgacaccctcatcattgcaacaagaaataacgctgttgtgtggagaaattctgtatgctaaacatgctgattacaaatatgctgcagaaataggaatacaatatattagcacagctttaggatcagagagagtgcagcagattctgaggaactcaggcagtgaagtccaagtggtcttaaccagaacgtactctctggggaaagttaaaaacaataaaggagaagatttacagatgttagacatacacggggtagagaagagctgggtagaagagatagacaaagaagcaaggaaaacaatggcaaccttgcttaaggaatcatcaggtaatatcccgcaaaatcagaggccctcagcaccagacacacccataatcttattatgtgtaggtgccttaatatttactaaactagcatcaaccatagaagtgggactagagaccacagtcagaagggctaaccgtgtactaagtgatgcactcaagagataccctagaatggacatcccaaaaattgccagatccttctatgacttatttgaacaaaaagtgtatcacagaagtttgttcattgagtatggcaaagcattaggctcatcatctacaggcagcaaagcagaaagtctatttgttaatatattcatgcaagcttatggagccggtcaaacaatgctaaggtggggggtcattgccaggtcatccaacaatataatgttatgacatgtatccgtccaagctgagttaaacacaggtcacagaagtctatgacttggtgcgagaaatgggtcctgaatctggacttctacatttaaggcaaagcccaaaagctggactgtatcactagccaacttcccaact

SEQ ID NO: 4 - DNA, 876 bp, human metapneumovirus
  gene (1)...(876): human metapneumovirus, strain Quebec Isolate No hMPV-176
atgtaggccccacaactgcagtgacaccttcatcattgcagcaagagataacacttttgtgtggagagattctttacactaaacatactgattacaaatatgctgcagagatagggatacaatatatttgcacggctctaggatcagaaagagtacaacagattttaagaaattcaggtagtgaagttcaggtggttctaaccaagacatactctttagggaaaggtaaaaatagtaaaggggaagagttgcaaatgttagatatacatggagtggaaaagagttgggtagaagaaatagacaaagaggcaagaaagacaatggtgactttgctaaaggaatcatcaggtaacatcccacaaaaccagaggccttcagcaccagacacaccaataattttattatgtgtaggtgcgttaatattcactaaactagcatcaacaatagaagttggactagagactacagttagaagagctaacagagtgctaagtgatgcgctcaaaagataccctagggtagatataccaaagattgctagatctttttatgaactatttgagcagaaagtgtattacaggagtctattcattgagtatgggaaagctttaggctcatcttcaacaggaagcaaagcagaaagtttgtttgtaaatatatttatgcaagcttatggagccggtcaaacaatgctaaggtggggtgtcattgccagatcatctaacaacataatgctagggcatgtatctgtgcaagctgaattgaaacaagttacagaggtttatgatttggtgagagaaatgggtcctgaatctgggcttttactcttaagaacaagtccaaaggcaggactgttatcgtggctaatgccccaat

SEQ ID NO: 5 - DNA, 869 bp, human metapneumovirus
  gene (1)...(869): human metapneumovirus, strain Qubec Isolate No hMPV-193
atgtgggtacaacaactgcagtgacaccctcatcattgcaacaagaaataacgctgttgtgtggagaaattctgtatgctaaacatgctgattacaaatatgctgcagaaataggaatacaatatattagcacagctttaggatcagagagagtgcagcagattctgaggaactcaggtagtgaagtccaagtggtcttaaccagaacgtactctctggggaaagttaaaaacaataaaggagaagatttacagatgttagacatacacggggtagagaagagctgggtagaagagatagacaaagaagcaaggaaaacaatggcaaccttgcttaaggaatcatcaggtaatatcccacaaaatcagaggccctcagcaccagacacacccataatcttattatgtgtaggtgccttaatatttactaagctagcatcaaccatagaagtgggactagagaccacagtcagaagggctaaccgtgtactaagtgatgcactcaagagataccctagaatggacatcccaaaaattgccagatccttctatgacttatttgaacaaaaagtgtatcacagaagtttgttcattgagtatggtaaagcattaggatcatcatctacaggcagcaaagcagaaagtctatttgttaatatattcatgcaagcttatggagccggtcaaacaatgctaaggtggggggtcattgccaggtcatccaacaatataatgttaggacatgtatccgtccaaggctgagttaaaacaggtcacagaagtctatgacttggtgcgagaaatgggtcctgaatctggacttctacatttaaggcaaagcccaaaagctggactgttatcactagccaa

SEQ ID NO: 6 - DNA, 848 bp, human metapneumovirus
  gene (1)...(848): human metapneumovirus, strain Quebec Isolate No C-77807
atgtaggcaccacaactgcagtgacaccttcatcattgcaacaagaaataacacttttgtgtggggaaattctttacactaaacacactgattacaaatatgctgctgagataggaatgcaatatatttgcacagctctaggatcagaaagagtacaacagattttgagaaactcaggtagtgaagttcaggtggttttaaccaaaacatactccttagggaaaggcaaaaacagtaaaggggaagagctgcagatgttagatatacatggagtggaaaagagttgggtagaagaaatagacaaagaggcaagaaagacaatggtaactttgcttaaggagtcatcaggtaacatcccacaaaaccagagaccttcagcaccagacacaccaataattttattatgtgtaggtgccctaatattcactaaactagcatcaacaatagaagttggattagagactacagttagaagagctaatagagtgctaagtgatgcactcaaaagataccctagggtagatataccaaagattgctagatctttttatgaactatttgaacaaaaagtgtactacagaagtttattcattgagtacggaaaagctttaggctcatcttcaacaggaagcaaagcagaaagtttgtttgtaaatatatttatgcaagcttatggagctggtcaaacactgctaaggtggggtgtcattgccagatcatccaacaacataatgctagggcatgtatctgtgcaatctgaattgaagcaattcacagaggtttatgacttggtgagagaaatgggtcctgaatctgggcttttacatctaagacaaagtccaaaggca

SEQ ID NO: 7 - DNA, 894 bp, human metapneumovirus
  gene (1)...(894): human metapneumovirus, strain Quebec Isolate No C-77748
atgtgggtacaacaactgcagtgacaccctcatcattgcaacaagaaataacgctgttgtgtggagaaattctgtatgctaaacatgctgattacaaatatgctgcagaaataggaatacaatatattagcacagctttaggatcagagagagtgcagcagattctgaggaactcaggcagtgaagtccaagtggtcttaaccagaacgtactctctggggaaagttaaaaacaataaaggagaagatttacagatgttagacatacacggggtagagaagagctgggtagaagagatagacaaagaagcaaggaaaacaatggcaaccttgcttaaggaatcatcaggtaatatcccacaaaatcagaggccctcagcaccagacacacccataatcttattatgtgtaggtgccttaatatttactaagctagcatcaaccatagaagtgggactagagaccacagtcagaagggctaaccgtgtactaagtgatgcacctaagagataccctagaatggacatcccaaaaattgccagatccttctatgacttatttgaacaaaaagtgtatcacagaagtttgttcattgagtatggcaaagcattaggctcatcatctacaggcagcaaacgagaaagtctatttgcttatatattcatgcgagcttatggagccggtccaacaatgctaaggtggggggtcattgccagatcatccaacaatataatgttaggacatgtatctgtccaagctgaatttaaacaagacacagaagtccatgacttggtggtagaaatggggcctgaatctggactttctactttaaggcaaagcccaaaagctggactgtttacactagccaactgtcccaactttgcaagtgttgttc

SEQ ID NO: 8 - DNA, 850 bp, human metapneumovirus
  gene (1)...(850): human metapneumovirus, strain Quebec Isolate No hMPV-217
atgtgggtacaacaactgcagtgacaccctcatcattgcaacaagaaataacgctgttgtgtggagaaattctgtatgctaaacatgctgattacaaatatgctgcagaaataggaatacaatatattagcacagctttaggatcagagagagtgcagcagattctgaggaactcaggtagtgaagtccaagtggtcttaaccagaacgtactctctggggaaagttaaaaacaataaaggagaagatttacagatgttagacatacacggggtagagaagagctgggtagaagagatagacaaagaagcaaggaaaacaatggcaaccttgcttaaggaatcatcaggtaatatcccacaaaatcagaggccctcagcaccagacacacccataatcttattatgtgtaggtgccttaatatttactaagctagcatcaaccatagaagtgggactagagaccacagtcagaagggctaaccgtgtactaagtgatgcactcaagagataccctagaatggacatcccaaaaattgccagatccttctatgacttatttgaacaaaaagtgtatcacagaagtttgttcattgagtatggtaaagcattaggatcatcatctacaggcagcaaagcagaaagtctatttgttaatatattcatgcaagcttatggagccggtcaaacaatgctaaggtggggggtcattgccaagtcatccaacaatataatgttaggacatgtatccgtccaagctgaattaaaaacaggtcccagaagtccatgacttggtgcgagaaatggggcctgaatctggacttctacttttaaggcaaagcccaaaagctg

SEQ ID NO: 9 - DNA, 1185 bp, human metapneumovirus
  gene (1)...(1185): human metapneumovirus, strain 00-1, accession number af371337
atgtctcttcaagggattcacctgagtgatttatcatacaagcatgctatattaaaagagtctcagtacacaataaaaagagatgtgggtacaacaactgcagtgacaccctcatcattgcaacaagaaataacactgttgtgtggagaaattctgtatgctaaacatgctgactacaaatatgctgcagaaataggaatacaatatattagcacagctttaggatcagagagagtgcagcagattctgaggaactcaggcagtgaagtccaagtggtcttaaccagaacgtactctctggggaaaattaaaaacaataaaggagaagatttacagatgttagacatacacggggtagagaagagctgggtagaagagatagacaaagaagcaaggaaaacaatggcaaccttgcttaaggaatcatcaggtaatatcccacaaaatcagaggccctcagcaccagacacacccataatcttattatgtgtaggtgccttaatattcactaaactagcatcaaccatagaagtgggactagagaccacagtcagaagggctaaccgtgtactaagtgatgcactcaagagataccctagaatggacataccaaagattgccagatccttctatgacttatttgaacaaaaagtgtatcacagaagtttgttcattgagtatggcaaagcattaggctcatcatctacaggcagcaaagcagaaagtctatttgttaatatattcatgcaagcttatggggccggtcaaacaatgctaaggtggggggtcattgccaggtcatccaacaatataatgttaggacatgtatccgtccaagctgagttaaaacaggtcacagaagtctatgacttggtgcgagaaatgggccctgaatctggacttctacatttaaggcaaagcccaaaagctggactgttatcactagccaactgtcccaactttgcaagtgttgttctcggaaatgcctcaggcttaggcataatcggtatgtatcgagggagagtaccaaacacagaattattttcagcagctgaaagttatgccaaaagtttgaaagaaagcaataaaataaatttctcttcattaggacttacagatgaagagaaagaggctgcagaacatttcttaaatgtgagtgacgacagtcaaaatgattatgagtaa

SEQ ID NO: 10 - DNA, 700 bp, human metapneumovirus
  gene (1)...(700): human metapneumovirus, strain Quebec Isolate No hMPV-163
tgcagaaataggaatacaatatattagcacagctttaggatcagagagagtgcagcagattctgaggaactcaggcagtgaagtccaagtggtcttaaccagaacgtactctctggggaaagttaaaaacaataaaggagaagatttacagatgttagacatacacggggtagagaagagctgggtagaagagatagacaaagaagcaaggaaaacaatggcaaccttgcttaaggaatcatcaggtaatatcccacaaaatcagaggccctcagcaccagacacacccataatcttattatgtgtaggtgccttaatatttactaaactagcatcaaccatagaagtgggactagagaccacagtcagaagggccaaccgtgtactaagtgatgcactcaagagataccctagaatggacatcccaaaaattgccagatccttctatgacttatttgaacaaaaagtgtatcacagaagtttgttcattgagtatggcaaagcattaggctcatcatctacaggcagcaaagcagaaagtctatttgttaatatattcatgcaagcttatggagccggtcaaacaatgctaaggttgggggtaattgccaggtcatccaacaatataatgttaggacatgtatccgtccaagctgagttaaaacaggtcacagaagtctatgacttggtgcgagaaatg

SEQ ID NO: 11 - DNA, 705 bp, human metapneumovirus
  CDS (1)...(705): human metapneumovirus, strain Quebec Isolate No hMPV-174
tattagcacagctttaggatcagagagagtgcagcagattctgaggaactcaggcagtgaagtccaagtggtcttaaccagaacgtactctctggggaaagttaaaaacaataaaggagaagatttacagatgttagacatacacggggtagagaagagctgggtagaagagatagacaaagaagcaaggaaaacaatggcaaccttgcttaaggaatcatcaggtaatatcccgcaaaatcagaggccctcagcaccagacacacccataatcttattatgtgtaggtgccttaatatttactaaactagcatcaaccatagaagtgggactagagaccacagtcagaagggctaaccgtgtactaagtgatgcactcaagagataccctagaatggacatcccaaaaattgccagatccttctatgacttatttgaacaaaaagtgtatcacagaagtttgttcattgagtatggcaaagcattaggctcatcatctacaggcagcaaagcagaaagtgtatttgttaatatattcatgcaagcttatggagccggtcaaacaatgctgaggtggggggtcattgccaggtcatccaacaatataatgttaggacatgtatccgtcctagctgagctaaaaccggtcacagaagtctatgacttggtgcgagaaatgattcctgaatctngacttctacatta

SEQ ID NO: 12 - DNA, 700 bp, human metapneumovirus
  gene (1)...(700): human metapneumovirus, strain Quebec Isolate No hMPV-184
aaatatgctgcagaaataggaatacaatatattagcacagctttaggatcagagagagtgcagcagattctgaggaactcaggcagtgaagtccaagtggtcttaaccagaacgtactctctggggaaagttaaaaacaataaaggagaagatttacagatgttagacatacacggggtagagaagagctgggtagaagagatagacaaagaagcaaggaaaacaatggcaaccttgcttaaggaatcatcaggtaatatcccacaaaatcagaggccctcagcaccagacacacccataatcttattatgtgtaggtgccttaatatttactaaactagcatcaaccatagaagtgggactagagaccacagtcagaagggccaaccgtgtactaagtgatgcactcaagagataccctagaatggacatcccaaaaattgccagatccttctatgacttatttgaacaaaaagtgtatcacagaagtttgttcattgagtatggcaaagcattaggctcatcatctacaggcagcaaagcagaaagtctatttgttaatatattcatgcaagcttatggagccggtcaaacaatgctaaggtggggggtaattgccagatcatccaacaatataatgttaggacatgtatccgtccaagctgagttaaaacaggtcacagaagtctatgacttagtgc

SEQ ID NO: 13 - DNA, 650 bp, human metapneumovirus
  gene (1)...(650): human metapneumovirus, strain Quebec Isolate No hMPV-185
taggaatacaatatattagcacagctctaggatcagagagagtacagcagattctaagaaactcaggcagtgaagtccaagtggttttaaccagaacgtactccttggggaaagttaaaaacaacaaaggagaagatttacagatgttagacatacacggagtagagaaaagctgggtggaagagatagacaaagaagcaagaaaaacaatggcaactttgcttaaagaatcatcaggcaatattccacaaaatcagaggccttcagcaccagacacacctataatcttattatgtgtaggtgccttaatatttaccaaactagcatcaactatagaagtgggattagagaccacagtcagaagagctaaccgtgtactaagtgatgcactcaaaagataccctaggatggacataccaaaaatcgctagatctttctatgacttatttgaacaaaaagtgtattacagaagcttgttcattgagtatggcaaagcattaggctcatcctctacaggcagcaaagcagaaagtttattcgttaatatattcatgcaagcttacggtgctggtcaaacaatgctgaggtggggagtcattgccaggtcatctaacaatataatgttaggacatgtatctgtccaagctgag

SEQ ID NO: 14 - DNA, 650 bp, human metapneumovirus
  gene (1)...(650): human metapneumovirus, strain Quebec Isolate No C-76837
acaaatatgcatcataaataggaatacaatatattagcacagctttagagatcagagagagtgcagcagattctaaggaactcaggcagtgaagtccaagtggtcttaaccagaacgtactctctggggaaagttaaaaacaataaaggagaagatttacagatgttagacatacacggggtagagaagagctgggtagaagagatagacaaagaagcaaggaaaacaatggcaaccttgcttaaggaatcatcaggtaatatcccacaaaatcagaggccctcagcaccagacacacccataatcttattatgtgtaggtgccttaatatttactaaactagcatcaaccatagaagtgggactagagaccacagtcagaagggctaaccgtgtactaagtgatgcactcaagagataccctagaatggacatcccaaaaattgccagatccttctatgacttatttgaacaaaaagtgtatcacagaagtttgttcattgagtatggcaaagcattaggctcatcatctacaggcagcaaagcagaaagtctatttgttaatatattcatgcaagcttatggagccggtcaaacaatgctaaggtggggggtcattgccaggtcatccaacaatataatgttaggata

SEQ ID NO: 15 - DNA, 650 bp, human metapneumovirus
  gene (1)...(650): human metapneumovirus, strain Quebec Isolate No C-77807
gtatgctaaacatgctgattacaaatatgctgcagaaataggaatacaatatattagcacagctttaggatcagagagagtgcagcagattctgaggaactcaggcagtgaagtccaagtggtcttaaccagaacgtactctctggggaaagttaaaaacaataaaaggagaagatttacagatgttagacatacacggggtagagaagagctgggtagaagagatagacaaagaagcaaggaaaacaatggcaaccttgcttaaggaatcatcaggtaatatcccacaaaatcagaggccctcagcaccagacacacccataatcttattatgtgtaggtgccttaatatttactaagctagcatcaaccatagaagtgggactagagaccacagtcagaagggctaaccgtgtactaagtgatgcactcaagagataccctagaatggacatcccaaaaattgccagatccttctatgacttatttgaacaaaaagtgtatcacagaagtttgttcattgagtatggcaaagcattaggctcatcatctacaggcagcaaagcagaaagtctatttgttaatatattcatgcaagcttatggagccggtcaaacaatgctaaggtggggggtcattgccaggtcatcc

SEQ ID NO: 16 - DNA, 650 bp, human metapneumovirus
  gene (1)...(650): human metapneumovirus, strain Quebec Isolate No hMPV-C-78123
ttaggatcagagagagtgcagcagattctaaggaactcaggcagtgaagtccaagtggtcttaaccagaacgtactctctggggaaagttaaaaacaataaaggagaagatttacagatgttagacatacacggggtagagaagagctgggtagaagagatagacaaagaagcaaggaaaacaatggcaaccttgcttaaggaatcatcaggtaatatcccacaaaatcagaggccctcagcaccagacacacccataatcttattatgtgtaggtgccttaatatttactaaactagcatcaaccatagaagtgggactagagaccacagtcagaagggctaaccgtgtactaagtgatgcactcaagagataccctagaatggacatcccaaaaattgccagatccttctatgacttatttgaacaaaaagtgtatcacagaagtttgttcattgagtatggcaaagcattaggctcatcatctacaggcagcaaagcagaaagtctatttgttaatatattcatgcaagcttatggagccggtcaaacaatgctaaggtggggggtcattgccaggtcatccaacaatataatgttaggacatgtatccgtccaagctgagttaaaacaggtcacagaagtctatga

SEQ ID NO: 17 - DNA, 650 bp, human metapneumovirus
  gene (1)...(700): human metapneumovirus, strain Quebec Isolate No C-78132
cagaaagagtacaacagattttgagaaactcaggtagtgaagttcaggtggttttaaccaaaacatactccttagggaaaggcaaaaacagtaaaggggaagagctgcagatgttagatatacatggagtggaaaagagttgggtagaagaaatagacaaagaggcaagaaagacaatggtaactttgcttaaggagtcatcaggtaacatcccacaaaaccagagaccttcagcaccagacacaccaataattttattatgtgtaggtgccctaatattcactaaactagcatctacaatagaagttggattagagactacagttagaagagctaatagagtgctaagtgatgcactcaaaagataccctagggtagatataccaaagattgctagatctttttatgaactatttgaacaaaaagtgtactacagaagtttattcattgagtacggaaaagctttaggctcatcttcaacaggaagcaaagcagaaagtttgtttgtaaatatatttatgcaagcttatggagctggtcaaacactgctaaggtggggtgtcattgccagatcatccaacaacataatgctagggcatgtatctgtgcaatctgaattgaagcaagttacagaggtttatgacttggtg

SEQ ID NO: 18 - DNA, 650 bp, human metapneumovirus
  gene (1)...(650): human metapneumovirus, strain Quebec Isolate No C-78163
ggatcagaaagagtacaacagattttgagaaactcaggtagtgaagttcaggtggttttaaccaaaacatactccttagggaaaggcaaaaacagtaaaggggaagagctgcagatgttagatatacatggagtggaaaagagttgggtagaagaaatagacaaagaggcaagaaagacaatggtaactttgcttaaggagtcatcaggtaacatcccacaaaaccagagaccttcagcaccagacacaccaataattttattatgtgtaggtgccctaatattcactaaactagcatcaacaatagaagttggattagagactacagttagaagagctaatagagtgctaagtgatgcactcaaaagataccctagggtagatataccaaagattgctagatctttttatgaactatttgaacaaaaagtgtactacagaagtttattcattgagtacggaaaagctttaggctcatcttcaacaggaagcaaagcagaaagtttgtttgtaaatatatttatgcaagcttatggagctggtcaaacactgctaaggtggggtgtcattgccagatcatccaacaacataatgctagggcatgtatctgtgcaatctgaattgaagcaagttacagaggtttatgactt

SEQ ID NO: 19 - DNA, 600 bp, human metapneumovirus
  gene (1)...(600): human metapneumovirus, strain Quebec Isolate No C-91205
atattagcacagctctagggatcagagagagtacagcagattctaagaaactcaggcagtgaagtccaagtggttttaaccagaacgtactccttggggaaagttaaaaacaacaaaggagaagatttacagatgttagacatacacggagtagagaaaagctgggtggaagagatagacaaagaagcaagaaaaacaatggcaactttgcttaaagaatcatcaggcaatattccacaaaatcagaggccttcagcaccagacacacctataatcttattatgtgtaggtgccttaatatttaccaaactagcatcaactatagaagtgggattagagaccacagtcagaagagctaaccgtgtactaagtgatgcactcaaaagataccctaggatggacataccaaaaatcgctagatctttctatgacttatttgaacaaaaagtgtattacagaagcttgttcattgagtatggcaaagcattaggctcatcctctacaggcagcaaagcagaaagtttattcgttaatatattcatgcaagcttacggtgctggtcaaacaatgctgaggtggggagtcattgccaggtcatct

SEQ ID NO: 20 - DNA, 700 bp, human metapneumovirus
  gene (1)...(700): human metapneumovirus, strain Quebec Isolate No C-91364
gattacaaatatgctgctgagataggaatgcaatatatttgcacagctctaggatcagaaagagtacaacagattttgagaaactcaggtagtgaagttcaggtggttctaaccaaaacatactccttagggaaaggcaaaaacagtaaaggggaagagctgcagatgttagatatacatggagtggaaaagagttgggtagaagaaatagacaaagaggcaagaaagacaatggtaactttgcttaaggagtcatcaggtaacatcccacaaaaccagagaccttcagcaccagacacaccaataattttattatgtgtaggtgccctaatattcactaaactagcatcaacaatagaagttggattagagactacagttagaagagctaatagagtgctaagtgatgcactcaaaagataccctagggtagatataccaaagattgctagatctttttatgaactatttgaacaaaaagtgtactacagaagtttattcattgagtacggaaaagctttaggctcatcttcaacaggaagcaaagcagaaagtttgtttgtaaatatatttatgcaagcttatggagctggtcaaacactgctaaggtggggtgtcattgccagatcatccaacaacataatgctagggcatgtatctgtgcaatctgaattgaagcaagttacagaggtttatgact

SEQ ID NO: 21 - DNA, 600 bp, human metapneumovirus
  gene (1)...(600): human metapneumovirus, strain quebec Isolate No C-91830
cagctctaggatcagaaagagtacaacagattttgagaaactcaggtagtgaagttcaggtggttctaaccaaaacatactccttagggaaaggcaaaaacagtaaaggggaagagctgcagatgttagatatacatggagtggaaaagagttgggtagaagaaatagacaaagaggcaagaaagacaatggtaactttgcttaaggagtcatcaggtaacatcccacaaaaccagagaccttcagcaccagacacaccaataattttattatgtgtaggtgccttaatattcactaaactagcatcaacaatagaagttggattagagactacagttagaagagctaatagagtgctaagtgatgcactcaaaagataccctagggtagatataccaaagattgctagatctttttatgaactatttgaacaaaaagtgtactacagaagcttattcattgagtacggaaaagctttaggctcatcttcaacaggaagcaaagcagagagtttgtttgtaaatatatttatgcaagcttatggagctggtcaaacactgctaaggtggggtgtcattgccagatcatccaacaacataa

SEQ ID NO: 22 - DNA, 650 bp, human metapneumovirus
  gene (1)...(650): human metapneumovirus, strain Quebec Isolate No hMPV-Q86
atgctgattacaaatatgctgcagaaataggaatacaatatattagcacagctctagggtcagagaaagtacagcagattctaagaaactcaggcagtgaagtccaagcggttttaaccagaacgtactctttggggaaagttaaaaacaataaaggagaagatttacagatgttagacatacacggagtagaaaaaagctgggtggaagagatagacaaagaagcaagaaaaacaatggcaactttacttaaagaatcatcaggtaatattccacaaaatcagaggccttcagcaccagacacacctataatcttattatgtgtaggtgccttaatattcaccaaactagcatcaactatagaagtgggattagagaccacagtcagaagagctaaccgtgtactaagtgatgcactcaaaagataccctagaatggacataccaaaaattgctagatccttctatgacttatttgaacaaaaagtgtatcacagaagtttgttcattgagtatgggcaaagcattaggctcatcctctacaggcagcaaagcagaaagtttattcgtcaatatattcatgcaagcttatggtgctggtcaaacaatgctgaggtgggggggttatcgccaggtcatctaacaatataa

SEQ ID NO: 23 - DNA, 650 bp, human metapneumovirus
  gene (1)...(650): human metapneumovirus, strain Quebec Isolate No hMPV-Q97
tagcacagctctaggatcagagagagtacagcagattctaagaaactcaggcagtgaagtccaagtggttttaaccagaacgtactccttggggaaagttaaaaacaacaaaggagaagatttacagatgttagacatacacggagtagagaaaagctgggtggaagagatagacaaagaagcaagaaaaacaatggcaactttgcttaaagaatcatcaggcaatattccacaaaatcagaggccttcagcaccagacacacctataatcttattatgtgtaggtgccttaatatttaccaaactagcatcaactatagaagtgggattagagaccacagtcagaagagctaaccgtgtactaagtgatgcactcaaaagataccctaggatggacataccaaaaatcgctagatctttctatgacttatttgaacaaaaagtgtattacagaagcttgttcattgagtatggcaaagcattaggctcatcctctacaggcagcaaagcagaaagttttattcgttaatatattcatgcaagcttacggtgctggtcaaacaatgctgaggtggggagtcattgccaggtcatctaacaatataatgttaggacatgtatctgtccaagctgagttaaaacaagtcacg

SEQ ID NO: 24 - DNA, 741 bp, human metapneumovirus
  gene (1)...(741): human metapneumovirus, strain Can 97-83
agtgacaccctcatcattgcaacaagaaataacactattgtgtggagaaattctatatgctaagcatgctgattacaaatatgctgcagaaataggaatacaatatattagcacagctctaggatcagagagagtacagcagattctaagaaactcaggcagtgaagtccaagtggttttaaccagaacgtactccttggggaaagttaaaaacaacaaaggagaagatttacagatgttagacatacacggagtagagaaaagctgggtggaagagatagacaaagaagcaagaaaaacaatggcaactttgcttaaagaatcatcaggcaatattccacaaaatcagaggccttcagcaccagacacacctataatcttattatgtgtaggtgccttaatatttaccaaactagcatcaactatagaagtgggattagagaccacagtcagaagagctaaccgtgtactaagtgatgcactcaaaagataccctaggatggacataccaaaaatcgctagatctttctatgatttatttgaacaaaaagtgtattacagaagtttgttcattgagtatggcaaagcattaggctcatcctctacaggcagcaaagcagaaagtttattcgttaatatattcatgcaagcttacggtgctggtcaaacaatgctgaggtggggagtcattgccaggtcatctaacaatataatgttaggacatgtatctgtccaagctga

SEQ ID NO: 25 - DNA, 650 bp, human metapneumovirus
  gene (1)...(650): human metapneumovirus, strain Can 98-75
caatatatttgcacggctctaggatcagaaagagtacaacagattttaagaaattcaggcagtgaagttcaggtggttctaaccaagacatactctttagggaaaggtaaaaatagtaaaggggaagagttgcaaatgttagatatacatggagtggaaaagagttgggtagaagaaatagacaaagaggcaagaaaaacaatggtgactttgctaaaggaatcatcaggtaacatcccacaaaaccagaggccttcagcaccagacacaccaataattttattatgtgtaggtgctttaatattcactaaactagcatcaacaatagaagttggactagagactacagttagaagagctaacagagtgctaagtgatgcgctcaaaagataccctagggtagatataccgaagattgctagatctttctatgaactatttgagcagaaagtgtattacaggagtctattcattgagtatgggaaagctttaggctcatcttcaacaggaagcaaagcagaaagtttgtttgtaaatatatttatgcaagcttatggagccggtcaaacaatgctaaggtggggtgtcattgccagatcatctaacaacataatgctaggacatgtgtctgtgcaagctgaattgaagca

SEQ ID NO: 26 - DNA, 759 bp, human metapneumovirus
  gene (1)...(759): human metapneumovirus, strain Can 98-75
atgtcttggaaagtggtgattatcatttcgttactcataacacctcagcacggactaaaagaaagttatttagaagaatcatgtagtactataactgaaggatatctcagtgttttaagaacaggttggtacaccaatgtctttacattagaagttggtgatgttgaaaatcttacatgtactgatggacctagcttaatcaaaacagaacttgacctaaccaaaagtgctctaagagaactcaaaacagtttctgctgatcagttagcgagagaagaacaaattgaaaatcccagacaatcaaggtttgtcctaggtgcaatagctcttggtgttgccacagcagcagcagtcacagcaggcattgcgatagccaaaaccataaggcttgagagtgaagtgaatgcaatcaaaggtgctctcaaaacaaccaacgaggcagtatccacactaggaaatggagtgcgagtcctagccaccgcagtaagagagctgaaagaatttgtgagcaaaaacctgactagtgcaattaacaagaacaaatgtgacattgctgacctgaagatggctgtcagcttcagtcaattcaacagaagattcctaaatgttgtgcggcagttttcagacaatgcagggataacaccagcaatatcattggacctaatgactgatgctgagctggccagagctgtatcatacatgccaacatctgcaggacagataaaactaatgttagagaaccgtgcaatggtaagaaga

SEQ ID NO: 27 - DNA, 759 bp, human metapneumovirus
  gene (1)...(759): human metapneumovirus, strain Quebec Isolate No C-67345
atgtcttggaaagtggtgatcattttttcattgctaataacacctcaacacggtcttaaagagagctacctagaagaatcatgtatcactataactgagggatatcttagtgttctgagaacaggttggtataccaacgtttttacattagaggtgggtgatgtagaaaaccttacatgttctgatggacctagcctaataaaaacagaattagatctgaccaaaagtgcactaagagagctcaaaacagtctctgctgaccaattggcaagagaggaacaaattgagaatcccagacaatctaggtttgttctgggagcaatagcactcggtgttgcaacagcagctgcagtcacagcaggtgttgcaattgccaaaaccatccggcttgagagtgaagtcacagcaattaagaatgccctcaaaacgaccaatgaagcagtatctacattggggaatggagttcgagtgttggcaactgcagtgagagagctgaaagactttgtgagcaagaatttaactcgtgcaatcaacaaaaacaagtgcgacattgatgacctaaaaatggccgttagcttcagtcaattcaacagaaggtttctaaatgttgtgcggcaattttcagacaatgctggaataacaccagcaatatctttggacttaatgacagatgctgaactagccagggccgtttccaacatgccgacatctgcaggacaaataaaattgatgttggagaaccgtgcgatggtaagaaga

SEQ ID NO: 28 - DNA, 759 bp, human metapneumovirus
  gene (1)...(759): human metapneumovirus, strain Can 97-83
ataaaaatgtcttggaaagtggtgatcattttttcattgctaataacacctcaacacggtcttaaagagagctacctagaagaatcatgtagcactataactgagggatatcttagtgttctgaggacaggttggtataccaacgtttttacattagaggtgggtgatgtagaaaaccttacatgttctgatggacctagcctaataaaaacagaattagatctgaccaaaagtgcactaagagagctcaaaacagtctctgctgaccaattggcaagagaggaacaaattgagaatcccagacaatctaggtttgttctaggagcaatagcactcggtgttgcaacagcagctgcagtcacagcaggtgttgcaattgccaaaaccatccggcttgagagtgaagtcacagcaattaagaatgccctcaaaacgaccaatgaagcagtatctacattggggaatggagttcgagtgttggcaactgcagtgagagagctgaaagactttgtgagcaagaatttaactcgtgcaatcaacaaaaacaagtgcgacattgatgacctaaaaatggccgttagcttcagtcaattcaacagaaggtttctaaatgttgtgcggcaattttcagacaatgctggaataacaccagcaatatctttggacttaatgacagatgctgaactagccagggccgtttctaacatgccgacatctgcaggacaaataaaattgatgttggagaaccgtgcgatggtg

SEQ ID NO: 29 - DNA, 759 bp, human metapneumovirus
  gene (1)...(759): human metapneumovirus, strain quebec Isolate No C-68661
atgtcttggaaagtggtgatcattttttcattgttaataacacctcaacacggtcttaaagagagctatttagaagagtcatgtagcactataactgaaggatatctcagtgttctgaggacaggttggtataccaacgtttttacactggaggtaggtgatgtagagaaccttacatgtgctgatggacctagcttaataaaaacagaattagacctgaccaaaagtgcactaagagagctcagaacagtttctgctgatcaactggcaagagaggagcaaattgagaatcccagacaatctagatttgttctaggagcaatagcactcggtgttgcaacagcagctgcagttacagcaggtgttgcaattgccaaaaccatccggcttgaaagtgaagtaacagcaattaagaatgccctcaaaaagaccaatgaagcagtatctacattggggaatggagttcgagtgttggcaactgcagtgagggagctgaaagattttgtgagcaagaatctaacacgtgcaatcaacaaaaacaagtgcgacattgctgacctgaaaatggccgttagcttcagtcaattcaacagaaggtttctaaatgttgtgcggcaattttcagacaatgctggaataacaccagcaatatccttggacttaatgacagatgctgaactagccagagctgtttccaacatgccaacatctgcaggacaaataaaactgatgttggagaaccgtgcaatggtaagaaga

SEQ ID NO: 30 - DNA, 759 bp, human metapneumovirus
  gene (1)...(759): human metapneumovirus, strain Quebec Isolate No C-73750
atgtcttggaaagtggtgatcattttttcattgttaataacacctcaacacggtcttaaagagagctatttagaagagtcatgtagcactataactgaaggatatctcagtgttctgaggacaggttggtataccaacgtttttacactggaggtaggtgatgtagagaaccttacatgtgctgatggacctagcttaataaaaacagaattagacctgaccaaaagtgcactaagagagctcagaacagtttctgctgatcaactggcaagagaggagcaaattgagaatcccagacaatctagatttgttctaggagcaatagcactcggtgttgcaacagcagctgcagttacagcaggtgttgcaattgccaaaaccatccggcttgaaagtgaagtaacagcaattaagaatgccctcaaaaagaccaatgaagcagtatctacattggggaatggagttcgagtgttggcaactgcagtgagggaactgaaagattttgtgagcaagaatctaacacgtgcaatcaacaaaaacaagtgcgacattgctgacctgaaaatggccgttagcttcagtcaattcaacagaaaggttctaaatgttgtgcggcaattttcagacaatgctggaataacaccagcaatatccttggacttaatgacagatgctgaactagccagagctgtttccaacatgccaacatctgcaggacaaataaaactgatgttggagaaccgtgcaatggtaagaaga

SEQ ID NO: 31 - DNA, 759 bp, human metapneumovirus
  gene (1)...(759): human metapneumovirus, strain Quebec Isolate No C-74549
atgtcttggaaagtggtgatcattttttcattgctaataacacctcaacacggtcttaaagagagctacctagaagaatcatgtagcactataactgagggatatcttagtgttctgaggacaggttggtataccaacgtttttacattagaggtgggtgatgtagaaaaccttacatgttctgatggacctagcctaataaaaacagaattagatctgaccaaaagtgcactaagagagctcaaaacagtctctgctgaccaattggcaagagaggaacaaattgagaatcccagacaatctaggtttgttctaggagcaatagcactcggtgttgcaacagcagctgcagtcacagcaggtgttgcaattgccaaaaccatccggcttgagagtgaagtcacagcaattaagaatgccctcaaaacgaccaatgaagcagtatctacattggggaatggagttcgagtgttggcaactgcagtgagagagctgaaagactttgtgagcaagaatttaactcgtgcaatcaacaaaaacaagtgcgacattgatgacctaaaaatggccgttagcttcagtcaattcaacagaaggtttctaaatgttgtgcggcaattttcagacaatgctggaataacaccagcaatatctttggacttaatgacagatgctgaactagccagggccgtttctaacatgccgacatctgcaggacaaataaaattgatgttggagaaccgcgcgatggtaagaaga

SEQ ID NO: 32 - DNA, 759 bp, human metapneumovirus; identical to SEQ ID NO: 31
  gene (1)...(759): human metapneumovirus, strain Quebec Isolate No C-74783

SEQ ID NO: 33 - DNA, 759 bp, human metapneumovirus
  gene (1)...(759): human metapneumovirus, strain Quebec Isolate No C-76837
atgtcttggaaagtggtgatcattttttcattgttaataacacctcaacacggtcttaaagagagctatttagaagagtcatgtagcactataactgaaggatatctcagtgttctgaggacaggttggtataccaacgtttttacactggaggtaggtgatgtagagaaccttacatgtgctgatggacctagcttaataaaaacagaattagatctgaccaaaagtgcactaagagagctcagaacagtttctgctgatcaactggcaagagaggagcaaattgagaatcccagacaatctagatttgttctaggagcaatagcactcggtgttgcaacagcagctgcagttacagcaggtgttgcaattgccaaaaccatccggcttgaaagtgaagtaacagcaattaagaatgccctcaaaaagaccaatgaagcagtatctacattggggaatggagttcgagtgttggcaactgcagtgagggaactgaaagattttgtgagcaagaatctaacacgtgcaatcaacaaaaacaagtgcgacattgctgacctgaaaatggccgttagcttcagtcaattcaacagaaggtttctaaatgttgtgcggcaattttcagacaatgctggaataacacccacaatatccttggacttaatgacagatgctgaactagccagagctgtttccaacatgccaacatctgcaggacaaataaaactgatgttggagaaccgtgcaatggtaagaaga

SEQ ID NO: 34 - DNA, 759 bp, human metapneumovirus
  gene (1)...(759): human metapneumovirus, strain Quebec Isolate No C-77748
atgtcttggaaagtggtgatcattttttcattgttaataacacttcaacacggtcttaaagagagctatttagaagagtcatgtagcactataactgaaggatatctcagtgttctgaggacaggttggtataccaacgtttttacactgaaggtaggtgatgtagagaaccttacatgtgctgatggacctagcttaataaaaacagaattagacctgaccaaaagtgcactaagagaactcagaacagtttctgctgatcaactggcaagagaggagcaaattgagaatcccagacaatctagatttgttctaggagcaatagcactcggtgttgcaacagcagctgcagttacagcaggtgttgcaattgccaaaaccatccggcttgaaagtgaagtaacagcaattaagaatgccctcaaaaagaccaatgaagcagtatctacattggggaatggagttcgagtgttggcaactgcagtgagggagctgaaagattttgtgagcaagaatctaacacgtgcaatcaacaaaaacaagtgcgacattgctgacctgaaaatggccgttagcttcagtcaattcaacagaaggtttctaaatgttgtgcggcaattttcagacaatgctggaataacaccagcaatatccttggacttaatgacagatgctgaactagccagagctgtttccaacatgccaacatctgcaggacaaataaaactgatgttggagaaccgtgcaatggtaagaaga

SEQ ID NO: 35 - DNA, 759 bp, human metapneumovirus
  gene (1)...(759): human metapneumovirus, strain Quebec Isolate No C-77807
atgtcttggaaagtggtgatcattttttcattgttaataacacttcaacacggtcttaaagagagctatttagaagagtcatgtagcactataactgaaggatatctcagtgttctgaggacaggttggtataccaacgtttttacactgaaggtaggtgatgtagagaaccttacatgtgctgatggacctagcttaataaaaacagaattagatctgaccaaaagtgcactaagagagctcagaccagtttctgctgatcaactggcaagagaggagcaaattgagaatcccagacaatctagatttgttctaggagcaatagcactcggtgttgcaacagcagctgcagttacagcaggtgttgcaattgccaaaaccatccggcttgaaagtgaagtaacagcaattaagaatgccctcaaaaagaccaatgaagcagtatctacattggggaatggagttcgagtgttggcaactgcagtgagggaactgaaagattttgtgagcaagaatctaacacgtgcaatcaacaaaaacaagtgcgacattgctgacctgaaaatggccgttagcttcagtcaattcaacagaaggtttctaaatgttgtgcggcaattttcagacaatgctggaataacaccagcaatatccttggacttaatgacagatgctgaactagccagagctgtttccaacatgccaacatctgcaggacaaataaaactgatgttggagaaccgtgcaatggtaagaaga

SEQ ID NO: 36 - DNA, 759 bp, human metapneumovirus
  gene (1)...(759): human metapneumovirus, strain Quebec Isolate No C-78132
atgtcttggaaagtggtgatcattttttcattgttaataacacctcaacacggtcttaaagagagctatttagaagagtcatgtagcactataactgaaggatatctcagtgttctgaggacaggttggtataccaacgtttttacactggaggtaggtgatgtagagaaccttacatgtgctgatggacctagcttaataaaaacagaattagatctgaccaaaagtgcactaagagagctcagaacagtttctgctgatcaactggcaagagaggagcaaattgagaatcccagacaatctagatttgttctaggagcaatagcactcggtgttgcaacagcagctgcagttacagcaggtgttgcaattgccaaaaccatccggcttgaaagtgaagtaacagcaattaagaatgccctcaaaaagaccaatgaagcagtatctacattggggaatggagttcgagtgttggcaactgcagtgagggaactgaaagattttgtgagcaagaatctaacacgtgcaatcaacaaaaacaagtgcgacattgctgacctgaaaatggccgttagcttcagtcaattcaacagaaggtttctaaatgttgtgcggcaattttcagacaatgctggaataacaccagcaatatccttggacttaatgacagatgctgaactagccagagctgtttccaacatgccaacatctgcaggacaaataaaactgatgttggagaaccgtgcaatggtaagaaga

SEQ ID NO: 37 - DNA, 759 bp, human metapneumovirus; identical to SEQ ID NO: 36
  gene (1)...(759): human metapneumovirus, strain Quebec Isolate No C-78163

SEQ ID NO: 38 - DNA, 759 bp, human metapneumovirus; identical to SEQ ID NO: 36
  gene (1)...(759): human metapneumovirus, strain Quebec Isolate No C-78406

SEQ ID NO: 39 - DNA, 759 bp, human metapneumovirus
  gene (1)...(759): human metapneumovirus, strain Quebec Isolate No hMPV-174
atgtcttggaaagtggtgatcattttttcattgttaataacacctcaacacggtcttaaagagagctatttagaagagtcatgtagcactataactgaaggatatctcagtgttctgaggacaggttggtataccaacgtttttacactggaggtaggtgatgtagagaaccttacatgtgctgatggacctagcttaataaaaacagaattagacctgaccaaaagtgcactaagagagctcagaacagtttctgctgatcaactggcaagagaggagcaaattgagaatcccagacaatctagatttgttctaggagcaatagcactcggtgttgcaacagcagctgcagttacagcaggtgttgcaattgccaaaaccatccggcttgaaagtgaagtaacagcaattaagaatgccctcaaaaagaccaatgaagcagtatctacattggggaatggagttcgagtgttggcaactgcagtgagagagctgaaagattttgtgagcaagaatctaacacgtgcaatcaacaaaaacaagtgcgacattgctgacctgaaaatggccgttagcttcagtcaattcaacagaaggtttctaaatgttgtgcggcaattttcagacaatgctggaataacaccagcaatatccttggacttaatgacagatgctgaactagccagagctgtttccaacatgccaacatctgcaggacaaataaaactgatgttggagaaccgtgcaatggtaagaaga

SEQ ID NO: 40 - DNA, 759 bp, human metapneumovirus; identical to SEQ ID NO: 39
  gene (1)...(759): human metapneumovirus, strain Quebec Isolate No hMPV-193

SEQ ID NO: 41 - DNA, 759 bp, human metapneumovirus
  gene (1)...(759): human metapneumovirus, strain Quebec Isolate No hMPV-194
atgtcttggaaagtggtgatcattttttcattgttaataacacctcaacacggttttaaagagagctatttagaagagtcatgtagcactataactgaaggatatctcagtgttctgaggacaggttggtataccaacgtttttacactggaggtaggtgatgtagagaaccttacatgtgctgatggacctagcttaataaaaacagaattagacctgaccaaaagtgcactaagagagctcagaacagtttctgctgatcaactggcaagagaggagcaaattgagaatcccagacaatctagatttgttctaggagcaatagcactcggtgttgcaacagcagctgcagttacagcaggtgttgcaattgccaaaaccatccggcttgaaagtgaagtaacagcaattaagaatgccctcaaaaagaccaatgaagcagtatctacattggggaatggagttcgagtgttggcaactgcagtgagagagctgaaagattttgtgagcaagaatctaacacgtgcaatcaacaaaaacaagtgcgacattgctgacctgaaaatggccgttagcttcagtcaattcaacagaaggtttctaaatgttgtgcggcaattttcagacaatgctggaataacaccagcaatatccttggacttaatgacagatgctgaactagccagagctgtttccaacatgccaacatctgcaggacaaataaaactgatgttggagaaccgtgcaatggtaagaaga

SEQ ID NO: 42 - DNA, 759 bp, human metapneumovirus; identical to SEQ ID NO: 39
  gene (1)...(759): human metapneumovirus, strain Quebec Isolate No hMPV-198

SEQ ID NO: 43 - DNA, 759 bp, human metapneumovirus; identical to SEQ ID NO: 39
  gene (1)...(759): human metapneumovirus, strain Quebec Isolate No hMPV-217

SEQ ID NO: 44 - DNA, 759 bp, human metapneumovirus
  gene (1)...(759): human metapneumovirus, strain Quebec Isolate No hMPV-208
atgtcttggaaagtggtgatcattttttcattgttaataacacctcaacacggtcttaaagagagctatttagaagagtcatgtagcactataactgaaggatatctcagtgttctgaggacaggttggtataccaacgtttttacactggaggtaggtgatgtagagaaccttacatgtgctgatggacctagcttaataaaaacagaattagacctgaccaaaagtgcactaagagagctcagaacagtttctgctgatcaactggcaagagaggagcaaattgagaatcccagacaatctagatttgttctaggagcaatagcactcggtgttgcaacagcagctgcagttacagcaggtgttgcaattgccaaaaccatccggcttgaaagtgaagtaacagcaattaagaatgccctcaaaaagaccaatgaagcagtatctacattggggaatggagttcgagtgttggcaactgcagtgagggagctgaaagattttgtgagcaagaatctaacacgtgcaatcaacaaaaacaagtgcgacattgctgacctgaaaatggccgttagcttcagtcaattcaacagaaggtttctaaatgttgtgcggcaattttcagacaatgctggaataacaccagcaatatccttggacttaatgacagatgctgaattagccagagctgtttccaacatgccaacatctgcaggacaaataaaactgatgttggagaaccgtgcaatggtaagaaga

SEQ ID NO: 45 - DNA, 759 bp, human metapneumovirus; identical to SEQ ID NO: 39
  gene (1)...(759): human metapneumovirus, strain Quebec Isolate No hMPV-223

SEQ ID NO: 46 - DNA, 759 bp, human metapneumovirus; identical to SEQ ID NO: 39
  gene (1)...(759): human metapneumovirus, strain Quebec Isolate No hMPV-228

SEQ ID NO: 47 - DNA, 759 bp, human metapneumovirus; identical to SEQ ID NO: 39
  gene (1)...(759): human metapneumovirus, strain Quebec Isolate No C-85885

SEQ ID NO: 48 - DNA, 759 bp, human metapneumovirus; identical to SEQ ID NO: 36
  gene (1)...(759): human metapneumovirus, strain Quebec Isolate No C-60298

SEQ ID NO: 49 - DNA, 759 bp, human metapneumovirus
  gene (1)...(759): human metapneumovirus, strain Quebec Isolate No C-63778
atgtcttggaaagtggtgattatcatttcgttactcataacacctcagcacggactaaaggaaagttatttagaagaatcatgtagtactataactgaaggatatctcagtgttttaagaacaggttggtacaccaatgtctttacattagaagttggtgatgttgaaaatcttacatgtactgatggacctagcttaatcaaaacagaacttgacctaaccaaaagtgctctaagagaactcaaaacagtttctgctgatcagttagcgagagaagaacaaattgaaaatcccagacaatcaaggtttgtcctaggtgcaatagctcttggtgttgccacagcagcagcagtcacagcaggcattgcgatagccaaaaccataaggcttgagagtgaagtgaatgcaatcaaaggtgctctcaaaacaaccaacgaggcagtatccacactaggaaatggagtgcgagtcctagccaccgcagtaagagagctgaaagaatttgtgagcaaaaacctgactagtgcaattaacaagaacaaatgtgacattgctgatctgaagatggctgtcagcttcagtcaattcaacagaagattcctaaatgttgtgcggcagttttcagacaatgcagggataacaccagcaatatcattggacctaatgactgatgctgagctggccagaactgtatcatacatgccaacatttgcaggacagataaaactaatgttagagaaccgtgcaatggtaagaaga

SEQ ID NO: 50 - DNA, 759 bp, human metapneumovirus
  gene (1)...(759): human metapneumovirus, strain Quebec Isolate No C-64199
atgtcttggaaagtggtgattatcatttcgttactcataacacttcagcacggactaaaagaaagttatttagaagaatcatgtagtactataactgaaggatatctcagtgttttaagaacaggttggtacaccaatgtctttacattagaagttggtgatgttgaaaatcttacatgtactgatggacctagcttaatcaaaacagaacttgacctaaccaaaagtgctctaagagaactcaaaacagtttctgctgatcagttagcgagagaagaacaaattgaaaatcccagacaatcaaggtttgtcctaggtgcaatagctcttggtgttgccacagcagcagcagtcacagcaggcattgcgatagccaaaaccataaggcttgagagtgaagtgaatgcaatcaaagatgctctcaaaacaaccaacgaggcagtatccacactaggaaatggagtgcgagtcctagccaccgcagtaagagagctgaaagaatttgtgagcaaaaacctgactagtgcaattaacaagaacaaatgtgacattgctgatctgaagatggctgtcagcttcagtcaattcaacagaagattcctaaatgttgtgcggcagttttcagacaatgcagggataacaccagcaatatcattggacctaatgactgatgctgagctggccagagctgtatcatacatgccaacatctgcaggacagataaaactaatgttagagaaccgtgcaatggtaagaaga

SEQ ID NO: 51 - DNA, 759 bp, human metapneumovirus
  gene (1)...(759): human metapneumovirus, strain Quebec Isolate No C-64200
atgtcttggaaagtggtgattatcatttcgttactcataacacctcagcacggactaaaagaaagttatttagaagaatcatgtagtactataactgaaggatatctcagtgttttaagaacaggttggtacaccaatgtctttacattagaagttggtgatgttgaaaatcttacatgtactgatggacctagcttaatcaaaacagaacttgacctaaccaaaagtgctctaagagaactcaaaacagtttctgctgatcagttagcgagagaagaacaaattgaaaatcccagacaatcaaggtttgtcctaggtgcaatagctcttggtgttgccacagcagcagcagtcacagcaggcattgcgatagccaaaaccataaggcttgagagtgaagtgaatgcaatcaaaggtgctctcaaaacaaccaacgaggcagtatccacactaggaaatggagtgcgagtcctagccaccgcagtaagagagctgaaagaatttgtgagcaaaaacctgactagtgcaattaacaagaacaaatgtgacattgctgatctgaagatggctgtcagcttcagtcaattcaacagaagattcctaaatgttgtgcggcagttttcagacaatgcagggataacaccagcaatatcattggacctaatgactgatgctgagctggccagagctgtatcatacatgccaacatctgcaggacagataaaactaatgttagagaaccgtgcaatggtaagaaga

SEQ ID NO: 52 - DNA, 759 bp, human metapneumovirus; identical to SEQ ID NO: 51
  gene (1)...(759): human metapneumovirus, strain Quebec Isolate No C-64536

SEQ ID NO: 53 - DNA, 759 bp, human metapneumovirus
  gene (1)...(759): human metapneumovirus, strain Quebec Isolate No C-73769
atgtcttggaaagtggtgattatcatttcgttactcataccacttcagcacggactaaaagaaagttatttagaagaatcatgtagtactataactgaaggatatctcagtgttttaagaccaggttggtacaccaatgtctttacattagaagttggtgatgttgaaaatcttacatgtactgatggacctagcttaatcaaaacagaacttgacctaaccaaaagtgctctaagagaactcaaaacagtttctgctgatcagttagcgagagaagaacaaattgaaaatcccagacaatcaaggtttgtcctaggtgcaatagctcttggtgttgccacagcagcagcagtcacagcaggcattgcgatagccaaaaccataaggcttgagagtgaagtgaatgcaatcaaaggtgctctcaaaacaaccaacgaggcagtatccacactaggaaatggagtgcgagtcctagccaccgcagtaagagagttgaaagaatttgtgagcaaaaacctgactagtgcaattaacaagaacaaatgtgacattgctgatctgaagatggctgtcagcttcagtcaattcaacagaagattcctaaatgttgtgcggcagttttcagacaatgcagggataacaccagcaatatcattagacctaatgactgatgctgagctggccagagctgtatcatacatgccaacatctgcaggacagataaaactaatgttagagaaccgtgcaatggtaagaaga

SEQ ID NO: 54 - DNA, 759 bp, human metapneumovirus
  gene (1)...(759): human metapneumovirus, strain Quebec Isolate No C-74501
atgtcttggaaagtggtgattatcatttcgttactcataacacttcagcacggactaaaagaaagttatttagaagaatcatgtagtactataactgaaggatatctcagtgttttaagaacaggttggtacaccaatgtctttacattagaagttggtgatgttgaaaatcttacatgtactgatggacctagcttaatcaaaacagaacttgacctaaccaaaagtgctctaagagaactcaaaacagtttctgctgatcagttagcgagagaagaacaaattgaaaatcccagacaatcaaggtttgtcctaggtgcaatagctcttggtgttgccacagcagcagcagtcacagcaggcattgcgatagccaaaaccataaggcttgagagtgaagtgaatgcaatcaaaggtgctctcaaaacaaccaacgaggcagtatccacactaggaaatggagtgcgagtcctagccactgcagtaagagagctgaaagaatttgtgagcaaaaacctgactagtgcaattaacaagaacaaatgtgacattgctgatctgaagatggctgtcagcttcagtcaattcaacagaagattcctaaatgttgtgcggcagttttcagacaatgcagggataacaccagcaatatcattagacctaatgactgatgctgagctggccagagctgtatcatacatgccaacatctgcaggacagataaaactaatgttagagaaccgtgcaatggtaagaaga

SEQ ID NO: 55 - DNA, 759 bp, human metapneumovirus
  gene (1)...(759): human metapneumovirus, strain Quebec Isolate No C-78429
atgtcttggaaagtggtgttcatcatttcgttactcataacaccccagcacgggctaaaggagagttatttggaagaatcatgcagtactataactgagggatacctcagtgttttaagaacaggctggtacattaatgtcttcacattagaagttggtgatgttgaaaatcttacatgtactgatggacctagcttaatcaaaacagaacttgacctaacaaaaagtgctttaagggaactcaaaacagtctctgctgatcagttagcgagagaggagcaaattgaaaatcccagacaatcaagatttgtcctaggtgcaatagctctcggagttgctacagcagcagcagtcacagcaggcattgcaatagccaaaaccataaggcttgagagtgaggtgaatgcaattaaaggtgctctcaaacaaactaatgaagcagtatccacattaggaaatggtgtgcgggtcctagccactgcagtgagagagctaaaagaatttgtgagcaaaaatctgactagtgcaatcaacaggaacaaatgtgacattgctgatctgaagatggctgtcagcttcagtcaattcaacagaagatttctcaatgttgtgcggcagttttcagacaatgcagggataacaccagcaatatcattggacctaatgactgatgctgaattggccagagctgtatcatacatgccaacatctgcaggacagataaaactgatgttggagaaccgtgcaatggtaagaaga

SEQ ID NO: 56 - DNA, 759 bp, human metapneumovirus
  gene (1)...(759): human metapneumovirus, strain Quebec Isolate No C-78652
atgtcttggaaagtggtgatcatcatttcgttactcataacaccccagcacgggctaaaggagagttatttggaagaatcatgcagtactataactgagggatacctcagtgttttaagaacgggctggtacactaatgtcttcacattagaagttggtgatgttgaaaatcttacatgtactgatggacctagcttaatcaaaacagaacttgacctaacaaaaagtgctttaagggaactcaaaacagtctctgctgatcagttaacgagagaggagcaaattgaaaatcccagacaatcaagatttgtcctaggtgcaatagctctcggagttgctacagcagcagcagtcacagcaggcattgcaatagccaaaaccataaggcttgagagtgaggtgaatgcaattaaaggtgctctcaaacaaactaatgaagcagtatccacattaggaaatggtgtgcgggtcctagccactgcagtgagagagctaaaagaatttgtgagcaaaaatctgactagtgcaatcaacaggaacaaatgtgacattgctgatctgaagatggctgtcagcttcagtcaattcaacagaagatttctaaatgttgtgcggcagttttcagacaatgcagggataacaccagcaatatcattggacctaatgactgatgctgaattggccagagctgtatcatacatgccaacatctgcaggacagataaaactgatgttggagaaccgtgcaatggtaagaaga

SEQ ID NO: 57 - DNA, 759 bp, human metapneumovirus
  gene (1)...(759): human metapneumovirus, strain Quebec Isolate No hMPV-118
atgtcttggaaaggggtgattatcatttcgttactcataacacctcagcacggactaaaagaaagttatttagaagaatcatgtagtactataactgaaggatatctcagtgttttaagaacaggttggtacaccaatgtctttacattagaagttggtgatgttgaaaatcttacatgtactgatggacctagcttaatcaaaacagaacttgacctaaccaaaagtgctctgagagaactcaaaacagtttctgctgatcagttaacgagagaagaacaaattgaaaatcccagacaatcaaggtttgtcctaggtgcaatagctcttggtgttgccacagcagcagcagtcacagcaggcattgcgatagccaaaaccataaggcttgagagtgaagtgaatgcaatcaaaggtgctctcaaaacaaccaacgaggcagtatccacactaggaaatggagtgcgagtcctagccaccgcagtaagagagctgaaagaatttgtgagcaaaaacctgaccagtgcaattaacaagaacaaatgtgacattgctgatctgaagatggctgtcagcttcagtcaattcaacagaagattcctaaatgttgtgcggcagttttcagacaatgcagggataacaccagcaatatcattagacctaatgactgatgctgagctggccagagctgtatcatacatgccaacatctgcagggcagataaaactaatgttagagaaccgtgcaatggtaagaaga

SEQ ID NO: 58 - DNA, 759 bp, human metapneumovirus
  gene (1)...(759): human metapneumovirus, strain Quebec Isolate No hMPV-200
atgtcttggaaagtggtgatcattttttcgttactcataacacctcagcacggactaaaagaaagttatttagaagaatcatgtagtactataactgaaggatatctcagtgttttaagaacaggttggtacaccaatgtctttacattagaagttggtgatgttgaaaatcttacatgtactgatggacctagcttaatcaaaacagaacttgacctaaccaaaagtgctctaagagaactcaaaacagtttctgctgatcagttaacgagagaagaacaaattgaaaatcccagacaatcaaggtttgtcctaggtgcaatagctcttggtgttgccacagcagcagcagtcacagcaggcattgcgatagccaaaaccataaggcttgagagtgaagtgaatgcaatcaaaggtgctctcaaaacaaccaacgaggcagtatccacactaggaaatggagtgcgagtcctagccaccgcagtaagagagctgaaagaatttgtgagcaaaaacctgaccagtgcaattaacaagaacaaatgtgacattgctgatctgaagatggctgtcagcttcagtcaattcaacagaagattcctaaatgttgtgcggcagttttcagacaatgcagggataacaccagcaatatcattagacctaatgactgatgctgagctggccagagctgtatcatacatgccaacatctgcagggcagataaaactaatgttagagaaccgtgcaatggtaagaaga

SEQ ID NO: 59 - DNA, 759 bp, human metapneumovirus
  gene (1)...(759): human metapneumovirus, strain Quebec Isolate No hMPV-176
atgtcttggaaagtggtgattatcatttcgttactcataacacctcagcacggactaaaagaaagttatttagaagaatcatgtagtactataactgaaggatatctcagtgttctaagaacaggttggtacaccaatgtctttacattagaagttggtgatgttgaaaatcttacatgtactgatggacctagcttaatcaaaacagaacttgacctaaccaaaagtgctttaagagaactcaaaacagtttctgctgatcagttagcgagagaagaacaaattgaaaatcccagacaatcaaggtttgtcctaggtgcaatagctctcggagttgccacagcagcagcagtcacagcaggcattgcaatagccaaaactataaggcttgagagtgaagtgaatgcaatcaaaggtgctctcaaaacaaccaatgaggcagtatcaacactaggaaatggagtgcgggtcctagccactgcagtaagagagctgaaagaatttgtgagcaaaaacctgactagtgcgatcaacaagaacaaatgtgacattgctgatttgaagatggctgtcagcttcagtcagttcaacagaagattcctaaatgttgtgcggcagttttcagacaatgcagggataacaccagcaatatcattggacctgatgactgatgctgagctggccagagctgtatcatacatgccaacatctgcaggacagataaaactaatgttagagaaccgtgcaatggtaagaaga

SEQ ID NO: 60 - DNA, 711 bp, human metapneumovirus
  gene (1)...(711): human metapneumovirus, strain Quebec Isolate No hMPV-193
atggaggtgaaagtggagaacattcgaacaatagatatgctcaaagcaagagtgaaaaatcgtgtggcacgcagcaaatgctttaaaaatgcctctttgatcctaataggaataactacattgagtatagccctcaatatctatctgatcataaactatacaatgcaagaaaacacatccgaatcagaacatcacaccagctcatcacccatggaatccagcagggaaactccaacggtccccatggactacccagacaccaatccaggctcacagtatccaactcaacagtccacagaaggctccacactctactttgcagcctcagcaagctcaccagagacagaaccaacatcaacaccaaacacaacaagccgcccgcccttcgtcgacacacacacaacaccaccaagtgcaagcagaacaaagacaagtccggcagtccacacaaaaaacaatccaaggataagcctcagaacacattccccaccatgggcaatgacaaggacggtccgcggaaccaccactctccgcacaagcagcacaagaaaaagaccgtctacagcatcagtccaacctgacagcagcgcaacaacccacaaacacgaagaagcaagcccagtgagcccgcaaacatctgcaagcacagcaagaccacaaaggaaggtcatggaggccagcacatcaacaacatacaatcaaactagttaa

SEQ ID NO: 61 - DNA, 711 bp, human metapneumovirus; identical to SEQ ID NO: 60
  gene (1)...(711): human metapneumovirus, strain Quebec Isolate No hMPV-228

SEQ ID NO: 62 - DNA, 711 bp, human metapneumovirus
  gene (1)...(711): human metapneumovirus, strain Quebec Isolate No C-77748
atggaggtgaaagtggagaacattcgaacaatagatatgctcaaagcaagagtgaaaaatcgtgtggcacgcagcaaatgctttaaaaatgcctctttgatcctaataggaataactacattgagtatagccctcaatatctatctaatcataaactatacaatgcaagaaaacacatccgaatcagaacatcacaccagctcatcacccatggaatccagcagggaaactccaacagtccctatggacaactcagacaccaatccaggctcacagtatccaactcaacagtccacagaaggctccacactctactttgcagcctcagcaagctcaccagagacagaaccaacatcaacaccagacacaacaagccgcccgcccttcgtcgacacacacacaacaccaccaagtgcaagcagaacaaggacaagtccggcagtccacacaaaaaacaatccaaggacaagccccagaacacattccccaccatgggcaatgacaaggacggtccgtggaaccaccactctccgcacaagcagcacaagaaaaagaccgtctacagcatcagtccaacctgacagcagcgcaacaacccacaaacacgaagaagcaagcccagtgagcccgcaaacatctgcgagcacagcaagaccacaaaggaagggcatggaggccagcacatcaacaacatacaaccaaactagttaa

SEQ ID NO: 63 - DNA, 711 bp, human metapneumovirus
  gene (1)...(711): human metapneumovirus, strain Quebec Isolate No C-78132
atggaggtgaaagtggagaacattcgaacaatagatatgctcaaagcaagagtgaaaaatcgtgtggcacgcagcaaatgctttaaaaatgcctctttgatcctaataggaataactacattgagtatagccctcaacatctatctgatcataaactatacaatgcaagaaaacacatccgaatcagaacatcacaccagctcatcacccatggaatccagcagggaaactccaacggtccctatagacaactcagacaccaatccaggctcacagtatccaactcaacagtccacagaaggctccacactccactttgcagcctcagcaagctcaccagagacagaaccaacatcaacaccagacacaacaagccgcccgcccttcgtcgacacacacacaacaccaccaagtgcaagcagaacaaggacaagtccggcagtccacacaaaaaacaatctaaagataagccccagaatacattccccaccatgggcaatgacaaggacggtccgtggaaccaccactctccgcacaagcagcacaagaaaaagaccgtctacagcatcagtccaacctgacagcagcacaacaactcacaaacacgaagaagcaagcccagtgagcccacaaacatctgcaagcacagcaagaccacaaaggaagggcatggaggccagcacatcaacaacatacaaccaaactagttaa

SEQ ID NO: 64 - DNA, 711 bp, human metapneumovirus
  gene (1)...(711): human metapneumovirus, strain Quebec Isolate No C-78406
atggaggtgaaagtggagaacattcgaacaatagatatgctcaaagcaagagtgaaaaatcgtgtggcacgcagcaaatgctttaaaaatgcctctttgatcctaataggaataactacattgagtatagccctcaacatctatctgatcataaactatacaatgcaagaaaacacatccgaatcagaacatcacaccagctcatcacccatggaatccagcagggaaactccaacggtccctatagacaactcagacaccaatccaggctcacagtatccaactcaacagtccacagaaggctccacactccactttgcagcctcagcaagctcaccagagacagaaccaacatcaacaccagacacaacaagccgcccgccctttgtcgacacacacacaacaccaccaagtgcaagcagaacaaggacaagtccggcagtccacacaaaaaacaatctaaagataagccccagaatacattccccaccatgggcaatgacaaggacggtccgtggaaccaccactctccgcacaagcagcacaagaaaaagaccgtctacagcatcagtccaacctgacagcagcacaacaactcacaaacacgaagaagcaagcccagtgagcccacaaacatctgcaagcacagcaagaccacaaaggaagggcatggaggccagcacatcaacaacatacaaccaaactagttaa

SEQ ID NO: 65 - DNA, 711 bp, human metapneumovirus
  gene (1)...(711): human metapneumovirus, strain Quebec Isolate No C-73750
atggaggtgaaagtggagaacattcgaacaatagatatgctcaaagcaagagtgaaaaatcgtgtggcacgcagcaaatgctttaaaaatgcctctttgatcctaataggaataactacattgagtatagccctcaacatctatctgatcataaactatacaatgcaagaaaacacatccgaatcagaacatcacaccagctcatcacccatggaatccagcagggaaactccaacggtccctatagacaactcagacaccaatccaggctcacagtatccaactcaacagtccacagaaggctccacactccactttgcagcctcagcaagctcaccagagacagaaccaacatcaacaccagacacaacaagccgcccgcccttcgtcgacacacacacaacaccaccaagtgcaagcagaacaaggacaagtccggcagtccacacaaaaaacaatctaaagataagccccagaacacattccccaccatgggcaatgacaaggacggtccgtggaaccaccactctccgcacaagcagcataagaaaaagaccgtccacagcatcagtccaacctgacagcagcacaacaacccacaaacacgaagaagcaagcccagtgagcccacaaacatctgcaagcacagcaagaccacaaaggaagggcatggaggccagcacatcaacaacacacaaccaaactagttaa

SEQ ID NO: 66 - DNA, 660 bp, human metapneumovirus
  gene (1)...(660): human metapneumovirus, strain Quebec Isolate No C-60298
atggaggtgaaagtggagaacattcgagcaatagacatgctcaaagcaagagtgaaaaatcgtgtggcacgtagcaaatgctttaaaaatgcttctttaatcctcataggaataactacactgagtatagctctcaatatctatctgatcataaactacacaatacaaaaaacctcatctgaatcagaacaccacaccagctcaccacccacagaatccaacaaggaagcttcaacaatctccacagacaacccagacatcaatccaaactcacagcatccaactcaacagtccacagaaaaccccacactcaaccccgcagcatcagtgagcccatcagaaacagaaccagcatcaacaccagacacaacaaaccgcctgtcctccgtagacaggtccacagcacaaccaagtgaaagcagaacaaagacaaaaccgacagtccacacaagaaacaacccaagcacagcttccagtacacaatccccaccacgggcaacaacgaaggcaatccgcagagccaccactttccgcatgagcagcacaggaaaaagaccaaccacaacatcagtccagtccgacagcagcaccacaacccaaaatcatgaagaaacaggttcagcgaacccacaggcatctgtaagcacaatgcaaaactag

SEQ ID NO: 67 - DNA, 654 bp, human metapneumovirus
  gene (1)...(654): human metapneumovirus, strain Quebec Isolate No C-74783
atggaggtgaaagtggagaacattcgagcaatagacatgctcaaagcaagagtgaaaaatcgtgtggcatgtagcaaatgctttaaaaatgcttctttaatcctcataggaataactacactgagtatagctctcaatatctatttgatcataaaccacacaatacaaaaaaccacatctgaatcagaacaccacaccagctcaccacccacagaatctaacaaggaagcttcaacaatctccacagacaacccagacatcaatccaaactcacagcatccaactcaacagtctacagaaaaccccacactcaaccccgcagtatcaacaagcccatcagaaacagaaccaacatcaacaccagacacaacaaaccgcctgtcctccgtagacaggtccacagcacaaccaagtgaaagcagaacaaagacaaaatcgacagtccacacaagaaacaacccaagcacagcttccagcacacaatccccaccacgggcaacaacgaaggcaatccgcagagccaccactttccgcatgagcagcacagaaaaaagaccaaccacaacactagtccagtacgacagcagcaccacaacccaaaatcatgaagaaacaggttcagcgaacccacaggcatctgcaagcacaatgtaa

SEQ ID NO: 68 - DNA, 660 bp, human metapneumovirus
  gene (1)...(660): human metapneumovirus, strain Quebec Isolate No C-74549
atggaggtgaaagtggagaacattcgagcaatagacatgctcaaagcaagagtgaaaaatcgtgtggcacgtagcaaatgcttcaaaaatgcttccccaatcctcacaggaataaccacactgagcatagctctcaatatccacctgatcataaactacacaatacaaaaaaccacatctgaatcagaacaccacaccagctcaccacccacagaatctaacaaggaagcttcaacaatctccacagacaacccagacatcaatccaaactcacagcatccaactcaacagtctacagaaaaccccacactcaaccccgcagtatcaacaagcccatcagaaacagaaccaacatcaacaccagacacaacaaaccgcctgtcctccgtagacaggtccacagcacaaccaagtgaaagcagaacaaagacaaaatcgacagtccacacaagaaacaacccaagcacagcttccagcacacaatccccaccacgggcaacaacgaaggcaatccgcagagccaccactttccgcatgagcagcacagaaaaaagaccaaccacaacattagtccagtacgacagcagcaccacaacccaaaatcatgaagaaacaggttcagcgaacccacaggcatctgtaagcacaatgtaaaactag

SEQ ID NO: 69 - DNA, 711 bp, human metapneumovirus
  gene (1)...(711): human metapneumovirus, strain Quebec Isolate No C-77807
atggaggtgaaagtggagaacattcgaacaatagatatgctcaaagcaagagtgaaaaatcgtgtggcacgcagcaaatgctttaaaaatgcctctttgatcctaataggaataacgacattgagtatagccctcaacatctatctgatcataaactatacaatgcaagaaaacacatccgaatcagaacatcacaccagctcatcacccatggaatccagcagggaaactccaacggtccctatagacaactcagacaccaatccaggctcacagtatccaactcaacagtccacagaaggctccacactccactttgcagcctcagcaagctcaccagagacagaaccaacatcaacaccagacacaacaagccgcccgcccttcgtcgacacacacacaacaccaccaagtgcaagcagaacaaggacaagtccggcagtccacacaaaaaacaatctaaagataagccccagaatacattccccaccatgggcaatgacaaggacggtccgtggaaccaccactctccgcacaagcagcacaagaaaaagaccgtctacagcatcagtccaacctgacagcagcacaacaactcacaaacacgaagaagcaagcccagtgagcccacaaacatctgcaagcacagcaagaccacaaaggaagggcatggaggccagcacatcaacaacatacaaccaaactagttaa

SEQ ID NO: 70 - DNA, 711 bp, human metapneumovirus; identical to SEQ ID NO: 63
  gene (1)...(711): human metapneumovirus, strain Quebec Isolate No C-78163

SEQ ID NO: 71 - DNA, 711 bp, human metapneumovirus
  gene (1)...(711): human metapneumovirus, strain Quebec Isolate No C-68661
atggaggtgaaagtggagaacattcgaacaatagatacgctcaaagcaagagtgaaaaatcgtgtggcacgcagcaaatgctttaaaaatgcctctttgatcctaataggaataactacattgagtatagccctcaatatctatctgatcataaactatacaatgcaagaaaacacatccaaatcagaacatcacaccagctcatcacccatggaatccagcagggaaactccaacggtccctatagacaactcagacaccaatccaggctcacagtatccaactcaacagtccacagaaggctccacactccactttgcagcctcagcaagctcaccagagacagaaccaacatcaacaccagacacaacaagccgcccgcccttcgtcgacacacacacaacaccaccaagtgcaagcagaacaaggacaagtccggcagtccacacaaaaaacaatccaaggataagccccagaacacattccccaccatgggcaatgacaaggacggtccgtggaaccaccactctccgcacaagcagcacaagaaaaagaccgtccacagcatcagtccaacccgacagcagcgcaacaacccacaaacacgaagaagcaagcccagtgagcccacaaacatctgcaagcacagcaagaccacaaaggaagggcatggaggccagcacatcaacaacatacaaccaaactagttaa

SEQ ID NO: 72 - DNA, 711 bp, human metapneumovirus
  gene (1)...(711): human metapneumovirus, strain Quebec Isolate No C-88448
atggaggtgaaagtggagaacattcgaacaatagatatgctcaaagcaagagtgaaaaatcgtgtggcacgcagcaaatgctttaaaaatgcctctttgatcctaataggaataactacattgagtatagccctcaatatctatctaatcataaactatacaatgcaagaaaacacatccgaatcagaacatcacaccagctcatcacccatggaatccagcagggaaactccaacagtccctatggacaactcagacaccaatccaggctcacagtatctaactcaacagtccacagaaggctccacactctactttgcagcctcagcaagctcaccagagacagaaccaacatcaacaccagacacaacaagccgcccgcccttcgtcgacacacacacaacaccaccaagtgcaagcagaacaaggacaagtccggcagtccacacaaaaaacaatccaaggacaagccccagaacacattccccaccatgggcaatgacaaggacggtccgtggaaccaccactctccgcacaagcagcacaagaaaaagaccgtccacagcatcagtccaacctgacagcagcgcaacaacccacaaacacgaagaagcaagcccagtgagcccgcaaacatctgcgagcacagcaagaccacaaaggaagggcatggaggccagcacatcaacaacatacaaccaaactagttaa

SEQ ID NO: 73 - DNA, 711 bp, human metapneumovirus
  gene (1)...(711): human metapneumovirus, strain Quebec Isolate No C-88470
atggaggtgaaagtggagaacattcgaacaatagatatgctcaaagcaagagtgaaaaatcgtgtggcacgcagcaaatgctttaaaaatgcctctttgatcctaataggaataactacattgagtatagccctcaatatctatctgatcataaactatacaatgcaagaaaacacatccgaatcagaacatcacaccagctcatcacccatggaatccagcagggaaactccaacggtccccatggactacccagacaccaatccaggctcacagcatccaactcaacagtccacagaaggctccacactctactttgcagcctcagcaagctcaccagagacagaaccaacatcaacaccaaacacaacaagccgcccgcccttcgtcgacacacacacaacaccaccaagtgcaagcagaacaaagacaagtccggcagtccacacaaaaaacaatccaaggataagcctcagaacacattccccaccatgggcaatgacaaggacggtccgcggaaccaccactctccgcacaagcagcacaagaaaaagaccgtctacagcatcagtccaacctgacagcagcgcaacaacccacaaacacgaagaagcaagcccagtgagcccgcaaacatctgcaagcacagcaagaccacaaaggaaggtcatggaggccagcacatcaacaacatacaaccaaactagttaa

SEQ ID NO: 74 - DNA, 711 bp, human metapneumovirus
  gene (1)...(711): human metapneumovirus, strain Quebec Isolate No C-86316
atggaggtgaaagtggagaacattcgaacaatagatatgctcaaagcaagagtgaaaaatcgtgtggcacgcagcaaatgctttaaaaatgcctctttgatcctaataggaataactacattgagtatagccctcaatatctatctaatcataaactatacaatgcaagaaaacacatccgaatcagaacatcacaccagctcatcacccatggaatccagcaaggaaactccaacagtccctatggacaactcagacaccaatccaggctcacagtatccaactcaacagtccacagaaggctccacactctactttgcagcctcagcaagctcaccagagacagaaccaacatcaacaccagacacaacaagccgcccgcccttcgtcgacacacacacaacaccaccaagtgcaagcagaacaaggacaagtccggcagtccacacaaaaaacaatccaaggacaagcctcagaacacattccccaccatgggcaatgacaaggacggtccgtggaaccaccactctccgcacaagcagcacaagaaaaagaccgtccacagcatcagtccaacctgacagcagcgcaacaacccacaaacacgaagaagcaagcccagtgagcccgcaaacatctgcgagcacagcaagaccacaaaggaagggcatggaggccagcacatcaacaacatacaaccaaactagttaa

SEQ ID NO: 75 - DNA, 6017 bp, human metapneumovirus
  gene (1)...(6017): human metapneumovirus, strain 00-1 accession number af371337
atggatcctctcaatgaatccactgttaatgtctatcttcctgactcatatcttaaaggagtgatttcctttagtgagactaatgcaattggttcatgtctcttaaaaagaccttacctaaaaaatgacaacactgcaaaagttgccatagagaatcctgttatcgagcatgttagactcaaaaatgcagtcaattctaagatgaaaatatcagattacaagatagtagagccagtaaacatgcaacatgaaattatgaagaatgtacacagttgtgagctcacattattaaaacagtttttaacaaggagtaaaaatattagcactctcaaattaaatatgatatgtgattggctgcagttaaagtctacatcagatgatacctcaatcctaagttttatagatgtagaatttatacctagctgggtaagcaattggtttagtaattggtacaatctcaacaagttgattctggaattcaggaaagaagaagtaataagaactggttcaatcttgtgtaggtcattgggtaaattagtttttgttgtatcatcatatggatgtatagtcaagagcaacaaaagcaaaagagtgagcttcttcacatacaatcaactgttaacatggaaagatgtgatgttaagtagattcaatgcaaatttttgtatatgggtaagcaacagtctgaatgaaaatcaagaagggctagggttgagaagtaatctgcaaggcatattaactaataagctatatgaaactgtagattatatgcttagtttatgttgcaatgaaggtttctcacttgtgaaagagttcgaaggctttattatgagtgaaattcttaggattactgaacatgctcaattcagtactagatttagaaatactttattaaatggattaactgatcaattaacaaaattaaaaaataaaaacagactcagagttcatggtaccgtgttagaaaataatgattatccaatgtacgaagttgtacttaagttattaggagatactttgagatgtattaaattattaatcaataaaaacttagagaatgctgctgaattatactatatatttagaatattcggtcacccaatggtagatgaaagagatgcaatggatgctgtcaaattaaacaatgaaatcacaaaaatccttaggtgggagagcttgacagaactaagaggggcattcatattaaggattatcaaaggatttgtagacaacaacaaaagatggcccaaaattaaaaacttaaaagtgcttagtaagagatggactatgtacttcaaagcaaaaagttaccccagtcaacttgaattaagcgaacaagattttttagagcttgctgcaatacagtttgaacaagagttttctgtccctgaaaaaaccaaccttgagatggtattaaatgataaagctatatcacctcctaaaagattaatatggtctgtgtatccaaaaaattacttacctgagaaaataaaaaatcgatatctagaagagactttcaatgcaagtgatagtctcaaaacaagaagagtactagagtactatttgaaagataataaattcgaccaaaaagaacttaaaagttatgttgttaaacaagaatatttaaatgataaggatcatattgtctcgctaactggaaaagaaagagaattaagtgtaggtagaatgtttgctatgcaaccaggaaaacagcgacaaatacaaatattggctgaaaaattgttagctgataatattgtaccttttttcccagaaaccttaacaaagtatggtgatctagatcttcagagaataatggaaatcaaatcggaactttcttctattaaaactagaagaaatgatagttataataattacattgcaagagcatccatagtaacagatttaagtaagttcaaccaagcctttaggtatgaaactacagcgatctgtgcggatgtagcagatgaactacatggaacacaaagcctattctgttggttacatcttatcgtccctatgacaacaatgatatgtgcctatagacatgcaccaccagaaacaaaaggtgaatatgatatagataagatagaagagcaaagtggtttatatagatatcatatgggtggtattgaaggatggtgtcaaaaactctggacaatggaagctatatctctattagatgttgtatctgtaaaaacacgatgtcaaatgacatctttattaaacggtgacaaccaatcaatagatgtaagtaaaccagttaagttatctgagggtttagatgaagtgaaagcagattatagcttggctgtaaaaatgttaaaagaaataagagatgcatacagaaatataggccataaacttaaagaaggggaaacatatatatcaagagatcttcagtttataagtaaggtgattcaatctgaaggagtaatgcatcctacccctataaaaaagatcttaagagtgggaccatggataaacacaatattagatgacattaaaaccagtgcagagtcaatagggagtctatgtcaggaattagaatttaggggggaaagcataatagttagtctgatattaaggaatttttggctgtataatttatacatgcatgaatcaaagcaacaccccctagcagggaagcagttattcaaacaactaaataaaacattaacatcagtgcagagattttttgaaataaaaaaggaaaatgaagtagtagatctatggatgaacataccaatgcagtttggaggaggagatccagtagtcttctatagatctttctatagaaggacccctgattttttaactgaagcaatcagtcatgtggatattctgttaagaatatcagccaacataagaaatgaagcgaaaataagtttcttcaaagccttactgtcaatagaaaaaaatgaacgtgctacactgacaacactaatgagagatcctcaagctgttggctcagagcgacaagcaaaagtaacaagtgatatcaatagaacagcagttaccagcatcttaagtctttctccaaatcaacttttcagcgatagtgctatacactacagtagaaatgaagaagaggtcggaatcattgctgacaacataacacctgtttatcctcatggactgagagttttgtatgaatcattaccttttcataaagctgaaaaagttgtgaatatgatatcaggaacgaaatccataaccaacttattacagagaacatctgctattaatggtgaagatattgacagagctgtatccatgatgctggagaacctaggattattatctagaatattgtcagtagttgttgatagtatagaaattccaaccaaatctaatggtaggctgatatgttgtcagatatctagaaccctaagggagacatcatggaataatatggaaatagttggagtaacatcccctagcatcactacatgcatggatgtcatatatgcaactagctctcatttgaaagggataatcattgaaaagttcagcactgacagaactacaagaggtcaaagaggtccaaagagcccttgggtagggtcgagcactcaagagaaaaaattagttcctgtttataacagacaaattctttcaaaacaacaaagagaacagctagaagcaattggaaaaatgagatgggtatataaagggacaccaggtttaagacgattactcaataagatttgtcttggaagtttaggcattagttacaaatgtgtaaaacctttattacctaggtttatgagtgtaaatttcctacacaggttatctgtcagtagtagacctatggaattcccagcatcagttccagcttatagaacaacaaattaccattttgacactagtcctattaatcaagcactaagtgagagatttgggaatgaagatattaatttggtcttccaaaatgcaatcagctgtggaattagcataatgagtgtagtagaacaattaactggtaggagtccaaaacagttagttttaatacctcaattagaagaaatagacattatgccaccaccagtgtttcaagggaaattcaattataagctagtagataagataacttctgatcaacatatcttcagtccagacaaaatagatatgttaacactggggaaaatgctcatgcccactataaaaggtcagaaaacagatcagttcctgaacaagagagagaattatttccatgggaataatcttattgagtctttgtcagcagcgttagcatgtcattggtgtgggatattaacagagcaatgtatagaaaataatattttcaagaaagactggggtgacgggttcatatcggatcatgcttttatggacttcaaaatattcctatgtgtctttaaaactaaacttttatgtagttgggggtcccaagggaaaaacattaaagatgaagatatagtagatgaatcaatagataaactgttaaggattgataatactttttggagaatgttcagcaaggttatgtttgaatcaaaggttaagaaaaggataatgttatatgatgtaaaatttctatcattagtaggttatatagggtttaagaattggtttatagaacagttggatcagctgagttgcatgaggtaccttggattgtcaatgccgaaggtgatctggttgagatcaagtcaattaaaatctatttgcaactgatagagcaaagtttatttttaagaataactgttttgaactatacagatatggcacatgctctcacaagattaatcagaaagaagttgatgtgtgataatgcactattaactccgattccatccccaatggttaatttaactcaagttattgatcctacagaacaattagcttatttccctaagataacatttgaaaggctaaaaaattatgacactagttcaaattatgctaaaggaaagctaacaaggaattacatgatactgttgccatggcaacatgttaatagatataactttgtctttagttctactggatgtaaagttagtctaaaaacatgcattggaaaacttatgaaagatctaaaccctaaagttctgtactttattggagaaggggcaggaaattggatggccagaacagcatgtgaatatcctgacatcaaatttgtatacagaagtttaaaagatgaccttgatcatcattatcctttggaataccagagagttataggagaattaagcaggataatagatagcggtgaagggctttcaatggaaacaacagatgcaactcaaaaaactcattgggatttgatacacagagtaagcaaagatgctttattaataactttatgtgatgcagaatttaaggacagagatgatttttttaagatggtaattctatggaggaaacatgtattatcatgcagaatttgcactacttatgggacagacctctatttattcgcaaagtatcatgctaaagactgcaatgtaaaattacctttttttgtgagatcagtagccacctttattatgcaaggtagtaaactgtcaggctcagaatgctacatactcttaacactaggccaccacaacaatttaccctgccatggagaaatacaaaattctaagatgaaaatagcagtgtgtaatgatttttatgctgcaaaaaaacttgacaataaatctattgaagccaactgtaaatcacttttatcagggctaagaataccgataaataagaaagaattaaatagacagagaaggttattaacactacaaagcaaccattcttctgtagcaacagttggaggtagcaaggtcatagagtctaaatggttaacaaacaaggcaaacacaataattgattggttagaacatattttaaattctccaaaaggtgaattaaattatgatttttttgaagcattagaaaatacttaccctaatatgattaaactaatagataatctagggaatgcagagataaaaaaactgatcaaagtaactggatatatgcttgtaagtaaaaaatga

SEQ ID NO: 76 - DNA, 450 bp, human metapneumovirus
  gene (1)...(450): human metapneumovirus, strain Quebec Isolate No hMPV-118
taaaatgaaaatatcagattacaaggtagtagaaccagtaaacatgcaacatgaaataatgaagaatgtacacagctgtgagcacacactattgaaacagtttttaacaaggagtaaaaacattagcactctcaaattaaatatgatatgtgattggctgcaattaaaatctacatcagatggtacctcaatcctaagttttatagatgtagaatttatacctagttgggtaagcaactggtttagtaattggtacaatctcaataagttaattttagaattcaggagagaagaagtaataagaaccggttcaatcttgtgcaggtcattaggtaaattagtttttattgtatcatcatatggatgtatcgtcaagagcaacaaaagcaaaagagtgagcttctttacatacaatcaactgttaacatggaaagatgtgatgttaagtcg

SEQ ID NO: 77 - DNA, 450 bp, human metapneumovirus
  gene (1)...(450): human metapneumovirus, strain Quebec Isolate No hMPV-163
ttgaaatatcagattacaggatagtagagccagtaaacatgcaacatgaaattatgaagaatgtacacagttgtgagctcacattattaaaacagtttttaacaaggagtaaaaatattagcactctcaaattaaatatgatatgtgattggctgcagttaaaatctacatcagatgatacctcaatcttaagttttatagatgtagaatttatacctagctgggtaagcaattggtttagtaattggtacaatctcaacaagttgattctggaattcaggaaagaagaagtaataagaactggttcaatcttgtgtaggtcattaggtaaattagtttttgttgtatcatcatatggatgtatagtcaagagcaacaaaagcaaaagagtgagcttcttcacatacaatcaactgttaacatggaaagatgtgatgttaagtagattca

SEQ ID NO: 78 - DNA, 500 bp, human metapneumovirus
  gene (1)...(500): human metapneumovirus, strain Quebec Isolate No hMPV-174
gagaatcctgttatcgagcatgttagactcaaaaatgcaatcaatctaaagatgaaaatatcagattacaggatagtagagccagtaaacatgcaacatgaaattatgaagaatgtacacagttgtgagctcacattattaaaacagtttttaacaaggagtaaaaatattagcactctcaaattaaatatgatatgtgattggctgcagttaaagtctacatcagatgatacctcaatcttaagttttatagatgtagaatttatacctagctgggtaagcaattggtttagtaattggtacaatctcaacaagttgattctggaattcaggaaagaagaagtaataagaactggttcaatcttgtgtaggtcattgggtaaattagtttttgttgtatcatcatatggatgtatagtcaagagcaacaaaagcaaaagagtgagcttcttcacatacaatcaactgttaacatggaaagatgtgatgttaagtagatt

SEQ ID NO: 79 - DNA, 450 bp, human metapneumovirus
  gene (1)...(450): human metapneumovirus, strain Quebec Isolate No hMPV-184
attgaaatatcagattacaggatagtagagccagtaaacatgcaacatgaaattatgaagaatgtacacagttgtgagctcacattattaaaacagtttttaacaaggagtaaaaatattagcactctcaaattaaatatgatatgtgattggctgcagttaaagtctacatcagatgatacctcaatcttaagttttatagatgtagaatttatacctagctgggtaagcaattggtttagtaattggtacaatctcaacaagttgattctggaattcaggaaagaagaagtaataagaactggttcaatcttgtgtaggtcattaggtaaattagtttttgttgtatcatcatatggatgtatagtcaagagcaacaaaagcaaaagagtgagcttcttcacatacaatcaactgttaacatggaaagatgtgatgttaagtagattc

SEQ ID NO: 80 - DNA, 500 bp, human metapneumovirus
  gene (1)...(500): human metapneumovirus, strain Quebec Isolate No hMPV-196
ttgcccatagagaatcctgttattgagcatgtgagactcaaaaatgcagtcaattctaaaatgaaaatatcagattacaaggtagtagagccagtaaacatgcaacatgaaataatgaagaatgtacacagttgtgagctcacactattgaaacagtttttaacaaggagtaaaaacattagcactctcaaattaaatatgatatgtgattggctgcaattaaagtctacatcagatgatacctcaatcctaagtttcatagatgtagaatttatacctagttgggtaagcaactggtttagtaattggtacaatctcaataagttaattttggaattcagaagagaagaagtaataagaaccggttcaatcttatgcaggtcattgggtaaattagtttttattgtatcatcatatggatgtatcgtcaagagcaacaaaagcaaaagagtgagcttcttcacatacaatcaactgttaacatggaaagatgtgatgtt

SEQ ID NO: 81 - DNA, 450 bp, human metapneumovirus
  gene (1)...(450): human metapneumovirus, strain Quebec Isolate No C-76837
agatgaaaatatcagattacaggatagtagagccagtaaacatgcaacatgaaattatgaagaatgtacacagttgtgagctcacattattaaaacagtttttaacaaggagtaaaaatattagcactctcaaattaaatatgatatgtgattggctgcagttaaagtctacatcagatgatacctcaatcttaagttttatagatgtagaatttatacctagctgggtaagcaattggtttagtaattggtacaatctcaacaagttgattctggaattcaggaaagaagaagtaataagaactggttcaatcttgtgcaggtctttgggtaaattagtttttgttgtatcatcatatggatgtatagtcaagagcaacaaaagcaaaagagtgagcttcttcacatacaatcaactgttaacatggaaagatgtgatgttaagtagat

SEQ ID NO: 82 - DNA, 450 bp, human metapneumovirus
  gene (1)...(450): human metapneumovirus, strain Quebec Isolate No C-77807
taagatgaaaatatcagattacaggatagtagagccagtaaacatgcaacatgaaattatgaagaatgtacacagttgtgagctcacattattaaaacagtttttaacaaggagtaaaaatattagcactctcaaattaaatatgatatgtgattggctgcagttaaagtctacatcagatgatacctcaatcttaagttttatagatgtagaatttatacctagctgggtaagcaattggtttagtaattggtacaatctcaacaagttgattctggaattcaggaaagaagaagtaataagaactggttcaatcttgtgtaggtcattgggtaaattagtttttgttgtatcatcatacggatgtatagtcaagagcaacaaaagcaaaagagtgagcttcttcacatacaatcaactgttaacatggaaagatgtgatgttaagtag

SEQ ID NO: 83 - DNA, 450 bp, human metapneumovirus
  gene (1)...(450): human metapneumovirus, strain Quebec Isolate No hMPV-185
taaaattgaaatatcagattacaaggtagtagagccagtaaacatgcaacatgaaataatgaagaatgtacacagttgtgagctcacactattgaaacagtttttaacaaggagtaaaaacattagcactctcaaattaaatatgatatgtgattggctgcaattaaagtctacatcagatgatacctcaatcctaagtttcatagatgtagaatttatacctagttgggtaagcaactggtttagtaattggtacaatctcaataagttaattttggaattcagaagagaagaagtaataagaaccggttcaatcttatgcaggtcattgggtaaattagtttttattgtatcatcatatggatgtatcgtcaagagcaacaaaagcaaaagagtgagcttcttcacatacaatcaactgttaacatggaaagatgtgatgttaagtag

SEQ ID NO: 84 - DNA, 500 bp, human metapneumovirus
  gene (1)...(500): human metapneumovirus, strain Quebec Isolate No C-78123
atcctgttatcgagcatgttagactcaaaaatgcaatcaattctaagatgaaaatatcagattacaggatagtagagccagtaaacatgcaacatgaaattatgaagaatgtacacagttgtgagctcacattattaaaacagtttttaacaaggagtaaaaatattagcactctcaaattaaatatgatatgtgattggctacagttaaagtctacatcagatgatacctcaatcttaagttttatagatgtagaatttatacctagctgggtaagcaattggtttagtaattggtacaatctcaacaagttgattctggaattcaggaaagaagaagtaataagaactggttcaatcttgtgcaggtctttgggtaaattagtttttgttgtatcatcatatggatgtatagtcaagagcaacaaaagcaaaagagtgagcttcttcacatacaatcaactgttaacatggaaagatgtgatgttaagtagattcaat

SEQ ID NO: 85 - DNA, 450 bp, human metapneumovirus
  gene (1)...(450): human metapneumovirus, strain Quebec Isolate No C-91205
caattctaaaatgaaaatatcagattacaaggtagtagagccagtaaacatgcaacatgaaataatgaagaatgtacacagttgtgagctcacactattgaaacagtttttaacaaggagtaaaaacattagcactctcaaattaaatatgatatgtgattggctgcaattaaagtctacatcagatgatacctcaatcctaagtttcatagatgtagaatttatacctagttgggtaagcaactggtttagtaattggtacaatctcaataagttaattttggaattcagaagagaagaagtaataagaaccggttcaatcttatgcaggtcattgggtaaattagtttttattgtatcatcatatggatgtattgtcaagagcaacaaaagcaaaagagtgagcttcttcacatacaatcaactgttaacatggaaagatgtgatgtt

SEQ ID NO: 86 - DNA, 400 bp, human metapneumovirus
  gene (1)...(400): human metapneumovirus, strain Quebec Isolate No C-91364
ttgcatagagaatcctgttattgagcatgtcagactcaaaaatggcagtaattctaaaatgaaaatatcagattacaaggtagtagaaccagtaaacatgcaacatgaaataatgaagaatgtacacagctgtgagcacacactattgaaacagtttttaacaaggagtaaaaacattagcactctcaaattaaatatgatatgtgattggctgcaattaaaatctacatcagatggtacctcaatcctaagttttatagatgtagaatttatacctagttgggtaagcaactggtttagtaattggcacaatctcaataagttaattttagaattcaggagagaagaagtaataagaaccggttcaatcttgtgcaggtcattaggtaaattagttttt

SEQ ID NO: 87 - DNA, 500 bp, human metapneumovirus
  CDS (1)...(500): human metapneumovirus, strain Quebec Isolate No C-91749
atagagaatcctgttattgagcatgtgagactcaaaaatgcagtcaattctaaaatgaaaatatcagattacaaggtagtagagccagtaaacatgcaacatgaaataatgaagaatgtacacagttgtgagctcacactattgaaacagtttttaactaggagtaaaaacattagcactctcaaattaaatatgatatgtgattggctgcaattaaagtctacatcagatgatacctcaatcctaagtttcatagatgtagaatttatacctagttgggtaagcaactggtttagtaattggtacaatctcaataagttaattttggaattcagaagagaagaagtaataagaaccggttcaatcttttgcaggtcattgggtaaattagtttttattgtatcatcatatggatgtatcgtcaagagcaacaaaagcaaaagagtgagcttcttcacatacaatcaactgttaacatggaaagatgtgatgttaagtng

SEQ ID NO: 88 - DNA, 450 bp, human metapneumovirus
  gene (1)...(450): human metapneumovirus, strain Quebec Isolate No hMPV-Q86
aattctaaaatggaaatatcagattacaaggtagtagaaccaataaacatgcaacatgaaataatgaagaatgtacacagttgtgagctcacactattgaaacagtttttaacaaggagtaaaaacattagcactctcaaattaaatatgatatgtgattggctgcaattaaaatctacatcagatgatacctcaatcctaagttttatagatgtagaatttatacctagttgggtaagcaactggtttagtaattggtacaatctcaataagttaattttagaattcaggagagaggaagtaataagaaccggttcaatcttgtgcaggtcattaggtaaattagtttttattgtatcatcatatggatgtatcgtcaagagcaacaaaagcaaaagagtgagcttcttcacatacaatcaactgttaacatggaaagatgtgatgtta

SEQ ID NO: 89 - DNA, 450 bp, human metapneumovirus
  gene (1)...(450): human metapneumovirus, strain Quebec Isolate No hMPV-Q97
aaaatgaaaatatcagattacaaggtagtagagccagtaaacatgcaacatgaaataatgaagaatgtacacagttgtgagctcacactattgaaacagtttttaactaggagtaaaaacattagcactctcaaattaaatatgatatgtgattggctgcaattaaagtctacatcagatgatacctcaatcctaagtttcatagatgtagaatttatacctagttgggtaagcaactggtttagtaattggtacaatctcaataagttaattttggaattcagaagagaagaagtaataagaaccggttcaatcttatgcaggtcattgggtaaattagtttttattgtatcatcatatggatgtatcgtcaagagcaacaaaagcaaaagagtgagcttcttcacatacaatcaactgttaacatggaaagatgtgatgttaagtaga

SEQ ID NO: 90 - DNA, 537 bp, human metapneumovirus
  gene (1)...(537): human metapneumovirus, strain Can 97-83
agaatcctgttattgagcatgtgagactcaaaaatggcggtcattctaaaatgaaaatatcagattacaaggtagtagagccagtaaacatgcaacatgaaataatgaagaatgtacacagttgtgagctcacactattgaaacagtttttaacaaggagtaaaaacattagcactctcaaattgaatatgatatgtgattggctgcaattaaagtctacatcagatgatacctcaatcctaagtttcatagatgtagaatttatacctagttgggtaagcaactggtttagtaattggtacaatctcaataagttaattttggaattcagaagagaggaagtaataagaaccggttcaatcttatgcaggtcattgggtaaattagtttttattgtatcatcatatggatgtatcgtcaagagcaacaaaagcaaaagagtgagcttcttcacatacaatcaactgttaacatggaaagatgtgatgttaagtagatttaatgcgaatttttgtatatgggtaagcaacagttctg

SEQ ID NO: 91 - DNA, 20 bp, human metapneumovirus
  primer no 55
atgggacaagtgaaaatgtc

SEQ ID NO: 92 - DNA, 19 bp, human metapneumovirus
  primer no 56
gagtctcagtacacaataa

SEQ ID NO: 93 - DNA, 18 bp, human metapneumovirus
  primer no 57
gtcactgcagttgttgta

SEQ ID NO: 94 - DNA, 19 bp, human metapneumovirus
  primer no 58
gcatttccgagaacaacac

SEQ ID NO: 95 - DNA, 19 bp, human metapneumovirus
  primer no 18 multiplex assay
gagtctcagtacacaataa

SEQ ID NO: 96 - DNA, 19 bp, human metapneumovirus
  primer no 19 multiplex assay
gcatttccgagaacaacac

SEQ ID NO: 97 - DNA, 18 bp, human metapneumovirus
  primer no 59
atgtcttggaaagtggtg

SEQ ID NO: 98 - DNA, 17 bp, human metapneumovirus
  primer no 60
tcttcttaccattgcac

SEQ ID NO: 99 - DNA, 20 bp, human metapneumovirus
  primer no 61
ctaattatgtggtatgaagc

SEQ ID NO: 100 - DNA, 18 bp, human metapneumovirus
  primer no 62
atggagtcctacctagta

SEQ ID NO: 101 - DNA, 19 bp, human metapneumovirus
  primer no 63
aaggtgcttggttgttatc

SEQ ID NO: 102 - DNA, 19 bp, human metapneumovirus
  primer no 64
atgtcattccctgaaggaa

SEQ ID NO: 103 - DNA, 19 bp, human metapneumovirus
  primer no 65
ttaactggtaaatctcatc

SEQ ID NO: 104 - DNA, 19 bp, human metapneumovirus
  primer no 66
catgggacaagtagttatg

SEQ ID NO: 105 - DNA, 19 bp, human metapneumovirus
  primer no 67
atggaggtgaaagtggaga

SEQ ID NO: 106 - DNA, 19 bp, human metapneumovirus
  primer no 68
ttaactagtttggttgtat

SEQ ID NO: 107 - DNA, 19 bp, human metapneumovirus
  primer no 69
cttagagttattttgtatt

SEQ ID NO: 108 - DNA, 24 bp, human metapneumovirus
  primer no 70
ggacaagtagttatggaggtgaaa

SEQ ID NO: 109 - DNA, 28 bp, human metapneumovirus
  primer no 71
ctcctttaagatatgagtcaggmagrta

SEQ ID NO: 110 - DNA, 22 bp, human metapneumovirus
  primer no 20 multiplex assay
gttgccatagagaatcctgtta

SEQ ID NO: 111 - DNA, 22 bp, human metapneumovirus
  primer no 21 multiplex assay
cattcagactgttgcttaccca

SEQ ID NO: 112 - DNA, 22 bp, human metapneumovirus
  primer no 72
gttgccatagagaatcctgtta

SEQ ID NO: 113 - DNA, 22 bp, human metapneumovirus
  primer no 73
cattcagactgttgcttaccca

SEQ ID NO: 114 - DNA, 27 bp, human Metapneumovirus
  human Metapneumovirus primer A1
ccaattactaaaccaattgcttaccca

SEQ ID NO: 115 - DNA, 21 bp, human metapneumovirus
  probe no 74
gcaagcttatggggccggtca

SEQ ID NO: 116 - DNA, 25 bp, human metapneumovirus
  probe no 75
acaatgctaaggtggggggtcattg

SEQ ID NO: 117 - DNA, 23 bp, human metapneumovirus
  probe no 76
gttctgaggacaggttggtacac

SEQ ID NO: 118 - DNA, 23 bp, human metapneumovirus
  probe no 77
aatgtttttacactggaggtagg

SEQ ID NO: 119 - DNA, 22 bp, human metapneumovirus
  probe no 78
gttacaataccagcattcatca

SEQ ID NO: 120 - DNA, 25 bp, human metapneumovirus
  probe no 79
atcagtttcaatcaaagagagtgag

SEQ ID NO: 121 - DNA, 25 bp, human metapneumovirus
  probe no 80
taaagcaagcaatcaaagtcatgga

SEQ ID NO: 122 - DNA, 25 bp, human metapneumovirus
  probe no 81
cccattgaagaagaagagtctaccg

SEQ ID NO: 123 - DNA, 20 bp, human metapneumovirus
  probe no 82
actcaagcccacagcatcca

SEQ ID NO: 124 - DNA, 23 bp, human metapneumovirus
  probe no 83
ctcaacagtccacagaaggctcc

SEQ ID NO: 125 - DNA, 26 bp, human metapneumovirus
  probe no 84
attggtttagtaattggtacaatctc

SEQ ID NO: 126 - DNA, 26 bp, human metapneumovirus
  probe no 85
acaagttgattctggaattcaggaaa

SEQ ID NO: 127 - DNA, 25 bp, human Metapneumovirus
  human Metapneumovirus probe A2
cgagcatgttagactcaaaaatgca

SEQ ID NO: 128 - DNA, 23 bp, human metapneumovirus
  probe no 33 multiplex assay
atgtatccgtccaagctgagtta

SEQ ID NO: 129 - DNA, 26 bp, human metapneumovirus
  probe no 34 multiplex assay
attggtttagtaattggtacaatctc

SEQ ID NO: 130 - DNA, 1027 bp, human Influenza A virus
  gene (1)...(1027): human Influenza A virus strain A/Hong Kong/1180/99(H3N2) accession number af386771
agcaaaagcaggtagatattgaaagatgagccttctacccgaggtcgaaacgtatgttctctctatcgttccatcaggccccctcaaagccgaaatcgcgcagagacttgaagatgtctttgctgggaaaaacacagatcttgaggctctcatggaatggctaaagacaagaccaatcctgtcacctctgactaaggggattttagggtttgtgttcacgctcaccgtgcccagtgagcgaggactgcagcgtagacgctttgtccaaaatgccctcaatgggaatggggatccaaataacatggacaaagcagttaaactgtatagaaaacttaagagggagataacattccatggggccaaagaaatagcactcagttattctgctggtgcacttgccagttgcatgggcctcatatacaataggatgggggctgtaaccactgaagtggcatttggcctggtatgtgcaacatgtgaacagattgctgactcccagcacaggtctcataggcaaatggtggcaacaaccaatccattaataagacatgagaacagaatggttttggccagcactacagctaaggctatggagcaaatggctggatcaagtgagcaggcagcggaggccatggagattgctagtcaggccaggcaaatggtgcaggcaatgagagccgttgggactcatcctagctccagtactggtctaagagatgatcttcttgaaaatttgcagacctatcagaaacggatgggggtgcagatgcaacgattcaagtgacccgcttgttgttgccgcgagtatcattgggatcttgcacttgatattgtggattcttgatcgtctttttttcaaatgcatctatcgactcttcaaacacggccttaaaagagggccttctacggaaggagtacctgagtctatgagggaagaatatcgaaaggaacagcagaatgctttggatgctgaccacagtcattttgtcagcataaagttggagtaaaaaactaccttgtttctact

SEQ ID NO: 131 - DNA, 987 bp, human Influenza A virus
  gene (1)...(987): human Influenza A virus strain A/Charlottesville/31/95 (H1N1) accession number af398876
atgagtcttctaaccgaggtcgaaacgtacgttctctctatcgtcccgtcaggccccctcaaagccgagatcgcacagagacttgaagatgtctttgctggaaagaacaccgatcttgaggctctcatggagtggctaaagacaagaccgatcctgtcacctctgactaaggggattttaggatttgtgttcacgctcaccgtgcccagtgagcgaggactgcagcgtagacgctttgtccaaaatgcccttaatgggaatggggatccaaataacatggacagagcagttaaactgtatagaaagcttaagagggagataacattccatggggccaaagaaatagcactcagttattctgctggtgcacttgccagttgtatgggcctcatatacaacaggatgggggctgtgaccaccgaatcagcatttggcctgatatgcgcaacctgtgaacagattgctgactcccagcataagtctcataggcaaatggtaacaacaaccaatccattaataagacatgagaacagaatggttctggccagcactacagctaaagctatggagcaaatggctggatcgagtgaacaagcagctgaggccatggaggttgctagtcaggccaggcaaatggtgcaggcaatgagagccattgggactcatcctagctctagcactggtctgaaaaatgatcttcttgaaaatttgcaggcctatcagaaacgaatgggggtgcagatgcaacgattcaagtgatcctcttgttgttgccgcaagtatcattgggattgtgcacctgatattgtggattattgatcgcctttttttcaaatgcatttatcgtatctttaaacacggtctgaaaagagggccttctacggaaggagtaccagagtctatgagggaagaatatcgagaggaacagcagaatgctgtggatgctgacgaaggtcattttgtcagcatagagctggagtaaaaaac

SEQ ID NO: 132 - DNA, 1076 bp, human Influenza B virus
  gene (1)...(1076): human Influenza B virus strain B/Nagoya/20/99
atgtcgctgtttggagacacaattgcctacctgctttcattgacggaagatggagaaggcaaagcagaactagcagaaaaattacactgttggttcggtgggaaagaatttgacctagactctgccttggaatggataaaaaacaaaagatgcttaactgatatacaaaaagcactaattggtgcctctatctgctttttaaaacccaaagaccaagaaagaaaaagaagattcatcacagagcccctatcaggaatgggaacaacagcaacaaaaaagaaaggcctgattctagctgagagaaaaatgagaagatgtgtgagctttcatgaagcatttgaaatagcagaaggccatgaaagctcagcgctactatattgtctcatggtcatgtacctgaaccctggaaattattcaatgcaagtaaaactaggaacgctctgtgctttgtgcgagaaacaagcatcacattcacacagggctcatagcagagcagcgagatcttcagtgcctggagtgaggcgagaaatgcaaatggtctcagctatgaacacagcaaaaacaatgaatggaatgggaaagggagaagacgtccaaaaactggcagaagaactgcaaagcaacattggagtgttgagatctcttggagcaagtcaaaagaatggggaaggaattgcaaaggatgtaatggaagtgctaaagcagagctctatgggaaattcagctcttgtgaagaaatacctataatgcttgaaccatttcagattctttcaatttgttcttttatcttatcagctctccatttcatggcttggacaatagggcatttgagtcaaataaaaagaggagtaaacatgaaaatacgaataaaaggtccaaataaagagacaataaacagagaagtatcaattttgagacacaattaccaaaaagaaatccaggccaaagaaacaatgaaggaagtgctctctgacaacatagaagtattgagtgaccacatagtgattgaggggctttctgctgaagagataataaaaatgggcaaaacagttttggaggtagaaaaattgcattaa

SEQ ID NO: 133 - DNA, 1725 bp, human Respiratory Syncitial Virus
  gene (1)...(1725): human Respiratory Syncitial Virus strain B1 accession number af013254
atggagctgctgatccacaggttaagtgcaatcttcctaactcttgctattaatgcattgtacctcacctcaagtcagaacataactgaggagttttaccaatcgacatgtagtgcagttagcagaggttattttagtgctttaagaacaggttggtataccagtgtcataacaatagaattaagtaatataaaagaaaccaaatgcaatggaactgacactaaagtaaaacttataaaacaagaattagataagtataagaatgcagtgacagaattacagctacttatgcaaaacacaccagctgccaacaaccgggccagaagagaagcaccacagtatatgaactatacaatcaataccactaaaaacctaaatgtatcaataagcaagaagaggaaacgaagatttctgggcttcttgttaggtgtaggatctgcaatagcaagtggtatagctgtatccaaagttctacaccttgaaggagaagtgaacaagatcaaaaatgctttgttatctacaaacaaagctgtagtcagtctatcaaatggggtcagtgttttaaccagcaaagtgttagatctcaagaattacataaataaccaattattacccatagtaaatcaacagagctgtcgcatctccaacattgaaacagttatagaattccagcagaagaacagcagattgttggaaatcaacagagaattcagtgtcaatgcaggtgtaacaacacctttaagcacttacatgttaacaaacagtgagttactatcattgatcaatgatatgcctataacaaatgatcagaaaaaattaatgtcaagcaatgttcagatagtaaggcaacaaagttattctatcatgtctataataaaggaagaagtccttgcatatgttgtacagctacctatctatggtgtaatagatacaccttgctggaaattacacacatcacctctatgcaccaccaacatcaaagaaggatcaaatatttgtttaacaaggactgatagaggatggtattgtgataatgcaggatcagtatccttctttccacaggctgacacttgtaaagtacagtccaatcgagtattttgtgacactatgaacagtttgacattaccaagtgaagtcagcctttgtaacactgacatattcaattccaagtatgactgcaaaattatgacatcaaaaacagacataagcagctcagtaattacttctcttggagctatagtgtcatgctatggtaaaactaaatgcactgcatccaacaaaaatcgtgggattataaagacattttctaatggttgtgactatgtgtcaaacaaaggagtagatactgtgtcagtgggcaacactttatactatgtaaacaagctggaaggcaagaacctttatgtaaaaggggaacctataataaattactatgaccctctagtgtttccttctgatgagtttgatgcatcaatatctcaagtcaatgaaaaaatcaatcaaagtttagcttttattcgtagatctgatgaattactacataatgtaaatactggcaaatctactacaaatattatgataactacaattattatagtaatcattgtagtattgttatcattaatagctattggtttgctgttgtattgcaaagccaaaaacacaccagttacactaagcaaagaccaactaagtggaatcaataatattgcattcagcaaatag

SEQ ID NO: 134 - DNA, 1575 bp, human Parainfluenza 1 virus
  gene (1)...(1575): human Parainfluenza 1 virus    strain Washington/1964 accession number nc003461
atggccgggctactaagtacttttgacacatttagttccaggagaagtgagagcatcaataagtctggcggaggagcaattatacctggtcaaagaagtaccgtttctgtcttcacattaggcccgagtgtgacagatgatgcagataaattattaatagcaaccactttcttagcccactcattggacacagataaacaacactctcaaagaggaggatttttagtatcactccttgcaatggcctacagtagtccggagttatatctcactacaaacggtgtcaatgctgatgtcaagtatgtgatatacaatatagagagagatcctaaaagaacaaaaacagatgggttcattgtcaaaacaagagacatggagtatgaaagaacaacagagtggttgtttggacctatgatcaacaagaacccattgttccaagggcaaagagagaatgcggatctagaggcattgcttcagacatatggatatcctgcatgtctcggagctataatagttcaagtttggatagtgttggttaaagccataacaagtagtgctggtctaagaaaaggattcttcaatagattagaagcattcagacaggatggaaccgttaaaagtgccctggtcttcacaggagacacagttgaaggtattggtgcagtgatgaggtcacaacaaagcttagtatctcttatggtagaaactctggtgactatgaacacatccaggtcagatttaactacattagagaagaacattcagattgtaggaaattacataagagatgcaggattagcatctttcatgaacaccatcaagtatggtgtagaaacgaagatggccgccctgacactatcaaatctgagaccagatataaacaaattgagaagccttgttgatatctatctatcaaagggagcccgagccccttttatatgtatactcagagacccagttcatggagactttgcccctggaaactatccagcactgtggagctacgcaatgggcgttgctgtggtacaaaacaaagctatgcaacagtatgtaactggaagaacatatttggacatggaaatgttcctacttggacaagctgtagctaaagatgctgattccaaaatcagcagtgctctggaggaagaactaggtgtgacagatacagcaaaagagagactaagacaccatctgacaaacctttcaggaggggatggtgcgtaccacaagcctacaggtggtggagctatagaagtggcaattgatcatacagacataacatttggagtcgaggacactgctgatcgggacaacaagaactggacaaatgacagcaatgaaagatggatgaatcactcgatcagcaaccacacaatcacgattcgtggtgcagaagaacttgaagaagagacaaatgatgaagacatcactgatatagaaaacaagattgcacgaaggctggccgacagaaaacagagactaagccaggcaaacaataaacgagacaccagcagtgatgctgactatgagaatgatgatgatgctacagcggctgcagggataggaggaatttaa

SEQ ID NO: 135 - DNA, 1548 bp, human Parainfluenza 3 virus
  gene (1)...(1548): human Parainfluenza 3 virus strain GP accession number ab012132
atgttgagcctatttgatacatttaatgcacgtaggcaagaaaacataacaaaatcagccggtggagctatcattcctggacagaaaaatactgtctctatatttgctcttggaccgacaataactgatgataatgagaaaatgacattagctcttctttttttatctcattcactggataatgagaaacaacatgcacaaagggcagggttcctggtgtccttattatcaatggcttatgccaatccagagctctacctaacaacaaatggaagtaatgcagatgtcaagtatgtcatatacatgatcgagaaagatctaaaacggcaaaagtatggaggatttgtggttaagacgagagagatggtatatgacaagacaactgattggatatttggaagtgacctggattgtgatcaggaaactatgttgcagaacggaagaaacaattcaacaattgaagaccttgtccacacatttgggtatccatcatgtttaggagctcttataatacagatctggatagttctggtcaaagctatcactagtatctcagggttaagaaaaggctttttcacccgattggaagctttcagacaagatggaacagtgcaggcagggctggtattgagcggtgacacagtggatcagattgggtcaattatgcggtctcaacagagcttggtaactcttatggttgaaacattaataacaatgaatactagcagaaatgacctcacaaccatagaaaagaatatacaaattgttggtaactatataagagatgcaggtctcgcttcattcttcaatacaatcagatatggaattgagactagaatggcagctttgactctatccactctcagaccagacatcaatagattaaaagctttgatggaactgtatttatcaaagggaccacgcgctcctttcatctgtatcctcagagatcccatacatggtgagttcgcaccaggcaactatcctgccatatggagctatgcaatgggagtggcagttgtacaaaatagagccatgcaacagtatgtgacgggaagatcatatctggatattgatatgttccagctaggacaagcagtagcacgtgatgctgaagctcagatgagctcaacactagaagatgaacttggagtgacacacgaagctaaagaaagcttgaagagacatataaggaacataaatagttcagagacatctttccacaaaccaacaggtggatcagccatagagatggcaatagatgaagagccagaacaattcgaacatagatcagatcaagaacgagatggagaacctcaatcatctataattcaatatgcttgggcagaaggaaacagaagcgatgatcggactgagcaagatacagaatctgacaatatcaagactgaacaacaaaacatcagagacagactaaacaagagactcaacgaaaagaagaaacaaggcagtcaaccacctaccaatcccacaaacagaacgaatcaggacgaaatagatgatctgtttaatgcatttggaagcaactaa

SEQ ID NO: 136 - DNA, 2910 bp, human Adenovirus
  gene (1)...(2910): human Adenovirus strain 105 accession number aj293903
atggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggatcgcgaggataccgcgtactcgtacaaggcgcggttcaccctggctgtgggtgacaaccgcgtgcttgatatggcttccacgtactttgacatccgcggcgtgctggacaggggccctactttcaagccctactctggcactgcctacaacgctctagctcccaagggcgctcccaactcctgtgagtgggaacaaaccgaagatagcggccgggcggttgccgaggatgaagaagaagaggaagatgaagatgaagaagaggaagaggaagagcaaaacgctcgagatcaggctactaagaaaacacatgtttatgcccaggctcctttgtctggagaaacaattacaaaaagcgggctacaaataggatcagacaatgcagaaacacaagctaaacctgtatatgcagatccttcctatcaaccagaacctcaaattggcgaatctcagtggaacgaagctgatgctaatgcagcaggagggagagtgcttaaaaaaacaactcccatgaaaccatgctatggatcttatgccaggcctacaaatccttttggtggccaatccgtcctggttccggatgaaaaaggggtgcctcttccaaaggttgacttgcaattcttctcaaatactacctctttgaacgaccggcaaggcaatgccactaaaccaaaagtggtgttgtacagtgaagatgtaaatttggaaaccccagacacacatctgtcttacaaacctggaaaaggtgatgaaaattctaaagctatgttgggtcaacaatctatgccaaacagacccaattacatcgctttcagggacaattttattggcctaatgtattataacagcactggcaacatgggtgttcttgctggtcaggcatcgcagctaaatgccgtggtagatttgcaagacagaaacacagagctgtcctatcaacttttgcttgattccataggtgatagaaccagatatttttctatgtggaatcaggctgtagacagctatgacccagatgttagaatcattgaaaaccatggaactgaggatgaattgccaaattattgtttccctcttgggggtattggggtaactgacacctatcaagctattaaggctaatggcaatggcgcaggtgataatggaaatactacatggacaaaagatgaaacttttgcaacacgtaatgaaataggagtgggtaacaactttgccatggaaattaacctaaatgccaacctatggagaaatttcctttactccaatattgcactatacctgccagacaagctaaaatacaaccccaccaacgtggaaatatctgacaaccccaacacctacgactacatgaacaagcgagtggtggctcctgggcttgtagactgctacattaaccttggggcgcgctggtctctggactacatggacaacgttaatccctttaaccaccaccgcaatgcgggcctgcgttaccgctccatgttgttgggaaacggacgctacgtgccttttcacattcaggtgccccaaaagttttttgccattaaaaacctcctcctcctgccaggctcatacacatatgaatggaacttcaggaaggatgttaacatggttctgcagagctctctgggaaacgaccttagagttgacggggctagcattaagtttgacagcatttgtctttacgccaccttcttccccatggcccacaacacggcctccacgctggaagccatgctcagaaatgacaccaacgaccagtcctttaatgactacctttccgccgccaacatgctatatcccatacccgccaacgccaccaacgtgcccatctccatcccatcgcgcaactgggcagcatttcgcggttgggccttcacacgcttgaagacaaaggaaaccccttccctgggatcaggctacgacccttactacacctactctggctccataccataccttgacggaaccttctatcttaatcacacctttaagaaggtggccattacttttgactcttctgttagctggccgggcaacgaccgcctgcttactcccaatgagtttgagattaagcgctcagttgacggggagggctataacgtagctcagtgcaacatgacaaaggactggttcctagtgcagatgttggccaactacaatattggctaccagggcttctacattccagaaagctacaaagaccgcatgtactcgttcttcagaaacttccagcccatgagccggcaagtggtggacgatactaaatacaaagattatcagcaggttggaattatccaccagcataacaactcaggcttcgtaggctacctcgctcccaccatgcgcgagggacaagcttaccccgctaatgttccctaccccctaataggcaaaaccgcggttgatagtattacccagaaaaagtttctttgcgaccgcaccctgtggcgcatcccattctccagtaactttatgtccatgggtgcgctcacagacctgggccaaaaccttctctacgcaaactccgcccacgcgctagacatgacctttgaggtggatcccatggacgagcccacccttctttatgttttgtttgaagtctttgacgtggtccgtgtgcaccagccgcaccgcggcgtcatcgagaccgtgtacctgcgcacgcccttctcggccggcaacgccacaacataa

SEQ ID NO: 137 - DNA, 810 bp, human Enterovirus
  gene (1)...(810): human Enterovirus strain Coxakievirus B1 accession number m16560
cggcccctgaatgcggctaatcctaactgcggagcacataccctcaaaccagggggcagtgtgtcgtaacgggcaactctgcagcggaaccgactactttgggtgtccgtgtttcattttattcctatactggctgcttatggtgacaattgacaggttgttaccatatagttattggattggccatccggtgactaacagagcaattatatatctctttgttgggtttataccacttagcttgaaagaggttaaaacactacatctcatcattaaactaaatacaacaaaatgggggctcaagtatcaacacaaaagactggagcacatgagaccgggctgaatgcaagcgggaattccatcatccattacacaaacataaactactataaagatgctgcatccaactcagccaacagacaggatttcactcaagacccaggtaaatttactgagccagtgaaggacattatgatcaaatcgatgccagcattgaactccccctctgccgaagagtgtggttatagtgacagggttcgatcaataaccctcgggaactccactatcacaacacaggagtgtgctaatgttgtggttgggtatggtgtgtggccagaatatttgaaggacaatgaagccacaggcgaggaccaaccaacacaacccgacgtggccacatgtcggttctacactttagagtccgtgcagtggatgaagaattcagcaggttggtggtggaagttaccagatgcactttcacaaatggggttgtttggacaaaatatgcagtaccactacttagggagaaca

SEQ ID NO: 138 - DNA, 681 bp, human Rhinovirus
  gene (1)...(681): human Rhinovirus strain 14 accession number k02121
taaccctagagccttatgccacgatccagtggttgtaaggtcgtaatgagcaattccgggacgggaccgactactttgggtgtccgtgtttctcatttttcttcatattgtcttatggtcacagcatatatatacatatactgtgatcatgggcgctcaggtttctacacagaaaagtggatctcacgaaaatcaaaacattttgaccaatggatcaaatcagactttcacagttataaattactataaggatgcagcaagtacatcatcagctggtcaatcactgtcaatggacccatctaagtttacagaaccagttaaagatctcatgcttaagggtgcaccagcattgaattcacccaatgttgaggcctgtggttatagtgatagagtacaacaaatcacactcgggaattcaacaataacaacacaagaagcagccaacgctgttgtgtgttatgctgaatggccagagtaccttccagatgtggacgctagtgatgtcaataaaacttcaaaaccagacacttctgtctgtaggttttacacattggatagtaagacatggacaacaggttctaaaggctggtgctggaaattaccagatgcactcaaggatatgggtgtgttcgggcaaaacatgtttttccactcactaggaagatcaggttacacagtaca

SEQ ID NO: 139 - DNA, 21 bp, human Influenza A virus
  primer no 12 multiplex assay
cttctaaccgaggtcgaaacg

SEQ ID NO: 140 - DNA, 24 bp, human Influenza A virus
  primer no 13 multiplex assay
agggcattttggacaaagcgtcta

SEQ ID NO: 141 - DNA, 20 bp, human Influenza B virus
  primer no 14 multiplex assay
ttacactgttggttcggtgg

SEQ ID NO: 142 - DNA, 20 bp, human Influenza B virus
  primer no 15 multiplex assay
gccagtttttggacgtcttc

SEQ ID NO: 143 - DNA, 23 bp, human Respiratory Syncytial Virus
  primer no 16 multiplex assay
aacagtttaacattaccaagtga

SEQ ID NO: 144 - DNA, 23 bp, human Respiratory Syncytial virus
  primer no 17 multiplex assay
tcattgacttgagatattgatgc

SEQ ID NO: 145 - DNA, 26 bp, human Parainfluenza 1 virus
  primer no 22 multiplex assay
tctggcggaggagcaattatacctgg

SEQ ID NO: 146 - DNA, 26 bp, human Parainfluenza 1 virus
  primer no 23 multiplex assay
atctgcatcatctgtcacactcgggc

SEQ ID NO: 147 - DNA, 22 bp, human Parainfluenza 1 virus
  human Parainfluenza 1 virus primer P1
gtatggtgtagaaacgaagatg

SEQ ID NO: 148 - DNA, 22 bp, human Parainfluenza 1 virus
  human Parainfluenza 1 virus primer P2
ttgtaccacagcaacacccatt

SEQ ID NO: 149 - DNA, 26 bp, human Parainfluenza 3 virus
  primer no 24 multiplex assay
aacaggaaactatgctgcagaacggc

SEQ ID NO: 150 - DNA, 26 bp, human Parainfluenza 3 virus
  primer no 25 multiplex assay
gatccactgtgtcaccgctcaatacc

SEQ ID NO: 151 - DNA, 25 bp, human Parainfluenza virus
  humna Parainfluenza virus primer P3
tcttgcttcattcttcaatacaatc

SEQ ID NO: 152 - DNA, 23 bp, human Parainfluenza 3 virus
  human Parainfluenza 3 virus primer P4
aattccatcagagcttttaatct

SEQ ID NO: 153 - DNA, 18 bp, human Adenovirus
  primer human Adenovirus P5
actccctcgatgatgccg

SEQ ID NO: 154 - DNA, 26 bp, human Adenovirus
  primer no 26 multiplex assay
cctacgcacgatgtgaccacagaccg

SEQ ID NO: 155 - DNA, 26 bp, human Adenovirus
  primer no 27 multiplex assay
gtgttgtaggcagtgccggagtaggg

SEQ ID NO: 156 - DNA, 21 bp, human Sars Coronavirus
  primer SARS no P5
ctaacatgcttaggataatgg

SEQ ID NO: 157 - DNA, 21 bp, human Sars Coronavirus
  human Sars Coronavirus primer P6
caggtaagcgtaaaactcatc

SEQ ID NO: 158 - DNA, 24 bp, human Sars Coronavirus
  human Sars Coronavirus primer P7
atgaattaccaagtcaatggttac

SEQ ID NO: 159 - DNA, 22 bp, human Sars Coronavirus
  human Sars Coronavirus primer P8
cataaccagtcggtacagctac

SEQ ID NO: 160 - DNA, 16 bp, human Enterovirus/human Rhinovirus
  primer no 28 multiplex assay
ctactttgggtgtccg

SEQ ID NO: 161 - DNA, 19 bp, Artificial Sequence
  primer no 29 multiplex assay
ggtaattccaccaccancc

SEQ ID NO: 162 - DNA, 23 bp, human Influenza A virus
  probe no 30 multiplex assay
tcttcaagtctctgtgcgatctc

SEQ ID NO: 163 - DNA, 23 bp, human Influenza B virus
  probe no 31 multiplex assay
agctcagcgctactatattgtct

SEQ ID NO: 164 - DNA, 23 bp, human Respiratory Syncytial virus
  probe no 32 multiplex assay
ccttccagcttgtttacatagta

SEQ ID NO: 165 - DNA, 23 bp, human Parainfluenza 1 virus
  probe no 35 multiplex assay
aagtaccgtttctgtcttcacat

SEQ ID NO: 166 - DNA, 23 bp, human Parainfluenza 3 virus
  probe no 36 multiplex assay
cctgcactgttccatcttgtctg

SEQ ID NO: 167 - DNA, 18 bp, human Adenovirus
  probe no 37 multiplex assay
cgcgtactcgtacaaggc

SEQ ID NO: 168 - DNA, 23 bp, human Rhinovirus
  probe no 38 multiplex assay
attcagcataacacacaacagcg

SEQ ID NO: 169 - DNA, 23 bp, human Enterovirus
  probe no 39 multiplex assay
cctgtggcttcattgtccttcaa

SEQ ID NO: 170 - DNA, 558 bp, human Herpesvirus 2
  modified human Herpesvirus 2 internal control multiplex no 1
ttacactgttggttcggtggcgactttgccagcctgtaccccagcatcatccaggcccacaacctgtgcttcagtacgctctccctgcggcccgaggccgtcgcgcacctggaggcggaccgggactacctggagatcgaggtggggggccgacggctgttcttcgtgaaggcccacgtacgcgagagcctgctgagcatcctgctgcgcgactggctggccatgcgaaagcagatccgctcgcggatcccccagagcacccccgaggaggccgtcctcctcgacaagcaacaggccgccatcaaggtggtgtgcaactcggtgtacgggttcaccggggtgcagcacggtcttctgccctgcctgcacgtggccgccaccgtgacgaccatcggccgcgagatgctcctcgcgacgcgcgcgtacgtgcacgcgcgctgggcggagttcgatcagctgctggccgactttccggaggcggccggcatgcgcgcccccggtccgtactccatgcgcatcatctacggggacacggactgaagacgtccaaaaactggc

SEQ ID NO: 171 - DNA, 554 bp, human Herpesvirus 2
  modified human Herpesvirus 2 internal control multiplex no 2
ctactttgggtgtccgcgactttgccagcctgtaccccagcatcatccaggcccacaacctgtgcttcagtacgctctccctgcggcccgaggccgtcgcgcacctggaggcggaccgggactacctggagatcgaggtggggggccgacggctgttcttcgtgaaggcccacgtacgcgagagcctgctgagcatcctgctgcgcgactggctggccatgcgaaagcagatccgctcgcggatcccccagagcacccccgaggaggccgtcctcctcgacaagcaacaggccgccatcaaggtggtgtgcaactcggtgtacgggttcaccggggtgcagcacggtcttctgccctgcctgcacgtggccgccaccgtgacgaccatcggccgcgagatgctcctcgcgacgcgcgcgtacgtgcacgcgcgctgggcggagttcgatcagctgctggccgactttccggaggcggccggcatgcgcgcccccggtccgtactccatgcgcatcatctacggggacacggactggctggtggtggaaattacc

SEQ ID NO: 172 - DNA, 23 bp, human Herpesvirus
  Internal Control probe no 42 multiplex assay
acaccgagttgcacaccaccttg

SEQ ID NO: 173 - DNA, 13335 bp, Viruses
  CAN9783 genomic DNA
acgcgaaaaaaacgcgtataaattaagttacaaaaaaacatgggacaagtgaaaatgtctcttcaagggattcacctgagtgatctatcatacaagcatgctatattaaaagagtctcagtatacaataaagagagatgtaggcacaacaacagcagtgacaccctcatcattgcaacaagaaataacactattgtgtggagaaattctatatgctaagcatgctgattacaaatatgctgcagaaataggaatacaatatattagcacagctctaggatcagagagagtacagcagattctaagaaactcaggcagtgaagtccaagtggttttaaccagaacgtactccttggggaaagttaaaaacaacaaaggagaagatttacagatgttagacatacacggagtagagaaaagctgggtggaagagatagacaaagaagcaagaaaaacaatggcaactttgcttaaagaatcatcaggcaatattccacaaaatcagaggccttcagcaccagacacacctataatcttattatgtgtaggtgccttaatatttaccaaactagcatcaactatagaagtgggattagagaccacagtcagaagagctaaccgtgtactaagtgatgcactcaaaagataccctaggatggacataccaaaaatcgctagatctttctatgatttatttgaacaaaaagtgtattacagaagtttgttcattgagtatggcaaagcattaggctcatcctctacaggcagcaaagcagaaagtttattcgttaatatattcatgcaagcttacggtgctggtcaaacaatgctgaggtggggagtcattgccaggtcatctaacaatataatgttaggacatgtatctgtccaagctgagttaaaacaagtcacagaagtctatgacctggtgcgagaaatgggccctgaatctgggctcctacatttaaggcaaagcccaaaagctggactgttatcactagccaattgtcccaactttgcaagtgttgttctcggcaatgcctcaggcttaggcataataggtatgtatcgcgggagagtgccaaacacagaactattttcagcagcagaaagctatgccaagagtttgaaagaaagcaataaaattaacttttcttcattaggactcacagatgaagaaaaagaggctgcagaacactttctaaatgtgagtgacgacagtcaaaatgattatgagtaattaaaaaagtgggacaagtcaaaatgtcattccctgaaggaaaagatattcttttcatgggtaatgaagcggcaaaattggcagaagctttccaaaaatcattaagaaaacctagtcataaaagatctcaatctattataggagaaaaagtgaacactgtatctgaaacattggaattacctactatcagtagacctaccaaaccgaccatattgtcagagccgaagttagcatggacagacaaaggtggggcaatcaaaactgaagcaaagcaaacaatcaaagttatggatcctattgaagaagaagagtttactgagaaaagggtgctgccctccagtgatgggaaaactcctgcagaaaagaagttgaaaccatcaaccaatactaaaaagaaggtctcatttacaccaaatgaaccaggaaaatacacaaagttggagaaagatgctctagacttgctttcagacaatgaagaagaagatgcagaatcctcaatcttaaccttcgaagaaagagatacttcatcattaagcattgaagccagactagaatcgattgaggagaaattaagcatgatattagggctattaagaacactcaacattgctacagcaggacccacagcagcaagagatgggatcagagatgcaatgattggcataagggaggaactaatagcagacataataaaagaagccaagggaaaagcagcagaaatgatggaagaagaaatgaaccagcggacaaaaataggaaacggtagtgtaaaattaactgaaaaggcaaaggagctcaacaaaattgttgaagacgagagcacaagtggtgaatccgaagaagaagaagaactaaaagacacacaggaaaataatcaagaagatgacatttaccagttaattatgtagtttaataaaaataaaaaatgggacaagtgaaaatggagtcctatctggtagacacctatcaaggcatcccttacacagcagctgttcaagttgatctagtagaaaaggacctgttacctgcaagcctaacaatatggttccccctgtttcaggccaatacaccaccagcagttctgcttgatcagctaaagactctgactataactactctgtatgctgcatcacaaagtggtccaatactaaaagtgaatgcatcggcccagggtgcagcaatgtctgtacttcccaaaaagtttgaagtcaatgcgactgtagcacttgacgaatatagcaaattagaatttgacaaacttacagtctgtgaagtaaaaacagtttacttaacaaccatgaaaccatatgggatggtatcaaagtttgtgagctcggccaaaccagttggcaaaaaaacacatgatctaatcgcattatgcgattttatggatctagaaaagaacacaccagttacaataccagcatttatcaaatcagtttctatcaaggagagtgaatcagccactgttgaagctgcaataagcagtgaagcagaccaagctctaacacaagccaaaattgcaccttatgcgggactgatcatgattatgaccatgaacaatcccaaaggcatattcaagaagcttggagctgggacccaagttatagtagaactaggagcatatgtccaggctgaaagcataagtaaaatatgcaagacttggagccatcaaggaacaagatatgtgctgaagtccagataacagccaagcaacctgaccaagaactaccaactctattctatagactaaaaagtcgccattttagttatataaaaatcaagttagaataagaattaaatcaatcaagaacgggacaaataaaaatgtcttggaaagtggtgatcattttttcattgctaataacacctcaacacggtcttaaagagagctacctagaagaatcatgtagcactataactgagggatatcttagtgttctgaggacaggttggtataccaacgtttttacattagaggtgggtgatgtagaaaaccttacatgttctgatggacctagcctaataaaaacagaattagatctgaccaaaagtgcactaagagagctcaaaacagtctctgctgaccaattggcaagagaggaacaaattgagaatcccagacaatctaggtttgttctaggagcaatagcactcggtgttgcaacagcagctgcagtcacagcaggtgttgcaattgccaaaaccatccggcttgagagtgaagtcacagcaattaagaatgccctcaaaacgaccaatgaagcagtatctacattggggaatggagttcgagtgttggcaactgcagtgagagagctgaaagactttgtgagcaagaatttaactcgtgcaatcaacaaaaacaagtgcgacattgatgacctaaaaatggccgttagcttcagtcaattcaacagaaggtttctaaatgttgtgcggcaattttcagacaatgctggaataacaccagcaatatctttggacttaatgacagatgctgaactagccagggccgtttctaacatgccgacatctgcaggacaaataaaattgatgttggagaaccgtgcgatggtgcgaagaaaggggttcggaatcctgataggggtctacgggagctccgtaatttacatggtgcagctgccaatctttggcgttatagacacgccttgctggatagtaaaagcagccccttcttgttccggaaaaaagggaaactatgcttgcctcttaagagaagaccaagggtggtattgtcagaatgcagggtcaactgtttactacccaaatgagaaagactgtgaaacaagaggagaccatgtcttttgcgacacagcagcgggaattaatgttgctgagcaatcaaaggagtgcaacatcaacatatccactacaaattacccatgcaaagtcagcacaggaagacatcctatcagtatggttgcactgtctcctcttggggctctggttgcttgctacaaaggagtaagctgttccattggcagcaacagagtagggatcatcaagcagctgaacaagggttgctcctatataaccaaccaagatgcagacacagtgacaatagacaacactgtatatcagctaagcaaagttgagggtgaacagcatgttataaaaggcagaccagtgtcaagcagctttgatccaatcaagtttcctgaagatcaattcaatgttgcacttgaccaagtttttgagaacattgaaaacagccaggccttggtagatcaatcaaacagaatcctaagcagtgcagagaaagggaatactggcttcatcattgtaataattctaattgctgtccttggctctagcatgatcctagtgagcatcttcattataatcaagaaaacaaagaaaccaacgggagcacctccagagctgagtggtgtcacaaacaatggcttcataccacacagttagttaattaaaaataaaataaaatttgggacaaatcataatgtctcgcaaggctccatgcaaatatgaagtgcggggcaaatgcaacagaggaagtgagtgtaagtttaaccacaattactggagttggccagatagatacttattaataagatcaaactatctattaaatcagcttttaaggaacactgatagagctgatggcctatcaataatatcaggcgcaggcagagaagacagaacgcaagattttgttctaggttccaccaatgtggttcaaggttatattgatgataaccaaagcataacaaaagctgcagcctgctacagtctacacaacataatcaagcaactacaagaagttgaagttaggcaggctagagatagcaaactatctgacagcaagcatgtggcactccataacttaatcttatcttacatggagatgagcaaaactcccgcatctttaatcaacaatctcaaaagactgccgagagaaaaactgaaaaaattagcaaagctgataattgacttatcagcaggcgctgacaatgactcttcatatgccctgcaagacagtgaaagcattaatcaagtgcagtgagcatggtcctgttttcattactatagaggttgatgaaatgatatggactcaaaaagaattaaaagaagctttgtccgatgggatagtgaagtctcacaccaacatttacaattgttatttagaaaacatagaaattatatatgtcaaggcttacttaagttagtaaaaacacatcagagtgggataagtgacaatgataacattagatgtcattaaaagtgatgggtcttcaaaaacatgtactcacctcaaaaaaataatcaaagaccattctggtaaagtgcttattgcacttaagttaatattagctttactaacatttttcacaataacaatcactataaattacataaaagtagaaaacaatctacaaatatgccagtcaaaaactgaatcagacaaagaagactcaccatcaaataccacatccgtcacaaccaagaccactctagaccatgatataacacagtattttaaaagattaattcaaaggtatacagattctgtaataaacaaggacacatgctggaaaataagcagaaatcaatgcacaaatataacaacatataaatttttatgctttaaacctgaggactcaaaaatcaacagttgtgatagactgacagatctatgcagaaacaaatcaaaatcagcagctgaagcatatcatacagtagaatgccattgcatatacacaattgagtggaagtgctatcaccactcaatagattaaacccaatcttgaatgttaaaactagactaggatccgtctaagactatcagttcaatagtttagttattttaaaatatttgagaataggtaagtttctatggcacttcatagcaataggtaataattaacagcttaattataattaaaacattatttaaaatcgtaactatttaatttacaaagtaaaaacaaaaatatgggacaagtagttatggaggtgaaagtagagaacattcgagcaatagacatgctcaaagcaagagtgaaaaatcgtgtggcacgtagcaaatgctttaaaaatgcttctttaatcctcataggaataactacactgagtatagctctcaatatctatctgatcataaactacacaatacaaaaaacctcatctgaatcagaacaccacaccagctcaccacccacagaatccaacaaggaagcttcaacaatctccacagacaacccagacatcaatccaaactcacagcatccaactcaacagtccacagaaaaccccacactcaaccccgcagcatcagtgagcccatcagaaacagaaccagcatcaacaccagacacaacaaaccgcctgtcctccgtagacaggtccacagcacaaccaagtgaaagcagaacaaagacaaaaccgacagtccacacaagaaacaacccaagcacagcttccagtacacaatccccaccacgggcaacaacgaaggcaatccgcagagccaccactttccgcatgagcagcacaggaaaaagaccaaccacaacatcagtccagtccgacagcagcaccacaacccaaaatcatgaagaaacaggttcagcgaacccacaggcatctgtaagcacaatgcaaaactagcacaccaacaatataaaaccaaattagttaacaaaaaatacgagatagctctaaagtaaaacatgtaggtaccaacaatcaagaaaccaaaagacaactcacaatctccctaaaacagcaacgacaccatgtcagctttgctcaaatctctctgggagaaacttttgcccacatactaacaacatcacaaccatctcaagaaaagaaactgggcaaaacagcatccaagagacaaatagcaatggatcctcttaatgaatccactgttaatgtctatctccctgattcgtaccttaaaggagtaatttcttttagtgaaactaatgcaattggttcatgtctcttaaaaagaccctacttaaaaaatgacaacactgcaaaagttgccatagagaatcctgttattgagcatgtgagactcaaaaatgcagtcaattctaaaatgaaaatatcagattacaaggtagtagagccagtaaacatgcaacatgaaataatgaagaatgtacacagttgtgagctcacactattgaaacagtttttaacaaggagtaaaaacattagcactctcaaattgaatatgatatgtgattggctgcaattaaagtctacatcagatgatacctcaatcctaagtttcatagatgtagaatttatacctagttgggtaagcaactggtttagtaattggtacaatctcaataagttaattttggaattcagaagagaggaagtaataagaaccggttcaatcttatgcaggtcattgggtaaattagtttttattgtatcatcatatggatgtatcgtcaagagcaacaaaagcaaaagagtgagcttcttcacatacaatcaactgttaacatggaaagatgtgatgttaagtagatttaatgcgaatttttgtatatgggtaagcaatagtctgaatgaaaatcaggaagggctagggttgagaagtaatctgcaaggtatgttaactaataaactatatgaaactgtagattatatgctaagcttatgttgcaatgaaggtttctcacttgtgaaagagttcgaaggttttattatgagtgagatccttaggattactgaacatgctcaattcagtactagatttagaaatactttattgaatggattaacagatcaattaacaaaattaaaaaataaaaacagactcagagttcatagtaccgtattagaaaataatgattatccaatgtatgaagttgtacttaaattattaggagatactttgagatgtatcaaattattaatcaataaaaacttagagaatgctgcagaattatactatatattcagaatttttggtcatccaatggtagatgaaagagatgcaatggatgctgtcaaattaaacaatgaaatcacaaaaatcctaaggttggagagcttgacagaactaagaggggcattcatattaaggattatcaaaggatttgtggacaacaacaaaaggtggcccaaaattaaaaacttaaaagtgcttagcaaaagatggactatgtacttcaaagctaaaaattaccccagtcaactcgaattaagtgaacaagactttctagagcttgctgcaatacaatttgaacaagagttttctgttcctgaaaaaaccaatcttgagatggtattaaatgacaaagccatatcacctcctaaaagattaatatggtctgtgtatccaaagaattacttacctgagacgataaaaaatcgatatttagaagaaactttcaatgcgagtgatagtctcaaaacaagaagagtactagagtactatttaaaagataataaatttgatcaaaaggaacttaaaagttatgtagttagacaagaatatttaaacgataaggagcacattgtctcattaactggaaaagaaagagaattaagtgtaggtagaatgtttgctatgcaaccaggaaaacagcgacaaatacaaatattggcagaaaaattgttagctgataacattgtacctttcttcccggaaaccttaacaaagtatggtgatctagatcttcagagaataatggaaatcaaatcagaactttcttctatcaaaaccagaagaaatgatagttataataattacattgcaagagcatccatagtaacagatttgagcaagttcaaccaagcctttagatatgaaactacagcgatctgtgcggatgtagcagacgaattacatggaacacaaagcttattctgttggttacatcttatcgttcctatgactacaatgatatgtgcctatagacatgcaccaccagaaacaaaaggtgaatatgatatagataagatagaagagcaaagtggtctatatagatatcacatgggcggtattgaaggatggtgtcaaaaactctggacaatggaagctatatctttattggatgttgtatctgtaaagacacggtgtcaaatgacatctttattaaacggtgacaaccaatcaatagatgtaagtaaaccagtcaagttatctgaaggtttagatgaagtgaaggcagattatcgcttagcagtaaaaatgctaaaagaaataagagatgcatacagaaatataggccataaacttaaagaaggggaaacatatatatcaagggatcttcagtttataagcaaggtgattcaatctgaaggagtgatgcatcctacccctataaaaaaggtcttgagagtaggaccatggataaacacaatattagatgacattaaaactagtgctgagtcaatagggagtctatgtcaagaattagaatttaggggagaaagcataatagttagtctgatattaagaaacttctggctgtataacttatacatgcatgaatcaaagcaacatcctttggcagggaaacagttattcaaacaactaaataaaacattaacatcagtgcagagattttttgaaattaaaagggaaaatgaggtagtagatctatggatgaacataccaatgcaatttggaggaggagatccagtagtcttctatagatctttctatagaaggacccctgattttttaactgaggcaatcagccatgtagatattctgttaaaaatatcagctaacataaaaaatgaaacgaaagtaagtttcttcaaagccttactatcaatagaaaaaaatgaacgtgctacactgacaacactaatgagagatcctcaagctgttggatcagaacgacaagcaaaagtaacaagtgacatcaatagaacagcagttaccagtatcttaagtctttccccaaatcaacttttcagtgatagtgctatacactacagcagaaatgaagaagaagtgggaatcattgcagaaaacataacacctgtttatcctcatgggctgagagtattatatgaatcattgccctttcacaaagctgaaaaagttgtaaacatgatatcagggacaaaatctataaccaacttattacagagaacatccgctattaatggtgaagatattgacagggctgtatctatgatgttggagaatctaggattattatctagaatattgtcagtagttgttgatagtatagaaattccaatcaaatctaatggtaggctgatatgttgtcaaatctctaggactttaagagagacatcatggaataatatggaaatagttggagtaacatctcctagcatcactacatgtatggatgtcatatatgcaactagttctcatttgaaagggataattatagaaaagttcagcactgacagaactacaaggggtcaaagaggtccaaaaagcccttgggtagggtcgagtactcaagagaaaaaattagtacctgtttataacagacaaattctctcaaaacaacaaagagaacagctagaagcaattggaaaaatgagatgggtgtataaagggacaccaggcttgcgacgattactcaacaagatctgtcttgggagtttaggcattagctacaaatgtgtaaaacctttattacctaggtttatgagtgtaaatttcttacatagattatctgtcagtagtagacctatggaattcccagcatcagttccagcttatagaacaacaaattaccatttcgacactagtcctattaatcaagcactaagtgagagatttgggaatgaagatattaacttggtcttccaaaatgcaatcagctgtggaattagcataatgagtgtagtagaacaattaacaggtagaagcccaaaacagttagttttaataccccaattagaagaaatagacattatgccaccaccagtgtttcaagggaaattcaattataaattagtagataagataacttctgatcaacatatctttagtccggacaaaatagatatgttaacactagggaaaatgctcatgcccactataaaaggtcagaaaacagatcagttcttaaataagagagaaaattatttccatggaaacaatcttattgagtctttatcagcagcattagcatgtcattggtgtgggatattaacagaacaatgcatagaaaataatattttcaagaaagactggggtgacgggtttatatcagatcatgcttttatggacttcaaaatattcctatgtgtctttaaaactaaacttttatgtagttggggatctcaagggaaaaacattaaagatgaagatatagtagatgaatcaatagataaattgttaaggattgacaatactttttggagaatgttcagcaaagttatgtttgaaccaaaggttaagaaaaggataatgttatatgatgtaaaattcctatcactagtaggctacatagggtttaagaactggtttatagagcagttgagatcagctgaattgcatgaaataccttggattgtcaatgccgaaggtgatttggttgagatcaagtcaattaaaatctatttgcaactgatagaacaaagcttatttttaagaataactgttttgaactatacagatatggcacatgctctcacacgattaatcagaaagaagttaatgtgtgataatgcactgttaaccccaatttcatccccaatggttaacttaactcaagttattgatcccacaacacaattagattacttccccaagataacattcgaaaggctaaaaaattatgacacaagttcaaattatgctaaagggaagctaacaagaaattacatgatactattgccatggcagcatgttaatagatataactttgtctttagttctactggatgtaaagttagtctgaaaacatgtattggaaaacttatgaaagacctaaatcctaaagttttgtactttattggagaaggagcaggaaattggatggccagaacagcatgtgaatatcctgatattaaatttgtatatagaagtctgaaagatgaccttgatcatcattatcctctggaataccagagagtgataggtgaattaagcagaatcatagatagtggtgaaggactttcaatggaaacaacagacgcaactcaaaaaactcattgggatttgatacacagggtaagcaaagatgctttattaataactttatgtgatgcagaatttaaggacagagatgatttttttaagatggtaattctatggagaaaacatgtattatcatgcagaatttgcactacttatgggacggacctctatttattcgcaaagtatcatgctaaagactgcaatgtaaaattacctttttttgtgagatcagttgctactttcattatgcagggtagtaagctgtcaggttcagaatgctacatactcttaacactaggccaccacaacagtttaccttgccatggagaaatacaaaattctaagatgaaaatagcagtgtgtaatgatttttatgctgcaaaaaaactcgacaataaatcaattgaagctaattgtaaatcacttttgtcagggctaagaatacctataaataagaaggaactagatagacagagaagattattaacactacaaagcaatcattcttctgtagcaacagttggcggtagcaagatcatagagtctaagtggttaacaaacaaagcaagtacaataattgattggttagaacatattttaaattctccaaagggcgaattaaattatgatttttttgaagcattggagaacacttaccctaatatgattaaactaatagataacttagggaatgcagagattaaaaaactgatcaaagtaacaggatacatgcttgtaagtaaaaaatgaaaaatgatgaagatgacaaaatagatgacaacttcatactattctaaattaattatttgattatgcaattatatgatagttaattaaaattaaaaattaaaaatcaaaagttaaaatttaaaacctatcattaagtttattaaaaataagaaattataattgaatgtatacggtttttttgccgt

SEQ ID NO: 174 - DNA, 13280 bp, Viruses
  CAN9875 genomic DNA
acgcgaaaaaaacgcgtataaattaaattccaaacaaaacgggacaaataaaaatgtctcttcaagggattcacctaagtgatctgtcatataaacatgctatattaaaagagtctcaatacacaataaaaagagatgtaggcaccacaactgcagtgacaccttcatcattgcagcaagagataacacttttgtgtggagagattctttacactaaacatactgattacaaatatgctgcagagatagggatacaatatatttgcacggctctaggatcagaaagagtacaacagattttaagaaattcaggcagtgaagttcaggtggttctaaccaagacatactctttagggaaaggtaaaaatagtaaaggggaagagttgcaaatgttagatatacatggagtggaaaagagttgggtagaagaaatagacaaagaggcaagaaaaacaatggtgactttgctaaaggaatcatcaggtaacatcccacaaaaccagaggccttcagcaccagacacaccaataattttattatgtgtaggtgctttaatattcactaaactagcatcaacaatagaagttggactagagactacagttagaagagctaacagagtgctaagtgatgcgctcaaaagataccctagggtagatataccgaagattgctagatctttctatgaactatttgagcagaaagtgtattacaggagtctattcattgagtatgggaaagctttaggctcatcttcaacaggaagcaaagcagaaagtttgtttgtaaatatatttatgcaagcttatggagccggtcaaacaatgctaaggtggggtgtcattgccagatcatctaacaacataatgctaggacatgtgtctgtgcaagctgaattgaagcaagttacagaggtttatgatttggtgagagaaatgggtcctgaatctgggcttttacatctaagacaaagtccaaaggcaggactgttatcgttggccaattgccccgattttgctagtgttgttcttggtaatgcttcaggtctaggtataatcggaatgtacagaggaagagtgccaaacacagagctattttctgcagcagaaagttatgccagaagcttaaaagaaagcaacaaaatcaacttctcctcattagggctcacagacgaagaaaaagaagctgcagaacacttcttaaacatgagtgatgacaatcaagatgattatgagtaattaaaaaactgggacaagtcaaaatgtcattccctgaaggaaaagatatcctgttcatgggtaatgaagcagcaaaaatagcagaagctttccagaaatcactaaaaagatcaggtcacaaaagaacccagtctattgtaggggaaaaagtaaacactatatcagaaactctagagctacctaccatcagcaaacctgcacgatcatctacactgctagagccaaaattggcatgggcagacagcagcagagccaccaaaaccacagaaaaacaaacaaccaaaacaacagatcctgttgaagaagaggaactcaatgaaaagaagatatcaccttccagtgatgggaagactcccgcagagaaaaaatcaaaatctccaaccaatgtaaaaaagaaagtttccttcacatcaaatgaaccagggaaatataccaaactagaaaaagatgccctagatttgctctcagacaatgaggaagaagacgcagagtcctcaatcttaacctttgaagagagagacacatcatcactaagcattgaggctagactagaatcaatagaagagaagctaagcatgatattaggactgcttcgtacacttaacattgcaacagcaggaccaacggctgcaagagatggaatcagagatgcaatgattggtataagagaagaactaatagcagaaataataaaagaagcaaagggaaaagcagctgaaatgatggaagaggaaatgaatcaaaggtcaaaaataggtaatggcagtgtaaaactaaccgagaaggcaaaagaacttaataaaattgttgaagacgagagcacaagcggtgaatcagaagaagaagaagaaccaaaagaaactcaggataacaatcaaggagaagatatttaccagttaatcatgtagtttaataaaaataaacaatgggacaagtcaagatggagtcctatctagtggacacttatcaaggcattccctacacagctgctgttcaagttgatctggtagaaaaagacttactaccagcaagtttgacaatatggtttcctctattccaagccaacacaccaccagcggttttgctcgatcagctaaagaccttgacaataacaactctgtatgctgcatcacagaatggtccaatactcaaggtaaatgcatcagctcagggtgctgctatgtctgtacttcccaaaaaattcgaagtaaatgcaactgtggcacttgatgaatacagcaaacttgactttgacaagttaacggtttgcgatgttaaaacagtttatttgacaaccatgaaaccatatgggatggtgtcaaaatttgtgagttcagccaaatcagttggcaacaagacacatgatctaattgcactgtgtgacttcatggacctagagaaaaatatacctgtgacaataccagcattcataaagtcagtttcaatcaaagagagtgagtcagccactgttgaagctgcaataagcagtgaggccgaccaagcattaacacaagccaaaattgcaccctatgcaggactaatcatgatcatgaccatgaacaatccaaaaggtatattcaagaaactaggagctggaacacaagtgatagtagagctaggggcatatgttcaagccgagagcatcagcaggatctgcaagagctggagtcaccaaggaacaagatatgtactaaaatccagataaaaataactgtcctaatcaataattgcttatataatcttaaagatcaatgagcttattattatagttatataaaaaaatttagaactaggaaggtattaatagaaagcgggacaagtaaaaatgtcttggaaagtgatgattatcatttcgttactcataacacctcagcacggactaaaggaaagttatttagaagaatcatgtagtactataactgaaggatatctcagtgttttaagaacaggttggtacaccaatgtctttacattagaagttggtgatgttgaaaatcttacatgtactgatggacctagcttaatcaaaacagaacttgacctaaccaaaagtgctctaagagaactcaaaacagtttctgctgatcagttagcgagagaagaacaaattgaaaatcccagacaatcaaggtttgtcctaggtgcaatagctcttggtgttgccacagcagcagcagtcacagcaggcattgcgatagccaaaaccataaggcttgagagtgaagtgaatgcaatcaaaggtgctctcaaaacaaccaatgaggcagtatccacactaggaaatggagtgcgagtcctagccaccgcagtaagagagctgaaagaatttgtgagcaaaaacctgactagtgcaattaacaagaacaaatgtgacattgctgatctgaagatggctgtcagcttcagtcaattcaacagaagattcctaaatgttgtgcggcagttttcagacaatgcagggataacaccagcaatatcattggacctaatgactgatgctgagctggccagagctgtatcatacatgccaacatctgcaggacagataaaactaatgttagagaaccgtgcaatggtgaggagaaaaggatttggaatcttgataggggtctacggaagctccgtgatttacatggtccagctgccgatctttggtgtcatagatacaccttgttggataatcaaggcagctccctcttgttcagaaaaagatggaaactatgcttgcctcctaagagaggatcaagggtggtattgtaaaaatgcaggatccactgtttactacccaaataaaaaagactgcgaaacaagaggtgatcatgttttttgtgacacagctgcagggatcaatgttgctgagcaatcaagagaatgcaacatcaacatatctacaaccaactacccatgcaaagtcagcacaggaagacaccctatcagcatggttgcactatcacctctcggtgctttggtggcttgctacaaaggggttagctgttcaattggcagtaatcgggttggaataatcaaacaactacctaaaggctgctcatacataactaaccaggacgcagacactgtaacaattgacaacactgtgtatcaactaagcaaagttgagggtgaacagcatgtaataaaagggagaccagtttcaagcagtttcgatccaatcaagtttcctgaggatcagttcaatgttgcgcttgatcaagtctttgaaagcattgaaaacagtcaagcactagtggaccagtcaaacaaaattctgaacagtgcagaaaaaggaaacactggcttcattattgtaataattttgattgctgttcttgggttaaccatgatttcagtgagcatcatcatcataatcaaaaaaacaaggaaacccacaggggcacctccagagctgaatggtgttaccaacggcggttttataccgcatagttagttaattaaaaaatgggacaaatcatcatgtctcgtaaagctccatgcaaatatgaagtacggggcaagtgcaacaggggaagtgagtgcaaattcaaccacaattactggagttggcctgataggtatttattgttaagatcaaattatctcttgaatcagcttttaagaaacactgataaggctgatggtttgtcaataatatcaggagcaggtagagaagacaggactcaagactttgttcttggttctactaatgtggttcaagggtacattgatgacaatcaaggaataacaaaggctgcagcttgctatagtctacataacataataaaacagctacaagaaatagaagtaagacaggccagagataataagctttctgacagcaaacatgtggcacttcacaacttgatattatcctatatggagatgagcaaaactcctgcatccctgattaataacctaaagaaactaccaagagaaaaactgaaaaaattagcgaaattaataattgatttatcagcaggaactgataatgactcttcatatgccttgcaagacagtgaaagcactaatcaagtgcagtaagcatggtcccaaattcatcaccatagaggcagatgatatgatatggacacacaaagaattaaaagagacactgtctgatgggatagcaaaatcacacaccaatatttacagttgttatttagaaaatatagaaataatatatgttaaagcttacttaagttagtaaaaaataaatagaatgggataaatgacaatgaaaacattagatgtcataaaaagtgacggatcctcagaaacatgtaatcaactcaaaaaaataataaaaaaacactcaggtaaattgcttattgcatcaaaaccgacattggccttattgacgtccttcacagtaacaattactgtcaactatacaaaagtagaaaataatttgcaggcatgtcaattaaaaaatgaatcagacaaaaaggacacaaagctaaataccacatcaacaacaatcagacccattcctgatctaaatgcagtacagtacctgaaaaggctgattcagaaacacaccaactctgtcacaaaagacagagatacctgttggagaatacacacgaatcaatgcacaaatataaaaatatataagttcttatgttttgggtctatgaattcaacaaatacagactgtgaagaaccaacagttctatgcgacaaaaagtcaaaaaccatgacagaaaaacataggaaagcagagtgtcaccgtccacatacaaccgagtggtggtgccattatctttaagagaaaactcagttttcaacattaaaatcagaacaaatcatatctagatctattaatataatagtctagttatttaaaaactctaaatattgtctagacttcacaacaccctgcggtcatatgcaataatcaatggtcaaaccactgttgcaaacccacctataatacaatcactgagtaatacaaaacaagaaaatgggacaagtggccatggaagcaagagtggagaacattcgggcaatagacatgttcaaagcaaagatgaaaaaccgtataagaagtagcaagtgccatagaaatgctacactgatccttattggatcaacagcaccaagtatggcactcaacacccttttaatcattgatcatgcaacatcaaaaaacatgaccaaagtggaacactgtgtcaacatgccgccggtagaaccaagcaagaagaccccaatgacctctgcagcagacccaaacaccaaacccaatccacagcaggcaacacagctgaccacagaggattcaacatctctagcagcaaccctagaggaccatctacacacagggacaactccaacaccagatgcaacagtctcccagcaaaccacagacgagcacacaacactgctgagatcaaccaacagacagaccacccaaacaaccgcagagaaaaagccaaccagagcaacaaccaaaaaagaaaccacaactcgaaccacaagcacagctgcaacccaaacactcaacaccaccaaccaaactagcaatggaagagaggcaaccacaacatctgccagatccagaaacaatgccacaactcaaagcagcgatcaaacaacccaggcagcagacccaagctcccaatcacaacatacacagaaaagcacaacaacaacacacaacacagacacatcttctccaagtagttaacaaaaaaactataaaataaccatgaaaaccaaaaaactagaaaagttaatttgaactcagaaaagaacacaaacactatatgaattgtttgagcgtatatactaatgaaatagcatctgtttgtgcatcaataataccatcattatttaagaaataagaagaagctaaaattcaagggacaaataacaatggatccgttttgtgaatccactgtcaatgtctatcttcctgattcatatctcaaaggagtaatatctttcagtgaaaccaatgcaattggctcatgccttttgaaaagaccctatcttaaaaaagataacactgctaaagttgctgtagaaaaccctgttgttgaacatgtcagacttagaaatgcagtcatgaccaaaatgaagatatcagattataaagtggttgaaccaattaatatgcagcatgaaataatgaaaaatatacacagttgtgagctcacattattaaaacaattcttaacaagaagtaaaaacattagctccctaaaattaagtatgatatgtgattggttacagttaaaatccacctcagataacacatcaattcttaattttatagatgtggagtttatacccgtttgggtgagcaattggtttagtaactggtataatctcaataaattaatcttagagtttagaagagaggaagtaataagaactggttcaattttatgcagatcactaggcaagttagttttcattgtatcatcttatgggtgtgtagtaaaaagcaacaaaagtaaaagagtaagttttttcacatataaccaactgttaacatggaaagatgtgatgttaagtaggttcaatgcaaacttttgtatatgggtaagtaacaacctgaacaaaaatcaagaaggactaggatttagaagtaatctacaaggtatgttaactaataaattatatgaaactgttgattatatgttaagtctatgtagcaatgaagggttctcactagtgaaagagttcgaaggctttattatgagtgaaattcttaaaattactgagcatgctcaattcagtactaggtttaggaatactttattaaatgggttgactgaacaattatcaatgttgaaagctaaaaacagatctagagttcttggcactatattagaaaacaatgattaccccatgtatgaagtagtacttaaattattaggggacactttgaaaagtataaaattattaattaacaagaatttagaaaatgctgcagaattatattatatattcagaatttttggacaccctatggtagatgagagggaagcaatggatgctgttaaattaaataatgagattacaaaaattcttaaactggagagcttaacagaactaagaggagcatttatactaagaattataaaagggtttgtagataataataaaagatggcctaaaattaagaatttaaaagtgctcagtaaaagatgggttatgtatttcaaagctaaaagttaccctagccaacttgagctaagtgtacaagattttttagaacttgctgcagtacaattcgaacaggaattttctgtccctgaaaaaaccaatcttgagatggtattaaatgataaagcaatatctccaccaaaaaagttaatatggtcggtatatccaaaaaattatctacctgaaattataaaaaatcaatatttagaagaggtcttcaatgcaagtgacagtcaaagaacgaggagagtcttagaattttacttaaaagattgcaaatttgatcaaaaagacctcaaacgttatgtaactaaacaagagtatctaaatgacaaagaccacattgtctcattaactgggaaagaaagagaattaagtgtaggcaggatgtttgcaatgcaacctggcaaacaaagacaaatacagatactagccgagaaacttttagctgataatattgtaccctttttcccagaaactttaacaaagtatggtgacttggatctccaaagaattatggaaatgaaatcagaactttcttccattaaaactaggaagaatgatagttacaacaattatattgcaagagcctccatagtaacagacctaagtaaattcaatcaagcctttagatatgaaaccacagctatctgcgcagacgtagcagatgagttacatggcacgcaaagcttattttgttggttacatcttattgttcccatgaccacaatgatatgtgcatacagacatgcaccaccagaaacaaagggggagtatgatatagacaaaatagaagagcaaagtgggctatacagataccatatgggagggattgaagggtggtgtcagaagttatggacaatggaggcgatatccttgttagatgtagtatctgttaagactcgttgtcagatgacctctctattaaacggagacaatcaatcaatagatgtcagtaaaccagtaaaattgtctgaaggtatagatgaagtaaaagcagattatagcttagcaattaaaatgcttaaagagataagagatgcctataaaaacattggccataaactcaaagaaggtgaaacatatatatcaagagatcttcaatttataagtaaggtgattcaatctgagggggtcatgcatcctacccccataaaaaagatattaagggtaggtccctggataaatacaatactagatgacattaaaactagtgcagaatcaatagggagtctgtgtcaagaactagagttcagaggagaaagtatactagttagcttgatattaaggaatttctggctgtataacttatacatgcatgagtcaaaacagcatccgttagctggaaaacaactgtttaaacaattgaacaaaacactaacatctgtgcaaagattttttgagctgaagaaagaaaatgatgtggttgacctatggatgaatataccaatgcagtttggagggggagacccagtagttttttacagatctttttacagaaggactcctgatttcttgactgaagcaatcagccatgtggatttactgttaaaagtttcaaacaatattaaaaatgagactaagatacgattctttaaagccttattatctatagaaaagaatgaacgtgctacattaacaacactaatgagagacccccaggcggtaggatcggaaagacaagctaaggtaacaagtgatataaatagaacagcagttactagcatactgagtctatctccgaatcagctattttgtgatagtgctatacactatagcagaaatgaagaagaagtagggatcattgcagacaacataacacctgtttatcctcacggattgagagtgctctatgaatcactaccttttcataaggctgaaaaggttgtcaatatgatatcaggtacaaagtctataactaacctattgcagagaacatctgctatcaatggtgaagatattgatagagcagtgtctatgatgttagagaacttagggttgttatctaggatattgtcagtaataattaatagtatagaaataccaattaagtccaatggcagattgatatgctgtcaaatttctaagactttgagagaaaaatcatggaacaatatggaaatagtaggagtgacatctccaagtattgtaacatgtatggatgttgtgtatgcgactagttctcatttaaaaggaataattattgaaaaattcagtactgacaagaccacaagaggtcagaggggaccaaaaagcccttgggtaggatcaagcactcaagagaaaaaattagttcctgtttataacagacaaattctttcaaaacaacaaaaagagcaactggaagcaataggaaaaatgaggtgggtgtataaaggaactccagggctaagaagattgctcaataagatttgcataggaagtttaggtattagctataaatgtgtaaaacctctattaccaagatttatgagtgtaaacttcttacataggttatctgttagtagcagacccatggaattcccagcttctgttccagcttataggacaacaaattaccactttgacactagtccaatcaaccaagcattaagtgagaggttcgggaacgaagacattaatctagtgttccaaaatgcaatcagctgcggaattagtataatgagtgttgtagaacagttaactggtagaagcccaaaacaattagtcttaatcccccaattagaagagatagatattatgccccctcctgtatttcaaggaaaattcaattataaactagttgataaaataacctccgatcaacacatcttcagtcctgacaaaatagacatattaacactagggaagatgcttatgcctactataaaaggtcaaaaaactgatcagttcttaaataagagagaaaactatttccatggaaataatttaattgaatctttatctgcagcacttgcatgccattggtgtggaatattaacagaacagtgtgtagaaaacaatatctttaggaaagactggggtgatgggttcatatcagatcatgccttcatggatttcaagatatttctatgtgtatttaaaaccaaacttttatgtagttggggatcccaagggaaaaatgtaaaagatgaagatataatagatgaatccattgacaaattattaagaattgacaacactttttggagaatgttcagcaaagtcatgtttgaatcaaaggtcaaaaaaagaataatgttatatgatgtaaaattcctatcattagtaggttatataggatttaaaaactggtttatagagcagttaagagtagtagaattgcatgaagtaccctggattgtcaatgctgaaggggagctagttgaaattaaaccaatcaaaatttatttgcagttaatagaacaaagtctatctttaagaataactgttttgaattatacagacatggcacatgctcttacacgattaattaggaagaaattgatgtgtgataatgcactctttaatccaagttcatcaccaatgtttagtctaactcaagttattgatcctacaacacagctagactattttcctaaggtaatatttgaaaggttaaaaagttatgacaccagttcagactacaacaaagggaagttaacaagaaattacatgacattattaccatggcagcacgtaaacaggtataattttgtctttagttcaacaggatgtaaaatcagcttgaagacatgcatcgggaaattgataaaggacttaaaccctaaggttctttactttattggagaaggagcaggtaactggatggcaagaacagcatgtgagtatcctgacataaaatttgtatataggagtttaaaggatgatcttgatcaccattacccattagaatatcaaagggtaataggtgatttaaatagagtaatagatggtggtgaaggattatcaatggagaccacagatgcaactcaaaagactcattgggacttgatacacagaataagtaaagatgctttattgataacattgtgtgatgcagaattcaaaaacagagatgatttctttaaaatggtaattctttggagaaaacatgtattatcatgtagaatctgtacagcttatggaacagatctttacttatttgcaaagtatcatgcgacggactgcaatataaagttaccattttttgtaaggtctgtagctacttttattatgcaaggaagcaaattgtcaggatcagaatgttacatacttttaacattaggtcatcacaataatctgccatgtcacggagaaatacaaaattccaaaatgagaatagcagtgtgtaatgatttccatgcctcaaaaaaactagacaacaaatcaattgaagcaaactgcaaatctcttctatcaggattaagaataccaataaacaaaaaagagttaaatagacaaaagaaactgttaacactacaaagcaatcattcttccatagcaacagttggcggaagtaagattatagaatccaaatggttaaagaataaagcaagtacaataattgattggttagagcatatcttgaattctccaagaggtgaattaaactatgatttctttgaagcattagagaacacatatcccaatatgatcaagcttatagataacctgggaaatgcagagataaaaaaactaatcaaagttaccgggtatatgcttgtgagtgagaagtaataataataataataatcaaccataatctcacacaactgagaaaatgatcatctaacagtttaattgaccattagttaattaaaaattataaattagtaactaattgataaaaaataagaaattgaaattgaatgtatacggtttttttgccgt